(19) United States
(12) Patent Application Publication
(10) Pub. No.: US 2006/0147922 A1
(43) Pub. Date: Jul. 6, 2006

(54) Novel diagnostic and therapeutic methods and reagents therefor

(76) Inventors: Colin Watts, Avalon, NSW (AU); Darren Saunders, Alexandria,New South Wales (AU); Michelle Henderson, Drummoyne, New South Wales (AU); Jennifer Clancy, Stanmore, New South Wales (AU); Susan Henshall, Mosman, New South Wales (AU); Robert Sutherland, Lindfield, New South Wales (AU); Philippa O'Brien, Woollahra, New South Wales (AU)

Correspondence Address: COZEN O'CONNOR, P.C., 1900 MARKET STREET, PHILADELPHIA, PA, 19103-3508, US

(21) Appl. No.: 10/526,905

(22) PCT Filed: Sep. 5, 2003
(86) PCT No.: PCT/AU03/01164

§ 371 Date: Feb. 23, 2006

Related U.S. Application Data
(60) Provisional application No. 60/425,218, filed on Nov. 7, 2002.

(30) Foreign Application Priority Data
Sep. 5, 2002 (AU) 2002951346

(51) Int. Cl. C12Q 1/68; G06F 19/00; C12P 19/34

(52) U.S. Cl. 435/6; 435/91.2; 702/20

(57) ABSTRACT

This invention provides novel methods of detecting or treating aberrant cell cycle regulation associated with expression of a nuclear protein encoded by a gene that is linked to map position 8q22.3 of the human genome, and to novel reagents that are useful therefor. More particularly, the invention provides novel nucleic acid and proteinaceous probes, for detecting a gene that is linked to map position 8q22.3 of the human genome or the expression products thereof, wherein expression or elevated expression of said gene is associated with the appearance or occurrence of tumors associated with cancer, DNA damage and progesterone-receptor-mediated effects on cells. The invention also provides reagents and methods for detecting or modulating the expression products of the gene, such as, for example, in the diagnosis or treatment of cancer, cellular proliferation, DNA damage or progesterone receptor-mediated effects on cells.

### FIELD OF THE INVENTION

This invention relates to novel methods of detecting or treating aberrant cell cycle regulation associated with expression of a nuclear protein encoded by a gene that is linked to map position 8q22.3 of the human genome, and to novel reagents that are useful therefor. More particularly, the invention relates to novel nucleic acid and proteinaceous probes, including antibodies, for detecting a gene that is linked to map position 8q22.3 of the human genome or the expression products thereof, wherein expression or elevated expression of said gene is associated with the appearance or occurrence of tumors associated with cancer, DNA damage and progesterone-receptor-mediated effects on cells. The invention also relates to reagents and methods for targeting the expression products (e.g. mRNA, protein, or protein-protein complexes) of the gene, such as, for example, in the prophylactic or therapeutic treatment of cancer or for reducing or preventing cell proliferation (e.g. in tumors), or for modifying progesterone receptor-mediated effects. In another embodiment, the invention relates to reagents and methods for detecting or modulating the expression products (e.g. mRNA, protein, or protein-protein complexes) of the gene, such as, for example, in the diagnosis or treatment of cancer, DNA damage or progesterone receptor-mediated effects on cells. In yet another embodiment, this invention relates to a variant of a gene that is linked to map position 8q22.3 of the human genome and to novel reagents and methods for detecting said variant.

### BACKGROUND OF THE INVENTION

1.General

This specification contains nucleotide and amino acid sequence information prepared using Patentin Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

2. Description of the Related Art

It is widely recognized that simple and rapid tests for hyperproliferative disorders have considerable clinical potential. Not only can such tests be used for the early diagnosis, but they also allow the detection of disease recurrence following treatment. However, the diagnosis of many hyperproliferative disorders, such as, for example, carcinoma of the ovary, is generally only possible when the disease has progressed to a late stage of development. Whilst previously identified markers for many carcinomas, e.g. carcinomas of the lung, prostate, breast, colon, pancreas, and ovary, have facilitated efforts to diagnose and treat these serious diseases, there is a clear need for the identification of additional markers and therapeutic targets. There is a clear need for markers that will facilitate the early-stage detection and treatment of hyperproliferative disorders in humans and other mammals. The identification of factors correlating with hyperproliferative disorders is a prerequisite for the identification of diagnostic markers.

Clearly, hyperproliferative disorders involve unregulated cell division, suggesting an association with aberrant cell cycle regulation. Aberrant signal transduction is associated with aberrant cell cycle regulation and the occurrence of hyperproliferative disorders such as cancer, and DNA damage. In response to DNA damage and replication blocks, cell cycle progression is halted through the control of critical cell cycle regulators. Genomic damage, if left unrepaired, can lead to malignant transformation, or cell death by senescence (aging), necrosis or apoptosis.

For example, the action of steroid hormones generally involves signal transduction cascades involving translocation of hormone receptors from the cytoplasm to the nucleus, thereby effecting many cellular and tissue functions. Progesterone, a 4-pregnene-3,20-dione derived from cholesterol, is a critical component of the female reproductive cycle, wherein oscillations in the serum plasma levels of progesterone during each cycle of ovulation help to mediate biochemical and molecular activity in target tissues and result in anatomical and morphological changes. The molecular target of progesterone is the intracellular progesterone receptor (PR). PR is present in the cytoplasm in a heterocomplex comprising several other proteins and factors termed the PR heterocomplex (PRC). The PR is maintained in an inactive form by molecular chaperones, immunophillins, and heat shock proteins (hsp70, hsp90, hsp27, and p59 (hsp56), p48 and p23; Johnson et al., Mol Cell Biol 14,1956-1963, 1994). Active PR binds progesterone and translocates to the nucleus where it binds as a transcription factor to canonical DNA transcriptional elements present in progesterone-regulated genes. Progesterone-regulated genes have also been implicated in differentiation and in the cell cycle (Moutsatsou and Sekeris, Ann NY Acad Sci 816, 99-115, 1997) and certain cancers.

The assembly of the PRC in vitro involves an ordered interaction between PR and at least eight components. For example, hsp70 binds to the PR and prevents interaction with its ligand; and hsp90 prevents intranuclear translocation by PR in the absence of progesterone (Kang et al., Proc Natl Acad Sci 91, 340-344, 1994). Chemical modification of hsp70 and hsp90 causes release of PR. Other signals may affect the interactions of hsp90 with p23. Arrest of PRC assembly in vitro may be blocked by the selective hsp90 binding agent geldanamycin (GA). Intermediate PR complexes including hsp90 and p23, but do not bind progesterone, are formed in the presence of GA (Smith et al., Mol Cell Biol 15, 6804-6812, 1995). The hsp70 protein binds the mutated tumor-suppressor gene p53 and has been associated with decreased nuclear localization of PR in tissue from node-negative breast tumors (Elledge et al, Cancer Res 54, 3752-3757, 1994).

Ubiquitin-mediated proteoloysis is required for the regulation of many key cellular pathways including the control of cell cycle progression (King et al., Science 274, 1652-1659, 1996; Draetta, Curr, Opin. Cell Biol. 6, 842-846, 1994; Nefsky et al., EMBO J. 15, 1301-1312, 1996), cellular signal transduction (Joanzeiro et al., Science 286, 309-312, 1999; Joanzeiro et al. Cell 102, 549-552, 2000; Zhu et al., Nature 400, 687-693, 1999), DNA damage responses of cells (Beaudenon et al., Mol. Cell. Biol. 19, 6972-6979, 1999) and transcriptional control (Saleh et al., J. Mol. Biol. 282, 933-946, 1998). Although the correct operation of the ubiquitinylation pathway in cells is most likely to play a significant role in ensuring appropriate signal transduction, thereby assisting in protecting against the development of hyperproliferative disorders, no clear mechanism of how this homeostasis is maintained has emerged.

It is generally accepted that proteins having the HECT domain of E6-AP and related proteins (Huibregtse et al., Proc. Natl Acad. Sci. USA 92, 2563-2567. 1995; Schwartz et al., J. Biol. Chem 273, 12148-12154, 1998) form a sub-class of ubiquitin-protein ligases (E3 enzymes) that are involved in the ubiquitinylation cascade that catalyzes the covalent attachment of ubiquitin to a substrate protein, thereby targeting the substrate protein for proteolytic degradation. Unlike the ubiquitin ligases that comprise a RING domain, the enzymes comprising a HECT domain reversibly bind ubiquitin via a conserved cysteine residue within the HECT domain, and directly transfer ubiquitin to the substrate protein (Scheffner et al., Nature 373, 81-83, 1995).

Based upon the presence of a carboxyl terminal HECT domain, a progestin-induced gene that is linked to map position 8q22.3 of the human genome (hereinafter “Edd”) has attracted some interest as a human E3 enzyme (Callaghan et al., Oncogene 17, 3479-3491, 1998). However, the precise cellular function of the protein (EDD) encoded by the Edd gene has not been determined.

The biochemical properties of in vitro translated EDD protein provide evidence that it is a human E3 enzyme (Callaghan et al, Oncogene 17, 3479-3491, 1998) however there are no defined cellular substrates for the protein. The sub-cellular location of the EDD protein is also unknown. Notwithstanding that the progestin-responsiveness of Edd gene expression indicates a general role for EDD protein in progestin-mediated signal transduction pathways or progestin-responsive tumorigenesis, no clear indication of specific proteins that bind to EDD in such pathways has emerged. Nor has there been any indication of specific expression patterns of the Edd gene, or mutations at the Edd locus, that might be involved in mediating such effects. Nor has the Edd gene been implicated in non-progestin mediated cellular effects.

### SUMMARY OF THE INVENTION

In work leading up to the present invention, the inventors sought to elucidate the role of the EDD protein by looking at the expression patterns of the Edd gene in various tumors, and by determining substrates for the EDD protein in cells, and by targeting Edd gene expression in a murine model and by using inhibitory RNA approaches in human cell lines. Surprisingly, the inventors found that elevated Edd gene expression is associated with tumorigenesis in progestin-responsive and progestin-non-responsive tumorigenesis, and that, by reducing or inhibiting Edd gene expression, cellular is inhibited. Accompanying this effect on proliferation is an effect on apoptosis. Effects on cell proliferation and apototic effects on cells as mediated by EDD are closely linked. These data indicate a general utility for the Edd gene and EDD protein in identifying aberrant cell cycle regulation, and modulating the cell cycle, such as, for example, in the treatment of hyperproliferative disorders.

The inventors have also identified many nuclear protein substrates of the EDD protein using yeast two hybrid screens which are involved in progesterone-mediated signal transduction, tumor suppression or DNA damage. Given the key role of the Edd gene expression in the control of cellular proliferation, these data provide highly specific means for identifying aberrant cell cycle regulation and downstream effects such as hyperproliferative disorders. The protein-protein interactions also provide highly specific means for determining compounds that modulate the cell cycle.

Accordingly, one aspect of this invention provides methods for detecting a cancer cell in a subject, said method comprising determining the level of nucleic acid that is linked to map position 8q22.3 of the human genome or an expression product thereof in a sample of said subject, wherein elevated levels of said nucleic acid or said polypeptide are indicative of cancer in the subject.

In one embodiment, the present invention provides a method for detecting a cancer cell in a subject, said method comprising:
(i) determining the level of nucleic acid linked to map position 8q22.3 of the human genome in a test sample from said subject; and
(ii) comparing the level of the nucleic acid at (i) to the level of the nucleic acid in a reference sample from a healthy or normal individual,
   wherein a level of the nucleic acid at (i) that is enhanced in the test sample relative to the reference sample from the normal or healthy individual is indicative of the presence of a cancer cell in said subject.

In an alternative embodiment the invention provides a method for detecting allelic imbalance in a region of the human genome comprising hybridizing a nucleic acid probe or primer to genomic DNA and detecting the hybridization, wherein the probe or primer comprises a nucleotide sequence selected from the group consisting of:
(i) the sequence set forth in SEQ ID NO: 5;
(ii) the sequence set forth in SEQ ID NO: 6;
(iii) the sequence set forth in SEQ ID NO: 7;
(iv) the sequence set forth in SEQ ID NO: 24;
(v) the sequence set forth in SEQ ID NO: 25; and
(vi) the sequence of a nucleic acid fragment produced by amplification using (vi) and (vii) as amplification primers in PCR.

In another embodiment, the present invention provides a method for detecting a cancer cell in a subject, said method comprising:
(i) determining the level of mRNA encoded by nucleic acid linked to map position 8q22.3 of the human genome that is expressed in a test sample from said subject; and
(ii) comparing the level of the mRNA determined at (i) to the level of mRNA encoded by nucleic acid linked to map position 8q22.3 of the human genome that is expressed in a reference sample from a healthy or normal individual,
   wherein a level of the mRNA at (i) that is enhanced in the test sample relative to the reference sample from the normal or healthy individual is indicative of the presence of a cancer cell in said subject.

In another embodiment, the present invention provides a method for diagnosing a cancer or predicting recurrence of a cancer in a subject comprising determining the level of mRNA or protein encoded by nucleic acid linked to map position 8q22.3 of the human genome in a sample of said subject, wherein an elevated level of said mRNA or protein is indicative of relapse of a cancer in said subject.

In one embodiment, the mRNA encoded by a nucleic acid linked to map position 8q22.3 of the human genome encodes an EDD protein. Preferably, the mRNA comprises the sequence set forth in SEQ ID NO: 1 or SEQ ID NO: 3 or a fragment thereof. Even more preferably, the mRNA encodes a protein comprising the sequence set forth in SEQ ID NO: 2 or SEQ ID NO: 4 or a fragment thereof.

In one embodiment the protein encoded by a nucleic acid linked to map position 8q22.3 of the human genome is an EDD protein. Preferably the protein encoded by a nucleic acid linked to map position 8q22.3 of the human genome comprises the sequence set forth in SEQ ID NO: 2 or SEQ ID NO: 4 or a fragment thereof.

A further aspect of the present invention relates to novel nucleic acid probes for detecting a cancer in accordance with the embodiments described herein.

A further aspect of the present invention provides an isolated or recombinant protein complex comprising:
(i) an EDD protein or a portion of an EDD protein sufficient to bind to a protein selected from the group consisting of a protein having tumor suppressor activity, a protein having cell cycle modulatory activity, a protein associated with DNA repair or damage, a nuclear targeting protein, a progesterone receptor protein, and a protein associated with vascularization; and
(ii) a nuclear protein selected from the group consisting of a protein having tumor suppressor activity, a protein having cell cycle modulatory activity, a protein associated with DNA repair or damage, a nuclear targeting protein, a progesterone receptor protein, and a protein associated with vascularization or a portion of said protein sufficient to bind to said EDD protein or said portion of an EDD protein.

Additional embodiments of the present invention provides isolated peptides, polypeptides, antibodies and other ligands that bind to an EDD-containing protein complex of the invention, and kits comprising same for producing the protein complex, or for identifying a modulator of a biological interaction between EDD or a portion of EDD and one or more other polypeptides selected from the group consisting of a protein having tumor suppressor activity, a protein having cell cycle modulatory activity, a protein associated with DNA repair or damage, a nuclear targeting protein, a progesterone receptor protein, and a protein associated with vascularization, or a portion thereof.

Another embodiment of the present invention provides methods for isolating a EDD binding protein or a complex comprising same from a suitable cellular source.

In another embodiment, the invention provides methods for producing a protein complex described herein by recombinant means. For expressing peptides or polypeptides by recombinant means, a protein-encoding nucleotide sequence is placed in operable connection with a promoter or other regulatory sequence capable of regulating expression in a cell-free system or cellular system.

Another embodiment of the present invention provides prognostic and diagnostic methods for determining a predisposition for disease, or a disease state, said methods comprising detecting a protein complex comprising:
(i) an EDD protein; and
(ii) a nuclear protein selected from the group consisting of a protein having tumor suppressor activity, a protein having cell cycle modulatory activity, a protein associated with DNA repair or damage, a nuclear targeting protein, a progesterone receptor protein, and a protein associated with vascularization.

A further embodiment of the present invention provides methods for determining a modulator of the activity, formation or stability of an isolated or recombinant protein complex comprising:
(i) an EDD protein or a portion of an EDD protein sufficient to bind to a protein selected from the group consisting of a protein having tumor suppressor activity, a protein having cell cycle modulatory activity, a protein associated with DNA repair or damage, a nuclear targeting protein, a progesterone receptor protein, and a protein associated with vascularization; and
(ii) a nuclear protein selected from the group consisting of a protein having tumor suppressor activity, a protein having cell cycle modulatory activity, a protein associated with DNA repair or damage, a nuclear targeting protein, a progesterone receptor protein, and a protein associated with vascularization or a portion of said protein sufficient to bind to said EDD protein or said portion of an EDD protein.

In another embodiment, the present invention provides a method for treating a condition associated with elevated expression of an EDD protein in a cell, said method comprising administering an amount of a compound effective to reduce the level of EDD expression or the level of an EDD expression product (e.g. a protein complex comprising an EDD protein) in a cell.

In yet another embodiment, this invention relates to a variant of a gene that is linked to map position 8q22.3 of the human genome and to novel reagents and methods for detecting said variant.

### BRIEF DESCRIPTION OF THE DRAWINGS

FIG. 1. A. Allelic imbalance in several cancer types showing AI at the EDD locus (8q22.3) but not extending continuously to 8q24. Key: ● allelic imbalance, probable allelic imbalance, ◯heterozygote, □uninformative homozygote, gap denotes no data available. Position of polymorphic microsatellites were identified from the Genome Data Base (GDB) (http://gdbwww.gdb.org/). Microsatellites CEDD and 586F18b are encoded in introns of EDD. The EDD gene is located at 8q22.3 (Callaghan et al, Oncogene 14, 3479-3491, 1998) and MYC at 8q24.12 (GDB).

B. Microsatellite analysis of allelic imbalance from normal hepatocellular tissue and hepatocellular carcinoma from patient 8 (FIG. 1A). Allelic imbalance was seen for microsatellites D8S326, CEDD, D8S545 and D8S85. Arrows indicate significant increase (>30%) in proportion of allele indicated.

FIG. 2. Allelic imbalance in ovarian cancers on chromosome 8q22.3-23.3. Cancers are grouped according to histopathology. Overlap indicates mixed histology. ‘Other’ comprises 4 adenomas and a germ cell tumor (case 124). Key: ● allelic imbalance, ◯heterozygote, □uninformative homozygote, gap denotes no data available.

FIG. 3. mRNA expression of EDD in breast cancers, normal breast tissue and breast epithelial cell lines.

(A) Relative mRNA expression of EDD in 41 breast cancers and 14 normal breast samples determined by quantitative RT-PCR. Horizontal line depicts upper limit of normal tissue expression. Insert: Comparison of EDD mRNA expression in 14 breast cancer samples and matched normal breast tissue determined by quantitative RT-PCR.

(B) Expression of EDD mRNA (solid bars) and p53 R2 mRNA (open bars) in breast epithelial cell lines determined by quantitative RT-PCR. Gene copy number (indicated above bars) was determined by quantitative PCR. EDD genomic copy number was determined in MDA-MB-436, MDA-MB-468, BT-20 and BT-483 by FISH (indicated in brackets). 184 is a normal breast epithelial cell lines while all others are breast cancer cell lines (Sutherland et al, Human Cell Culture Vol. II, 79-106, 1999).

FIG. 4 EDD protein expression in breast and ovarian cancers.

Tissues were stained with a polyclonal EDD antibody and counterstained with haematoxylin. (

A) negative control, neural tissue from an EDD-null mouse embryo (neural epithelium [NE], neural mesenchyme [NM]).

(B) positive control, neural tissue from wildtype mouse embryo. (C) normal human breast duct.

(D) human breast carcinoma with high EDD expression.

(E) human serous ovarian cancer with low EDD expression.

(F) human serous ovarian cancer with high EDD expression.

FIG. 5. Structural features of the EDD sequence.

(A) Schematic diagram of EDD and its derivatives used for mammalian expression, yeast two hybrid analysis or in vitro translation. The UBA domain, three putative nuclear localisation sequences (NLS), a HECT domain and domains with homology to N-recognin zinc finger (zf-UBR1) or the carboxy region of polyA-binding protein (PABP-C) are indicated. The positions of potential steroid receptor binding motifs (LXXLL) are indicated by asterisks. Numbers indicate amino acid positions of fragment breakpoints. The conserved cysteine within the HECT domain (Cys 2768) is mutated to alanine (X) in fragments EDDM, EDD3M and EDD5M.

(B) Potential zinc finger in EDD protein. A cysteine-rich domain shows similarity to D. melanogaster Calossin (dCALO) and Arabidopsis thaliana BIG proteins and has a similar arrangement of conserved cysteine and histidine residues (boxed) as the zinc finger region first identified in N-recognin. N-recognin sequences shown are from yeast (scUBR1) and mouse (mUBR1). Identical residues are designated by dark shading and conservative substitutions by lighter shading.

FIG. 6. EDD interacts with importin α5 via two NLSs.

(A) Interaction of EDD with importin α5 in a yeast two-hybrid assay. The entire coding sequence of EDD was fused in-frame with the yeast GAL4 DBD. This construct or control vector pAS2.1 was co-expressed with either control vector (pACT2) or the GAL4 AD-importin a constructs encoding aa 1-538 (Impα) or 229-538 (Impα-C) in diploid yeast strain CG1945/Y187. Protein extracts were prepared from cultures of 6 independent colonies and assayed in duplicate for β-galactosidase activity (expressed as fold increase over pAS2.1 vector control).

(B-C) In vitro interaction of importin α with EDD and mapping of interaction. In vitro translated ³⁵S-labelled EDD (B) or EDD fragments (C) were incubated with a purified GST-importin α5 fusion protein or with GST alone bound to glutathione-Sepharose beads, washed and analysed by SDS-PAGE and autoradiography.

(D) Interaction of EDD with importin α in HEK 293 and T-47D cells. HEK 293 cells were stably transfected with a plasmid encoding full length EDD protein (293/EDD). Extracts from these cells or T-47D cells were subjected to immunoprecipitation with anti-importin α5 antibody (middle panel) or incubated with either GST or GST-importin α5 fusion protein bound to glutathione-Sepharose beads (right panel). Bound proteins from both procedures were separated by SDS-PAGE and western blotted for EDD.

(E) Mapping interaction between importin α and individual NLSs of EDD. In vitro translated ³⁵S-labelled EDD derivatives from the N-terminal region were incubated with GST-importin α5 or with GST alone bound to glutathione-Sepharose beads. Bound EDD was detected by SDSPAGE and autoradiography.

(F) In vitro interaction of importin β with EDD. In vitro translated ³⁵S-labelled EDD and derivates were incubated with GST-importin β fusion protein or with GST alone bound to glutathione-Sepharose beads. Bound EDD was detected by SDS-PAGE and autoradiography. Amounts of bound EDD relative to input are indicated as percentages below.

FIG. 7. EDD is a nuclear protein.

(A) Subcellular localisation of EDD-GFP in mammalian cell lines (×40 magnification). EDD cDNA was fused to the amino-terminus of green fluorescent protein (GFP) and transiently transfected into HEK 293 (left panel) or MCF-7 cells (right panel). Transfected cells are indicated by arrowheads on bright field images. While diffuse cellular staining was observed with GFP alone, strong nuclear GFP fluorescence was observed for EDD-GFP in both cell lines.

(B) Immunostaining of cells with EDD antibody (×40 magnification). Nuclear staining seen for endogenous EDD in HEK 293 cells (left panel) is more intense in HEK 293 cells which overexpress EDD protein (WT30, right panel).

FIG. 8. EDD interacts with PR-B.

(A) Interaction of EDD with PR in T-47D cells. Extracts from T-47D cells were incubated with either GST, GST-PR(AB) or GST-PR(CDE) fusion proteins bound to glutathione-Sepharose beads. Bound proteins were separated by SDS-PAGE and western blotted for EDD.

(B) Mapping interaction between PR and EDD. In vitro translated ³⁵S-labelled EDD derivatives or SRC-1 were incubated with GST-PR(AB) and GST(CDE) fusion protein or with GST alone bound to glutathione-Sepharose beads. PR-bound EDD fragments were analysed by SDSPAGE and autoradiography. Amounts of bound EDD or SRC-1 relative to inputs are indicated as percentages below.

(C) Fine mapping of the interaction between PR(CDE) and the N-terminal region of EDD. In vitro translated ³⁵S-labelled EDD derivatives from the N-terminal region were incubated with GSTPR(CDE) fusion protein or with GST alone bound to glutathione-Sepharose beads. PR(CDE)-bound EDD fragments were analysed by SDS-PAGE and autoradiography.

FIG. 9. Enhancement of nuclear receptor transactivation activity by EDD.

Luciferase activity was corrected for cell number and transfection efficiency where appropriate (see methods) and graphed relative to the value for liganded receptor alone, which was set at 100%.

(A) EDD enhances PR B transactivation activity. Reporter assays were carried out using either HEK 293 (left ) or COS7 (right) cells in the presence of EDD, SRC1 or empty vector, transfection control plasmid (pGFP20) and either 1 nM of the synthetic progestin ORG2058 or equivalent ethanol vehicle (EtOH).

(B) Mutation of the catalytic cysteine of EDD does not alter the effect of EDD on PR transactivation. Reporter assays were carried out using HEK 293 cells in the presence of EDD, EDDM or empty vector and 10 nM ORG2058.

(C) EDD enhances PR reporter gene expression in a dose dependent manner. HEK 293 cells were transfected for a standard reporter assay along with increasing amounts of a constitutive expression vector for either EDD or empty vector (0) in the presence of 1 nM ORG2058. The amount of DNA transfected was normalised to 1.2 μg with empty vector. Cell number was monitored using proliferation assay and transfection efficiency by co-transfection with pRLTK followed by Renilla luciferase assay.

(D) Effect of EDD on response to the synthetic progestin ORG2058. HEK 293 cells were transfected for reporter assay along with a transfection control plasmid (pRL-TK). Cells were harvested for luciferase assay following 24 h treatment with increasing concentrations of ORG2058.

(E) Enhancement of VDR reporter gene expression by EDD. HEK 293 cells were transfected with a constitutive expression vector for VDR and a VDRE-containing luciferase reporter vector along with a constitutive expression vector for EDD or empty vector and a transfection control plasmid (pRL-TK). Cells were harvested for luciferase assay following 24 h treatment with 10 nM 1,25-dihydroxyvitamin D₃.

(F) EDD does not enhance ER reporter gene expression. HEK 293 cells were transfected with a constitutive expression vector for ER and an ERE-containing luciferase reporter vector along with either a constitutive expression vector for EDD, SRC1 or empty vector and a transfection control plasmid (pGFP20). Cells were harvested for luciferase assay following 24 h treatment with 100 nM 17 β-estradiol.

FIG. 10. Interaction of EDD and CIB and the effect of DNA damage.

(A) Interaction of EDD with calcium-integrin binding protein (CIB) in a yeast two-hybrid assay. The entire coding sequence of EDD was fused in-frame with the yeast GAL4 DBD. This construct or control vector pAS2.1 was co-expressed with either control vector (pACT2) or GAL4 AD-CIB in diploid yeast strain CG1945/Y187.

(B) In vitro interaction of CIB with EDD. In vitro translated ³⁵S-labelled EDD was incubated with GST-CIB fusion protein or with GST alone bound to glutathione-Sepharose beads. Bound EDD was analysed by SDS-PAGE and autoradiography.

(C) Mapping interaction between CIB and EDD. In vitro translated ³⁵S-labelled EDD derivatives were incubated with GST-CIB fusion or with GST alone bound to glutathione-Sepharose beads. Bound EDD fragments were analysed by SDS-PAGE and autoradiography.

(D) Interaction of EDD and CIB in HEK 293 and MCF-7 cells. Left, HEK 293 cells overexpressing mutant EDD were transfected with a plasmid encoding Flag-tagged CIB or empty vector (vec). Extracts from these cells were subjected to immunoprecipitation using anti-FLAG Ab M2. Right, nuclear extracts prepared from MCF-7 cells following treatment with DNA damage agents phleomycin (Phleo) or hydroxyurea (HU) were incubated with either GST or GST-CIB fusion protein bound to glutathione-Sepharose beads. Bound proteins from both procedures were analysed by SDS-PAGE and western blotted for EDD and amounts bound are indicated as percentages relative to input.

(E) Potential regulation of CIB by the proteasome. HEK 293 cells were treated is with the proteasome inhibitor MG132 (20 μM) or vehicle (DMSO) for six hours and whole cell extracts analysed by SDS PAGE. Proteins were transferred to nitrocellulose and western blotted for importin α5, CIB and the proteasomal target protein, p27.

FIG. 11

(A) Schematic diagram of EDD and chk2 and their derivatives used in binding assays. For EDD, the UBA (ubiquitin-associated) domain, three putative nuclear localisation sequences (NLS), a HECT (homologous to E6-AP carboxy terminus) domain and domains with homology to N-recognin zinc finger (zf-UBR1) or the carboxy region of polyA-binding protein (PABP-C) are indicated. The positions of potential steroid receptor binding motifs (LXXLL) are indicated by asterisks. Numbers indicate amino acid positions of fragment breakpoints. The conserved cysteine within the HECT domain (Cys 2768) is mutated to alanine (X) in fragments EDDM and EDD3M. For chk2, the SQ/TQ domain rich in ATM family kinase sites, the forkhead associated (FHA) domain and kinase domain are indicated along with key residues for chk2 function. Domain boundaries are also indicated as numbers below the diagram. Also indicated is the fragment of chk2 used in pull down assays (GSTchk2-N).

(B) EDD interacts with chk2-N in nuclear extracts. Nuclear extracts from MCF-7 cells were incubated with either GST or GSTchk2-N fusion protein bound to glutathione-Sepharose beads. Following extensive washing, bound proteins were separated by SDS-PAGE and western blotted for EDD.

(C) EDD and chk2 associate in vivo. Cell lysates from HEK 293 or MCF-7 cells were subjected to immunoprecipitation with polyclonal chk2 antibody (N17). Following precipitation of immune complexes and extensive washing, bound proteins were separated by SDS-PAGE and western blotted for EDD.

FIG. 12

EDD interacts with chk2 FHA domain.

(A) In vitro translated ³⁵S-labelled EDD derivative EDDF5 (aa 889-2799) was incubated with GSTchk2-N or GSTchk2-N derivatives containing R117A or I157T substitutions, or with GST alone bound to glutathione-Sepharose beads. Bound EDD was detected by SDS-PAGE and autoradiography.

(B) Nuclear extracts from MCF-7 cells were incubated with GSTchk2-N or a GSTchk2-N derivative containing an R117A substitution, or with GST alone bound to glutathione-Sepharose beads. Bound proteins were separated by SDS-PAGE and western blotted for EDD.

(C) EDD is phosphorylated in vivo. HEK293 cells stably transfected with Ecdysone receptor were transfected with a vector for inducible expression of Flag-tagged EDD. Expression of EDD was induced (+) by addition of ponasterone for 24 h. Medium was replaced with phosphate free medium containing ³²P-labelled orthophosphate and labelling of cellular proteins allowed to proceed. Lysates were made from cells expressing (+) or not expressing (−) Flag-tagged EDD and immunoprecipitation carried out using anti-Flag antisera. Radiolabelled (ie phosphorylated) EDD was detected by SDS-PAGE and autoradiography (upper panel). In the lower panel, HEK293 or MCF-7 extracts were incubated in the presence (+) or absence (−) of lambda protein phosphatase. Samples were separated by SDS-PAGE on 4.2% gels and western blotted for EDD. Removal of phosphates was indicated by a shift in the mobility of the EDD protein.

(D) The phosphopeptide-binding interface of the chk2 FHA domain is required for EDD binding. In vitro translated ³⁵S-labelled EDD derivative EDDF5 (aa 889-2799) was incubated with GSTchk2-N or GSTchk2-N derivatives containing R117A or I157T substitutions, or with GST alone bound to glutathione-Sepharose beads. The incubations with GSTchk2-N or GSTchk2-N(I157T) were carried out in the presence or absence of an FHA-binding phosphopeptide at 0, 80 or 150 μM. Bound EDD was detected by SDS-PAGE and autoradiography.

FIG. 13

Mapping the interaction between EDD and chk2.

(A) In vitro translated ³⁵S-labelled EDD derivatives were incubated with GSTchk2-N or with GST alone bound to glutathione-Sepharose beads. Bound EDD was detected by SDS-PAGE and autoradiography.

(B) In vitro translated ³⁵S-labelled EDD derivatives of EDDF5 (EDDF6 and EDDF7) were incubated with GSTchk2-N or GSTchk2-N derivatives containing R117A or I157T substitutions, or with GST alone bound to glutathione-Sepharose beads. Bound EDD was detected by SDS-PAGE and autoradiography. Fusion protein additions were monitored by blotting for GST.

FIG. 14

(A) Nuclear EDD protein levels and CHK2 T68 phosphorylation before and after irradiation. MCF-7 cells were exposed to 12 Gy IR and allowed to recover for 1, 4 or 12 h. Nuclear extracts were prepared from control and treated cells and analysed by western blotting for EDD, phosphorylated CHK2 (P-T68) or pRB (loading control).

(B) Decreased association between EDD and CHK2 following radiomimetic treatment of cells. Nuclear extracts from MCF-7 cells that had been cultured in the presence of the radiomimetic phleomycin (100 μg per ml) or from control MCF-7 cells were immunoprecipitated with polyclonal CHK2 antibody (N17) or incubated with goat IgG as a negative control. Following precipitation of immune complexes and extensive washing, bound proteins were separated by SDS-PAGE and western blofted for EDD or CHK2.

(C) Nuclear extracts from MCF-7 cells that had been cultured in the presence of phleomycin (100 μg per ml) or from control MCF-7 cells were incubated with GSTCHK2-N or with GST alone bound to glutathione-Sepharose beads. Bound proteins were separated by SDS-PAGE and immunoblotted for EDD and GST.

(D) Nuclear extracts from MCF-7 cells that had been cultured in the presence of phleomycin (100 μg per ml) for 1 h followed by 1 h or 3 h recovery, or from control MCF-7 cells, were incubated with CHK2 antibody (N17). Following precipitation of immune complexes and extensive washing, bound proteins were separated by SDS-PAGE and western blofted for EDD or CHK2.

(E) Dissociation of EDD and CHK2 is not dependent on ATM kinase. Nuclear extracts from MCF-7 cells that had been cultured for 3 h in the presence of the radiomimetic phleomycin (100 μg per ml) or from control MCF-7 cells, in the presence or absence of wortmannin (10 μM), were incubated with GSTCHK2-N or with GST alone bound to glutathione-Sepharose beads. Bound proteins were separated by SDS-PAGE and immunoblotted for EDD and GST. Input lysates were immunoblotted for EDD and active CHK2 (anti P-T68).

(F) Immunoprecipitation with CHK2 antibody from nuclear extracts of cells deficient in ATM (FTpEBS7) and a matched ATM-complemented cell line (FTYZ5). Cells were incubated in the presence or absence of phleomycin (20 μg per ml) for 4 hours, ensuring T68 phosphorylation in an ATM-dependent fashion (lower panel).

FIG. 15

(A) MCF-7 cells were transfected with short interfering RNAs directed against GFP or EDD for 96 h. Cells were then exposed to 12 Gy ionizing radiation (IR) and harvested following 90 min recovery at 37° C. Equal amounts of protein from whole cell lysates were separated by SDS-PAGE and analysed by immunoblofting for EDD, activated CHK2 (P-T68) and total CHK2.

(B) Kinase assay of CHK2 immunoprecipitated from irradiated cell lysates using GST-cdc25C subfragment as a substrate. MCF7 control cells or cells depleted of EDD (EDD siRNA) were subjected to 4 Gy ionising radiation (IR) and allowed to recover for 15 or 60 minutes before harvesting. Incorporation of 32-P into substrate or CHK2 was detected by autoradiography. The amounts of immunoprecipitated CHK2 and the efficiency of phosphorylation of CHK2 on threonine 68 were monitored by western blotting.

FIG. 16

The phosphopeptide-binding interface of the CHK2 FHA domain is required for EDD binding.

(A) CHK2 FHA domain binds EDD. In vitro translated (IVT) ³⁵S-labelled EDD derivatives EDDF5 (aa 889-2799) and EDDF6 (aa 889-2526) (top panel) or MCF7 whole cell extracts (lower panel) were incubated with GSTCHK2-N (WT) or GSTCHK2-N derivatives containing R117A or I157T substitutions, or with GST alone bound to glutathione-Sepharose beads. Bound EDD was detected by SDS-PAGE and autoradiography (top panel) or western blotting (lower panel). Fusion protein additions were monitored by Coomassie staining.

(B) Dephosphorylation of EDD prevents CHK2 association. Pull downs of IVT EDD were performed as described in (A) except that-where indicated IVT EDD was incubated with lambda protein phosphatase (PPase) prior to incubation with the GSTCHK2-N fusion.

(C) In vitro translated ³⁵S-labelled EDD derivatives EDDF5 and EDDF6 were incubated with GSTCHK2-N (WT) or GSTCHK2-N (I157T) bound to glutathione-Sepharose beads in the presence or absence of FHA-binding phosphopeptide at 0, 20, 50 or 100 μM. Bound EDD was detected by SDS-PAGE and autoradiography.

FIG. 17

(A) EDD and BRCA2 interact in cells. Extracts from HEK293 cells expressing Histagged EDD were subjected to immunoprecipitation with EDD antibody (AbPEP1). Following precipitation of immune complexes and extensive washing, bound proteins were separated by SDS-PAGE and western blotted for EDD (left panel) and BRCA2 (right panel).

(B) Interactions between EDD, BRCA2 and chk2 are modified in response to DNA damaging agents. Nuclear extracts from MCF-7 cells that had been cultured in the presence of the radiomimetic phleomycin or the UV mimic hydroxyurea (HU), or from control MCF-7 cells, were subjected to immunoprecipitation with polyclonal chk2 antibody (N17). Following precipitation of immune complexes and extensive washing, bound proteins were separated by SDS-PAGE and western blotted for EDD, BRCA2 or chk2.

FIG. 18. Targeted disruption of mouse Edd.

(A) Structure of the wild-type Edd locus (top), targeting vector (middle) and the mutated locus following homologous recombination (bottom). Edd exon 1 is indicated as a black box with the position of the ATG codon shown. BamH1 restriction sites are denoted by B. Genotyping was performed by PCR using primers, 1, 2 and 3, (arrowheads), and by Southern blot analysis with a 3′ probe as shown (probe). Expected sizes of PCR products and BamHl fragments that hybridise with the probe on Southern analysis are indicated.

(B) Southern blot and PCR analysis of genomic DNA from targeted ES cell clones. Genomic DNA from four neomycin resistant ES cell clones, 1B2, 3D5, 5C6 and 8E7, was digested with BamHl and hybridised with the 3′ probe. The 6.0 kb fragment corresponding to the wild-type (WT) allele (top) and the 4.2 kb fragment corresponding to the mutated (KO) allele (bottom) are indicated. Right panel: PCR genotype analysis of E10.5 embryos. DNA samples were subjected to PCR using the primers 1, 2 and 3. PCR amplification of the WT allele by primers 1 and 2 produces a 600 bp fragment (top) and of the KO allele by primers 2 and 3 produces a 440 bp fragment (bottom).

(C) Northern Blot analysis of tissues from WT and Edd^{±} mice. Total RNA was extracted and hybridised to a ³²P-labelled mouse Edd cDNA probe. Densitometry was performed and values corrected for loading by comparison to the blot after reprobing with GAPDH cDNA.

(D) Western blot analysis and IHC showing EDD expression in wild-type (+/+), heterozygote (±) and knockout (−/−) tissues. EDD western blots were performed on lysates from testes of adult WT (+/+) and Edd^{±} (±) mice and from E10.5 WT, Edd^{±} and Edd^{-/-} (−/−) embryos.

(E) Immunohistochemistry was performed on E9.5 wild-type (left panel) and E9.5 knockout (−/−, right panel) embryo sections with a region of neural epithelium shown. Immunohistochemstry was performed with an anti-EDD antibody.

FIG. 19. Morphology of wild-type and knockout (Edd^{-/-}) embryos at E7.5 -E10.5. Photographs showing freshly dissected WT embryos (+/+) and Edd^{-/-} embryos (−/−). As early as E7.5, Edd^{-/-} embryos display slightly delayed development while by E8.5, clear growth retardation is obvious in Edd^{-/-} embryos compared to WT. From E9.5 onwards the most obvious developmental defect is the small size of Edd^{-/-} embryos and the absence of turning which occurs in WT embryos around E9. In addition, many Edd^{-/-} embryos display a bulbous allantois (a) indicating failure of placentation. Many Edd^{-/-} embryos were also observed with pericardial effusion (p).

FIG. 20. Edd expression in developing mouse embryo. Immunohistochemical analysis of Edd expression in wt d10.5 embryo. Inset shows predominantly nuclear expression under higher magnification.

FIG. 21. Cell proliferation in WT and Edd^{-/-} embryos at E8.5 -E10.5.

(A) BrdU incorporation was used to measure cell proliferation in WT (+/+) and Edd^{-/-} (−/−) embryos and BrdU positive cells were detected by immunohistochemistry. Cells positive for BrdU are darkly stained.

(B) Quantification of prolioferating cells in Edd^{-/-} embryos at E8.5 -E10.5. While there are equivalent numbers of proliferating cells between Edd^{-/-} and WT embryos at E8.5, by E9.5 the majority of cells in Edd^{-/-} embryos have stopped proliferating and do not incorporate BrdU. Graph shows mean number of proliferating cells (% total±standard error).

FIG. 22. Apoptosis in WT and Edd−/− embryos at E8.5 -E10.5.

(A) Apoptosis was measured in embryo sections using the TUNEL assay with TUNEL positive nuclei stained brown. At E8.5, similar levels of staining can be seen in both WT (+/+) and Edd^{-/-} (−/−) embryos. At E9.5 and 10.5, Edd^{-/-} embryos show dramatically increased levels of TUNEL staining compared to WT embryos which have very few TUNEL stained cells.

(B) Active Caspase-3 staining was also performed on embryo sections to confirm TUNEL results. Percentage of caspase positive cells are shown in the corner of each panel. Similarly to TUNEL results, levels of staining in WT (+/+) and Edd^{-/-} embryos (−/−) at E8.5 are very similar. However, by E9.5 a large increase in the level of caspase-3 mediated apoptosis is observed in Edd^{-/-} embryos.

FIG. 23. Defective vascularisation in Edd^{-/-} yolk sacs. (A) Morphology of E9.5 -E10.5 WT (+/+) and Edd^{-/-} (−/−) yolk sacs. Edd^{-/-} yolk sacs display significantly less vascularisation than their WT littermates suggesting a defect in yolk sac circulation. In addition blood pooling can be seen within Edd^{-/-} embryos at E10.5 (b);

(B) High magnification view of Edd^{-/-} and WT yolk sacs at E10.5. WT yolk sacs contain large and well organised vessels (v) however, while Edd^{-/-} yolk sacs do contain some vessels (v), these appear small and disorganised suggesting defective vascular development in the absence of EDD.

FIG. 24. High magnification view of histological sections from WT and Edd^{-/-} yolk sacs at E9.5. Distinct vascular channels containing blood cells (b) are visible in WT yolk sac, whereas EDD-null yolk sacs display enlarged channels with unusual separation of mesoderm (m) and endoderm (e) and few blood cells.

FIG. 25. Western blot analysis of EDD protein expression after anti-EDD (R) or anti-GFP (C) (control) small interfering RNA transfection in MCF-7 and HEK-293 cell lines. Time after transfection is shown in hours at the bottom of the figure.

FIG. 26 Cell morphology after RNA interference. HMEC 184 cells are shown 5 days after RNA interference (left) control cells and (right) cells transfected with EDD siRNA. Cell shape has altered after depletion of EDD, cells make fewer contacts and cell organisation is disturbed. (Original magnification 100×)

FIG. 27. Immunofluorescence microscopy of β-catenin in HMEC 184 cells. Whereas β-catenin staining in control cells (left) was even along cell-cell contacts, cells transfected with EDD siRNA showed more patchy staining and decreased levels of β-catenin (right). Furthermore, a reduction in the number of cell-cell contacts was observed in cells transfected with EDD siRNA.

FIG. 28. Relocalization of β catenin after EDD RNAi transfection. Immunofluorescence microscopy of β-catenin in HMEC 184 cells shows that EDD RNAi causes β-catenin to move from the cell periphery (left, control cells) to the cell nucleus (right).

FIG. 29. Immunofluorescence microscopy of actin in HMEC 184 cells. Actin filaments (in control cells (left) were coordinated and continuously organised from cell to cell. Cells transfected with EDD siRNA (right) showed disorganised actin filaments, where the actin from one cell was not connected to the actin of its neighbouring cells.

### DETAILED DESCRIPTION OF THE INVENTION

Nucleic Acid-Based Diagnostics and Novel Nucleic Acids

One embodiment of this invention provides methods for detecting a cancer cell in a subject, said method comprising determining the level of nucleic acid that is linked to map position 8q22.3 of the human genome or an expression product thereof in a sample of said subject, wherein elevated levels of said nucleic acid or said polypeptide are indicative of cancer in the subject.

In the present context, the term “cancer cell” includes any biological specimen or sample comprising a cancer cell irrespective of its degree of isolation or purity, such as, for example, tissues, organs, cell lines, bodily fluids, or histology specimens that comprise a cell in the early stages of transformation or having been transformed. Bodily fluids shall be taken to include whole blood, serum, peripheral blood mononuclear cells (PBMC), or buffy coat fraction.

The isolated nucleic acid inked to map position 8q22.3 of the human genome or an expression product thereof is present at elevated levels in cancer cells compared to non-cancer cells.

The definition of “cancer cell” is not to be limited by the stage of a cancer in the subject from which said cancer cell is derived (ie. whether or not the patient is in remission or undergoing disease recurrence or whether or not the cancer is a primary tumor or the consequence of metastases). Nor is the term “cancer cell” to be limited by the stage of the cell cycle of said cancer cell.

In one embodiment, the cancer cell is epithelial in origin (i.e. a carcinoma).

In another embodiment, the cancer cell is from a cancer selected from the group consisting of ovarian cancer, melanoma, metastatic melanoma, squamous cell carcinoma of the head and neck, squamous cell carcinoma of the tongue, hepatocellular carcinoma, breast cancer, a metastases of ovarian cancer, a metastases of melanoma, a metastases of metastatic melanoma, a metastases of squamous cell carcinoma of the head and neck, a metastases of squamous cell carcinoma of the tongue, a metastases of hepatocellular carcinoma and a metastases of breast cancer.

As used herein, the term “ovarian cancer” shall be taken to refer any one or more of a number of cancers of epithelial origin, such as, for example, serous, mucinous, endometrioid, clear cell, papillary serous, Brenner cell or undifferentiated adenocarcinoma. The term “breast cancer” shall be taken to refer to a ductal carcinoma or lobular carcinoma. The term “hepatocellular carcinoma” will be understood to mean any carcinoma arising from the hepatocytes, as distinct from other types of hepatic cancer that may consist of liver metastases. The terms “melanoma” and “squamous cell carcinoma” will be understood to be epithelial skin cancers of the melanocytes and squamous cells, respectively. “Metastatic melanoma” is the most advanced stage of melanoma arising in the melanocytes and metastasizing in the lymph nodes and other organs of the body.

This embodiment of the invention is predicated on the finding by the present inventors that there is considerable allelic imbalance in several tumor types in the vicinity of the region of the human genome that maps to position 8q22.3. In particular, allelic imbalance in this region of the human genome was found by the inventors in 14 of 37 tumors, said allelic imbalance comprising discrete regions of imbalance comprising 8q22.3 and more extensive 8q aberrations. Allelic imbalance was found by the inventors to be frequent in cases of ovarian cancer (e.g. serous subtype), hepatocellular carcinoma, squamous cell carcinoma of the tongue, breast cancer and metastatic melanoma. Using quantitative RT-PCR, the inventors also found that the expression levels of EDD-encoding mRNA are frequently elevated in cancers such as breast cancer. In the case of breast cancer, elevated expression of the Edd gene was associated with amplification of the Edd locus. A closely linked gene in this region of the genome, i.e. encoding p53 ribonucleotide reductase (p53R2), was also shown by the inventors to be overexpressed and amplified in cancer cell lines. Accordingly, this embodiment of the invention is not limited to the detection of EDD-encoding nucleic acid.

As used herein, the term “map position 8q22.3 of the human genome” shall be taken to refer to the region of the human genome that comprises a genomic gene encoding an EDD protein and preferably having a centromeric orientation on chromosome 8 with its first exon position at about 8q23. Those skilled in the art will be aware that a “genomic gene” includes both protein-encoding regions (i.e. exons) and non-coding regions (i.e. 5′-upstream regulatory regions such as the promoter and 5′-untranslated region, intervening sequences or introns, and 3′-untranslated region). Accordingly, a genomic gene encoding an EDD protein includes all such features and not merely the protein-encoding portion thereof.

By “EDD protein” is meant a polypeptide that comprises an amino acid sequence having at least about 80% identity to the sequence set forth herein as SEQ ID Nos: 2 or 4. For the purposes of nomenclature, the amino acid sequence set forth in SEQ ID NO: 2 relates to a first EDD protein encoded by the nucleotide sequence of SEQ ID NO: 1 as disclosed in published International Patent Application No. PCT/AU98/00280. The sequence set forth in SEQ ID NO: 4 relates to a novel EDD polypeptide encoded by a splice variant wherein 18 nucleotides of SEQ ID NO: 1 have been deleted. Accordingly, the amino acid sequence of SEQ ID No: 4 differs by the deletion of six amino acids comprising the sequence VLLLPL from SEQ ID NO: 2. Preferably, the percentage identity to SEQ ID NO: 2 or 4 is at least about 90%, more preferably at least about 95%, or at least about 99%. In determining whether or not two amino acid sequences fall within these defined percentage identity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP program of the Computer Genetics Group, Inc., University Research Park, Madison, Wis., United States of America (Devereaux et al, Nucl. Acids Res. 12, 387-395,1984), which utilizes the algorithm of Needleman and Wunsch J. Mol. Biol. 48, 443-453, 1970, or alternatively, the CLUSTAL W algorithm of Thompson et al., Nucl. Acids Res. 22, 4673-4680, 1994, for multiple alignments, to maximize the number of identicallsimilar amino acids and to minimize the number and/or length of sequence gaps in the alignment.

It will be apparent from the preceding description that nucleic acid that is “linked to” map position 8q22.3 of the human genome will therefore be sufficiently close to a genomic gene encoding an EDD protein for the frequency of recombination between said nucleic acid and said genomic gene to indicate linkage. As will be known to those skilled in the art, the frequency of recombination between two markers on DNA will increase as the markers are spaced further apart until the association between the markers in a segregating population is random, at which point they are considered to be unlinked.

In one embodiment, the nucleic acid that is linked to map position 8q22.3 will be tightly linked such that there is only a low degree of recombination between the nucleic acid and the genomic gene encoding an EDD polypepude. Preferably, the nucleic acid will itself map to a region of the human chromosome between 8q22.3 and 8q24.13, at least comprising the genomic Edd and p53R2 genes or a portion thereof.

Unless specifically stated otherwise, the expression product of any nucleic acid that is linked to map position 8q22.3 of the human genome is useful for the diagnosis of cancer within the context of the present invention, including the expression products of an Edd gene, or the expression product of a p53R2 gene. As used herein, the term “expression product” shall be taken to refer to any transcription product of a genomic gene, such as unprocessed or processed mRNA including a splice variant, or any translation product encoded by a genomic gene, such as a precursor polypeptide, processed polypeptide or a complex involving said polypeptide. For example, a protein-protein complex or DNA-protein complex comprising an EDD polypeptide is clearly within the scope of the term “expression product of an Edd gene” or similar term as used herein. Similarly, a protein-protein complex or DNA-protein complex comprising a p53R2 polypeptide is clearly within the scope of the term “expression product of an p53R2 gene” or similar term as used herein.

It will be apparent from the preceding discussion that the diagnostic methods provided by the present invention involve a degree of quantification to determine, on the one hand, the level of nucleic acid linked to map position 8q22.3 of the human genome in tissue that is suspected of comprising a cancer cell, or, on the other hand, the level of an expression product of said nucleic acid. Such quantification can be readily provided by the inclusion of appropriate reference samples in the assays described below, derived from healthy or normal individuals.

Accordingly, in one embodiment, the reference sample comprises cells or tissue from the same subject taken at a time point when the individual was healthy or in remission from disease. In yet another embodiment, the reference sample comprises cells or tissues from a healthy or normal individual. In accordance with both of these embodiments, the reference sample and the test sample are both processed, albeit not necessarily at the same time, and data obtained for both samples are compared.

Alternatively, if reference samples are not included in each assay conducted, the reference sample may be derived from an established data set that has been generated from healthy or normal individuals. Accordingly, in one embodiment, the reference sample comprises data from a sample population study of healthy individuals, such as, for example, statistically significant data for the healthy range of the integer being tested. In accordance with this embodiment, the comparison of reference and test sample is performed following the analysis of the test sample and comprises a comparison of data obtained for the test sample to data obtained for the sample population.

In the present context, the term “healthy individual” shall be taken to mean an individual who is known not to suffer from cancer, such knowledge being derived from clinical data on the individual, including, but not limited to, a different cancer assay to that described herein. As the present invention is particularly useful for the early detection of cancer, it is preferred that the healthy individual is asymptomatic with respect to the early symptoms associated with a particular cancer. In the case of ovarian cancer, early detection using well-known procedures is difficult, however reduced urinary frequency, rectal pressure, and abdominal bloating and swelling, are associated with the disease in its early stages, and, as a consequence, healthy individuals should not have any of these symptoms. It is also preferred for such “healthy subjects” to not have a large number of risk factors associated with these diseases. Indicators of the early stages of primary breast cancer or a susceptibility for developing breast cancer include, for example, familial history, atypical hyperplasia, the occurrence of benign conditions of the breast, enhanced breast density particularly in women aged 45 years and older, radiation exposure, and abnormal breast appearance. Indicators of the early stages of primary hepatocellular carcinoma or a susceptibility for the disease include, for example, liver cirrhosis, chronic hepatitis B or hepatitis C infection, a diet high in aflatoxins, adenomatous hyperplasia, abdominal (hepatic) pain or swelling, ascites, enlargement of the spleen, hemochromatosis, alpha-1-antitrypsin deficiency, glycogen storage disease, and tyrosinemia. Indicators of the early stages of primary melanoma or squamous cell carcinoma or a susceptibility for these diseases include, for example, changes to the appearance of the skin, especially in association with prolonged exposure to the sun or radiation damage, a history of smoking or chewing tobacco, actinic keratosis, and leukoplakia. Clearly, subjects suffering from later symptoms associated with any one or more of these cancers, such as, for example, metastases in the skin, oral cavity, pharynx, omentum, abdomen, abdominal fluid, lymph nodes, lung, liver, brain, or bone, and subjects suffering from spinal cord compression, abdominal pain or swelling, elevated calcium level, elevated serum alpha-fetoprotein level, changes in BRCA1 or BRCA2 genes or gene expression, chronic pain, or pleural effusion, should also be avoided from the “healthy individual” data set.

The term “normal individual” shall be taken to mean an individual having a normal level of an Edd gene expression product in a particular sample derived from said individual. As will be known to those skilled in the art, data obtained from a sufficiently large sample of the population will normalize, allowing the generation of a data set for determining the average level of a particular parameter. Accordingly, the level of expression of an Edd gene product can be determined for any population of individuals, and for any sample derived from said individual, for subsequent comparison to levels of the expression product determined for a sample being assayed. Where such normalized data sets are relied upon, internal controls are preferably included in each assay conducted to control for variation.

In one embodiment, the present invention provides a method for detecting a cancer cell in a subject, said method comprising:
(i) determining the level of nucleic acid linked to map position 8q22.3 of the human genome in a test sample from said subject; and
(ii) comparing the level of the nucleic acid at (i) to the level of the nucleic acid in a reference sample from a healthy or normal individual,
   wherein a level of the nucleic acid at (i) that is enhanced in the test sample relative to the reference sample from the normal or healthy individual is indicative of the presence of a cancer cell in said subject.

Preferably, the sample comprises cells from a tissue or tissue selected from the group consisting of skin, an oral cavity tissue, breast, liver, spleen, ovary, prostate, kidney, uterus, placenta, cervix, omentum, rectum, brain, bone, lung, lymph, urine, semen, blood, abdominal fluid, and serum. Cell preparations or nucleic acid preparation derived from such tissues or cells are not to be excluded. The sample can be prepared on a solid matrix for histological analyses, or alternatively, in a suitable solution such as, for example, an extraction buffer or suspension buffer, and the present invention clearly extends to the testing of biological solutions thus prepared.

Preferably, the nucleic acid that is determined according to this embodiment is genomic DNA. This embodiment of the invention is therefore particularly suited to determining allelic imbalance in this region of the human genome as a diagnostic for cancer.

To determine the level of the nucleic acid, a variety protocols that are well known in the art can be utilized, such as, for example, in situ hybridization, microsatellite analysis, and microarray technology, such as, for example, using tissue microarrays probed with nucleic acid probes, or nucleic acid microarrays (ie. genomic DNA microarrays or amplified DNA microarrays) probed with nucleic acid probes. All such assay formats are encompassed by the present invention. For high throughput screening of large numbers of samples, such as, for example, public health screening of subjects, particularly human subjects, having a higher risk of developing cancer, microarray technology is a preferred assay format. As will be understood by those skilled in the art, nucleic acid hybridization-based or amplification-based approaches such as, for example, microsatellite analysis, are readily adaptable to microarray technology.

In a preferred embodiment, the level of nucleic acid is determined by hybridizing a nucleic acid probe to genomic DNA encoding an EDD protein in the test sample under at least low stringency hybridization conditions and detecting the hybridization using a detection means. Similarly, the level of genomic DNA encoding an EDD protein in the reference sample from the healthy or normal individual is preferably determined by hybridizing the probe to genomic EDD-encoding DNA in said reference sample under at least low stringency hybridization conditions and detecting the hybridization using a detection means.

For nucleic acid hybridization-based approaches, shorter probes are hybridized at lower stringency hybridization (ie. reduced temperature and/or higher salt concentration and/or higher detergent concentration) than longer nucleic acid probes. Generally, hybridization is carried out well below the calculated melting temperature (Tm) of a DNA duplex comprising the probe. For example, the oligonucleotide probes exemplified herein have calculated Tm values in the range of about 55° C. to about 60° C., suggesting that hybridization involving such probes should be carried out at a temperature in the range of ambient temperature to about 45° C., and more preferably between about 40° C. to about 45° C. (ie. low stringency to moderate stringency conditions). This contrasts with standard hybridization temperatures of about 65° C. for nucleic acid probes of about 100 nucleotides or longer (ie. moderate to high stringency hybridization conditions).

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, or the type of hybridization probe used.

In one embodiment, the sample or the probe is immobilized on a solid matrix or surface (e.g., nitrocellulose). For high throughput screening, the sample or probe will generally comprise an array of nucleic acids on glass or other solid matrix, such as, for example, as described in WO 96/17958. Techniques for producing high density arrays are described, for example, by Fodor et al., Science 767-773, 1991, and in U.S. Pat. No. 5,143,854. Typical protocols for other assay formats can be found, for example in Current Protocols In Molecular Biology, Unit 2 (Northern Bloffing), Unit 4 (Southern Blotting), and Unit 18 (PCR Analysis), Frederick M. Ausubul et al. (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), or in situ polymerase chain reaction.

The probe can be labelled with a reporter molecule capable of producing an identifiable signal (e.g., a radioisotope such as ³²P or ³⁵S, or a fluorescent or biotinylated molecule, or a coloured dye e.g. TAMRA, FAM, ROC, etc). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about 20 contiguous nucleotides in length is hybridized to genomic DNA and nucleic acid copies of the template are enzymically-amplified.

Those skilled in the art will be aware that there must be a sufficiently high percentage of nucleotide sequence identity between the probes and the nucleotide sequence of the sample template molecule for hybridization to occur. As stated previously, the stringency conditions can be selected to promote hybridization.

In one format, PCR provides for the hybridization of non-complementary probes to different strands of a double-stranded nucleic acid template molecule (ie. a DNA/DNA template), such that the hybridized probes are positioned to facilitate the 5′-to 3′ synthesis of nucleic acid in the intervening region, under the control of a thermostable DNA polymerase enzyme. In accordance with this embodiment, one sense probe and one antisense probe as described herein is used.

Variations of the embodiments described herein are described in detail by McPherson et al., PCR: A Practical Approach. (series eds, D. Rickwood and B. D. Hames), IRL Press Limited, Oxford. pp 1-253, 1991.

In one embodiment, the probe detects non-coding nucleic acid (i.e. an intron, 5′-upstream region or 3′-untranslated region). By way of exemplification, the nucleotide sequences set forth in SEQ ID NOs: 5 and 6, are used to amplify nucleic acid comprising a microsatellite designated “CEDD” that successfully detects allelic imbalance at the region of the human genome between map positions 8q22.3 and 8q24.13 associated with ovarian cancer, hepatocellular carcinoma, breast cancer, squamous cell carcinoma and metastatic melanoma. The microsatellite CEDD also successfully distinguishes serous ovarian cancer from other ovarian cancers of epithelial origin.

The nucleotide sequence of the amplification product comprising the microsatellite CEDD is set forth in SEQ ID NO: 7 and, as will be known to those skilled in the art this amplified fragment is also useful for directly hybridizing to genomic DNA of a subject in the assay formats described herein for the purpose of diagnosing the cancers supra, particularly ovarian cancer.

In other exemplified embodiments described herein, the present invention clearly provides nucleic acid primers and amplified probes for detecting allelic imbalance at this region of the human genome for the detection of breast cancer (e.g. SEQ ID Nos: 24 and 25) which amplify an intron region of the gene encoding an EDD protein. As with the CEDD microsatellite, the nucleic acid that is amplified using the primers set forth in SEQ ID Nos: 24 and 25 is also useful as a hybridization probe and the present invention clearly encompasses such a use.

In one embodiment, the amplification reaction detection means described supra is further coupled to a classical hybridization reaction detection means to further enhance sensitivity and specificity of the inventive method, such as by hybridizing the amplified DNA with a probe which is different from any of the probes used in the amplification reaction.

In another embodiment, the hybridization reaction detection means described supra is further coupled to a second hybridization step employing a probe which is different from the probe used in the first hybridization reaction.

The comparison to be performed in accordance with the present invention may be a visual comparison of the signal generated by the probe, or alternatively, a comparison of data integrated from the signal, such as, for example, data that have been corrected or normalized to allow for variation between samples. Such comparisons can be readily performed by those skilled in the art.

In one embodiment, the method supra further comprises isolating the test sample and/or the reference sample from one or more suitable subjects (i.e. the individual being tested and/or one or more other subjects who are suitable to provide a reference sample). In this respect it is within the scope of the invention for the reference sample and the test sample to be isolated from the same subject at different time points, or alternatively, from different tissues of the same subject. Preferably, the test sample and the reference sample are from the same tissue type, or a tissue comprising cells of the same type.

In one embodiment, the test sample and/or the reference sample(s) has/have been obtained previously from the subject(s).

In another embodiment, the present invention provides a method for diagnosing a cancer or predicting recurrence of a cancer in a subject comprising determining the level of mRNA or protein encoded by nucleic acid linked to map position 8q22.3 of the human genome in a sample of said subject, wherein an elevated level of said mRNA or protein is indicative of relapse of a cancer in said subject.

In one embodiment, the mRNA encoded by a nucleic acid linked to map position 8q22.3 of the human genome encodes an EDD protein. Preferably, the mRNA comprises the sequence set forth in SEQ ID NO: 1 or SEQ ID NO: 3 or a fragment thereof. Even more preferably, the mRNA encodes a protein comprising the sequence set forth in SEQ ID NO: 2 or SEQ ID NO: 4 or a fragment thereof.

In one embodiment the protein encoded by a nucleic acid linked to map position 8q22.3 of the human genome is an EDD protein. Preferably the protein encoded by a nucleic acid linked to map position 8q22.3 of the human genome comprises the sequence set forth in SEQ ID NO: 2 or SEQ ID NO: 4 or a fragment thereof.

Methods of determining the amount of mRNA or protein encoded by a nucleic acid linked to map position 8q22.3 are well known in the art and/or described herein.

As exemplified herein, the level of expression of EDD mRNA and EDD protein is predictive of recurrence of ovarian cancer in subjects that have received treatment for said cancer. As used herein the term “recurrence” or “relapse” shall be understood to mean that, following treatment for a cancer, a subject has developed a further cancer. The cancer that has redeveloped may be the same form of cancer for which the patient received treatment or a different form of cancer, for example in the case of a cancer that has metastasized.

In an alternative embodiment the invention provides a method for detecting allelic imbalance in a region of the human genome comprising hybridizing a nucleic acid probe or primer to genomic DNA and detecting the hybridization, wherein the probe or primer comprises a nucleotide sequence selected from the group consisting of:
(i) the sequence set forth in SEQ ID NO: 5;
(ii) the sequence set forth in SEQ ID NO: 6;
(iii) the sequence set forth in SEQ ID NO: 7;
(iv) the sequence set forth in SEQ ID NO: 24;
(v) the sequence set forth in SEQ ID NO: 25; and
(vi) the sequence of a nucleic acid fragment produced by amplification using any one of (i) to (v) as amplification primers in PCR.

The embodiments described supra for nucleic acid detection are to be applied mutatis mutandis to this embodiment of the invention. Preferably, the hybridization is detected by amplifying nucleic acid using said probe or primer in a PCR reaction, or alternatively, by labelling the probe with a suitable reporter molecule and detecting the signal generated by the reporter molecule.

In another embodiment, the present invention provides a method for detecting a cancer cell in a subject, said method comprising:
(i) determining the level of mRNA encoded by nucleic acid linked to map position 8q22.3 of the human genome that is expressed in a test sample from said subject; and
(ii) comparing the level of the mRNA determined at (i) to the level of mRNA encoded by nucleic acid linked to map position 8q22.3 of the human genome that is expressed in a reference sample from a healthy or normal individual,
   wherein a level of the mRNA at (i) that is enhanced in the test sample relative to the reference sample from the normal or healthy individual is indicative of the presence of a cancer cell in said subject.

In one embodiment the mRNA encodes an EDD-protein. In an alternative embodiment, the mRNA encodes a p53R2 protein.

By “mRNA encoding an EDD protein” is meant mRNA encoding a EDD polypeptide that has at least about 80% identity to SEQ ID NO: 2 or 4, and, more particularly, mRNA comprising a nucleotide sequence that has at least about 80% identity, more preferably at least about 95% identity, and still more preferably at least about 99% identity to the nucleotide sequence set forth in SEQ ID NO: 1 or 3.

The tissue samples, general hybridization and amplification methods, and detection and analysis methods described supra for detecting allelic imbalance in this region of the human genome are applied mutatis mutandis to the detection of mRNA without any undue experimentation. Alternatively, or in addition, the level of mRNA encoding an EDD protein or p53R2 protein in a patient sample is analyzed by a variations on these procedures that are well known in the art such as, for example, in situ hybridization to mRNA, northern blotting techniques, or RT-PCR analysis (such as, for example, performed on laser capture microdissected samples). Microarray technology is readily applied to such embodiments for high throughput screening of samples.

In hybridization-based approaches, the use of riboprobes is particularly preferred because RNA/RNA duplexes are more stable than RNA/DNA duplexes. As with DNA/DNA hybridizations, the conditions for RNA/DNA or RNA/RNA hybridization and/or wash will vary depending upon the nature of the hybridization matrix used to support the sample RNA, or the type of hybridization probe used.

For RT-PCR, or a variant thereof, one or more nucleic acid probes molecules of at least about 20 contiguous nucleotides in length is hybridized to cDNA or cRNA that has been reverse-transcribed from the target mRNA, and nucleic acid copies of the template are enzymically-amplified using nucleic acid primers. RT-PCR is particularly useful when it is desirable to determine gene expression levels. It is also known to those skilled in the art to use mRNA/DNA hybrid molecules as a template for such amplification reactions, and, as a consequence, first strand cDNA synthesis is all that is required to be performed prior to the amplification reaction. Variations of the embodiments described herein are described in detail by McPherson et al., PCR: A Practical Approach. (series eds, D. Rickwood and B. D. Hames), IRL Press Limited, Oxford. pp 1-253, 1991.

In one embodiment, the method supra further comprises isolating the test sample and/or the reference sample from one or more suitable subjects (i.e. the individual being tested and/or one or more other subjects who are suitable to provide a reference sample). In this respect it is within the scope of the invention for the reference sample and the test sample to be isolated from the same subject at different time points, or alternatively, from different tissues of the same subject. Preferably, the test sample and the reference sample are from the same tissue type, or a tissue comprising cells of the same type.

In one embodiment, the test sample and/or the reference sample(s) has/have been obtained previously from the subject(s).

As exemplified herein, probes and primers having the nucleotide sequences set forth in any one of SEQ ID Nos: 26 to 30, 33 or 34, 37, 38, or 40 are used to detect breast cancer cells. As with microsatellite detection, the nucleic acid primers set forth in any one of SEQ ID Nos: 26 to 30, 33 or 34, 37, 38, or 40 can also be used as a probe to detect nucleic acid directly. As with microsatellite detection, nucleic acid that has been amplified with any one of SEQ ID Nos: 26 to 30, 33 or 34, 37, 38, or 40 is also useful as a hybridization probe and the present invention clearly encompasses such a use.

A further embodiment of the present invention relates to nucleic acid probes for detecting a cancer in accordance with the embodiments described supra.

In one embodiment, the present invention provides an isolated nucleic acid molecule for detecting a cancer cell comprising a nucleotide sequence selected from the group consisting of:
(i) a sequence that encodes the amino acid sequence set forth in SEQ ID NO: 4 wherein said amino acid sequence lacks the sequence VLLLPL;
(ii) the sequence set forth in SEQ ID NO: 3;
(iii) the sequence set forth in SEQ ID NO: 5;
(iv) the sequence set forth in SEQ ID NO: 6;
(v) the sequence set forth in SEQ ID NO: 7;
(vi) the sequence set forth in SEQ ID NO: 24;
(vii) the sequence set forth in SEQ ID NO: 25;
(viii) the sequence of a nucleic acid fragment produced by amplification using (vi) and (vii) as amplification primers in PCR;
(ix) the sequence set forth in SEQ ID NO:26;
(x) the sequence set forth in SEQ ID NO: 27;
(xi) the sequence of a nucleic acid fragment produced by amplification using (ix) and (x) as amplification primers in PCR;
(xii) the sequence set forth in SEQ ID NO: 28;
(xii) the sequence set forth in SEQ ID NO: 29;
(xiii) the sequence set forth in SEQ ID NO: 30;
(xiv) the sequence of a nucleic acid fragment produced by amplification using (xii) and (xiii) as amplification primers in PCR;
(xv) the sequence set forth in SEQ ID NO: 33;
(xvi) the sequence set forth in SEQ ID NO: 34;
(xvii) the sequence of a nucleic acid fragment produced by amplification using (xv) and (xvi) as amplification primers in PCR;
(xviii) the sequence set forth in SEQ ID NO: 37;
(xix) the sequence set forth in SEQ ID NO: 38;
(xx) the sequence of a nucleic acid fragment produced by amplification using (xviii) and (xix) as amplification primers in PCR;
(xxi) the sequence set forth in SEQ ID NO: 40; and
(xxii) a sequence that is complementary to any one of (i) to (xxi).

As used herein, the term “nucleic acid” shall be taken to mean any single-stranded or double-stranded RNA, DNA, cDNA, cRNA, or synthetic oligonucleotide, or alternatively, an analog of RNA, DNA, cDNA, CRNA, or a synthetic oligonucleotide. “Nucleic acid” also includes any genomic gene equivalents of a cDNA molecule.

The isolated nucleic acid of the invention will hybridize to nucleic acid from a human or a non-human mammal.

Protein Complexes Comprising an EDD Protein and Diagnostic Uses Therefor

The present inventors have also identified several expression products of a gene linked to map position 8q22.3 of the human genome wherein each of said expression products consists of a protein-protein interaction involving the EDD protein with a protein selected from the group consisting of a protein having tumor suppressor activity or cell cycle modulatory activity or DNA repair activity, a progesterone receptor protein, a nuclear targeting protein and a calcium/integrin binding protein (CIB). The interactions provide novel protein-protein complexes that are useful for detecting DNA damage, progesterone-mediated effects in cells, such as for example, progestin-sensitive tumorigenesis or tumor growth, ubiquitin-mediated proteolysis in cells, or changes in vascularization in a subject. The novel protein-protein complexes are also useful for producing diagnostic reagents such as, for example, antibodies. As will be known to those skilled in the art, antibodies are also useful for therapeutic applications. Additionally, the novel protein-protein complexes form the bases of assays for identifying modulatory compounds, including small molecule agonists or antagonists of the protein-protein interactions, such as for the treatment of hyperproliferative disorders, preventing cell proliferation or enhancing repair to damaged DNA, or for targeting cells having damaged DNA and/or that are tumor cells.

Accordingly, one embodiment of the present invention provides an isolated or recombinant protein complex comprising:
(i) an EDD protein or a portion of an EDD protein sufficient to bind to a protein selected from the group consisting of a protein having tumor suppressor activity, a protein having cell cycle modulatory activity, a protein associated with DNA repair or damage, a nuclear targeting protein, a progesterone receptor protein and a protein associated with vascularization; and
(ii) a nuclear protein selected from the group consisting of a protein having tumor suppressor activity, a protein having cell cycle modulatory activity, a protein associated with DNA repair or damage, a nuclear targeting protein, a progesterone receptor protein and a protein associated with vascularization or a portion of said protein sufficient to bind to said EDD protein or said portion of an EDD protein.

As used herein, the term “protein having tumor suppressor activity” shall be taken to mean any protein or polypeptide that is known or thought to be involved repressing or reducing or preventing tumorigenesis or tumor growth, such as, for example, by activating a cellular response to DNA damage, assisting DNA repair, restoring survival after DNA damage (e.g. by interacting with and phosphorylating BRCA1 or BRCA2, thereby allowing BRCA1 or BRCA2 to restore survival after DNA damage), or preventing cellular proliferation of tumor cells (e.g. by stabilizing a tumor suppressor protein such as p53, thereby leading to cell cycle arrest in G1). Such a functional assignment is readily determined in the art by examining the effects of mutations in genes encoding the protein having tumor suppressor activity, or alternatively, by empirical data showing a direct effect on tumorigenesis or tumor growth. Preferably, the protein having tumor suppressor activity will be a nuclear protein. Exemplary proteins having tumor suppressor activity in the present context include a member of the CDS1 subfamily of serine/threonine protein kinases, p53 (TP53), TNF-alpha, HSP70, estrogen receptor, androgen receptor, progesterone receptor, HRAS1-VNTR, CHK2, BRCA1, BRCA2, AIB1, NAT1, NAT2, XRCC1, XRCC2, XRCC5, CIB, importin alpha-1, importin alpha-3, and importin alpha-5.

In one preferred embodiment, the protein having tumor suppressor activity will comprise an amino acid sequence having at least about 80% identity to a sequence set forth in any one of SEQ ID NOs: 9, 11, 13, 15, 17, 19, 21, or 23.

For the purposes of nomenclature, the amino acid sequence set forth in SEQ ID NO: 9 consists of a human importin alpha-1 protein (i.e. karyopherin alpha-2) which is encoded by nucleotides 133 to 1722 of the nucleotide sequence set forth in SEQ ID NO: 8. The amino acid sequence set forth in SEQ ID NO: 11 consists of a human importin alpha-3 protein (i.e. karyopherin alpha-4) which is encoded by nucleotides 10 to 1575 of the nucleotide sequence set forth in SEQ ID NO: 10. The amino acid sequence set forth in SEQ ID NO: 13 consists of a human importin alpha-5 protein (i.e. karyopherin alpha-1) which is encoded by nucleotides 47 to 1663 of the nucleotide sequence set forth in SEQ ID NO: 12. The amino acid sequence set forth in SEQ ID NO: 15 consists of a human progesterone receptor protein (PR) which is encoded by nucleotides 176 to 2977 of the nucleotide sequence set forth in SEQ ID NO: 14. The amino acid sequence set forth in SEQ ID NO: 17 consists of a human CIB/KIP protein which is encoded by nucleotides 67 to 642 of the nucleotide sequence set forth in SEQ ID NO: 16. The amino acid sequence set forth in SEQ ID NO: 19 consists of a human CHK2 protein (i.e. transcript variant 1) which is encoded by nucleotides 762 to 2393 of the nucleotide sequence set forth in SEQ ID NO: 18. The amino acid sequence set forth in SEQ ID NO: 21 consists of a human CHK2 protein (i.e. transcript variant 2) which is encoded by nucleotides 762 to 2306 of the nucleotide sequence set forth in SEQ ID NO: 20. The amino acid sequence set forth in SEQ ID NO: 23 consists of a human BRCA2 protein which is encoded by nucleotides 229 to 10,485 of the nucleotide sequence set forth in SEQ ID NO: 22.

More preferably, a protein having tumor suppressor activity will comprise an amino acid sequence having at least about 80% identity to a sequence set forth in any one of SEQ ID NOs: 19, 21, or 23 (i.e. at least 80% identity to a human CHK2 protein or a human BRCA2 protein) or a portion thereof sufficient to bind an EDD protein.

Those skilled in the art will be aware that CHK2 is a nuclear cell cycle checkpoint regulatory protein of the CDS1 subfamily of serine/threonine protein kinases, having DNA repair function. CHK2 contains a forkhead-associated protein interaction domain essential for activation in response to DNA damage and is rapidly phosphorylated in response to replication blocks and DNA damage. When activated, the encoded protein is known to inhibit CDC25C phosphatase, preventing entry into mitosis. CHK2 also interacts with BRCA1 protein, causing BRCA1 to be phosphorylated, thereby allowing BRCA1 to restore survival after DNA damage. CHK2 also has a putative tumor suppressor activity by virtue of stabilizing the tumor suppressor protein p53 (TP53), leading to cell cycle arrest in the G1 phase.

Those skilled in the art will also be aware that BRCA2 is a tumor suppressor protein by virtue of mutations, typically microdeletions, in the BRCA2-encoding gene being linked to an elevated risk of young onset breast cancer. BRCA2 is also associated with the activation of double-strand break repair and/or homologous recombination. The similar properties of BRCA1 and BRCA2, for example their co-localization in a biochemical complex, and similar function, suggests that these proteins function in the same genetic pathway. The amino acid sequences of BRCA1 and BRCA2 comprise transcriptional activation protein domains.

By “protein having cell cycle modulatory activity” is meant that the protein functions either solus or in cooperation with one or more other proteins or nucleic acid, to enhance cell cycle progression or to inhibit progression from one stage of the cell cycle to another. Preferred cell cycle modulatory proteins will modulate the progression of cells from the G1 into the G2 phase, or the entry of cells into mitosis. Such a functional assignment is readily determined in the art by examining the effects of mutations in genes encoding the protein having cell cycle modulatory activity, or alternatively, by empirical data showing a direct effect on cell cycle progression, or by analysing protein-protein interactions with known cell cycle modulatory proteins. Preferably, the protein having cell cycle modulatory activity will be a nuclear protein. Exemplary proteins having cell cycle modulatory activity in the present context include a member of the CDS1 subfamily of serine/threonine protein kinases, Cdc25, CDC2a, cyclin-dependent kinase (CDK), CDK inhibitor, a mitogenic cyclin (e.g., cyclin A, cyclin B, cyclin D, etc), p53 (TP53), and CHK2.

In one preferred embodiment, the protein having tumor suppressor activity will comprise an amino acid sequence having at least about 80% identity to a sequence set forth in any one of SEQ ID NOs: 19 or 21.

Those skilled in the art will be aware from the preceding discussion that the Cdc25, CHK2 and TP53 proteins act as cell cycle modulatory proteins in the present context. In a particularly preferred embodiment, the protein having cell cycle modulatory activity is CHK2 (SEQ ID Nos: 19 or 21) or a portion thereof sufficient to bind an EDD protein.

By “protein associated with DNA repair or damage” is meant a nuclear protein that functions in modulating DNA repair, is activated by DNA damage or otherwise activates double-strand break repair, DNA mismatch repair, homologous recombination or DNA end-ligation events in a cell or in vitro. Such a functional assignment is readily determined in the art by examining the effects of mutations in genes encoding the protein, or alternatively, by empirical data showing a direct effect on the ability of a cell to respond to DNA damage and/or to undergo DNA repair and/or to activate homologous recombination or DNA end-ligation.

Exemplary proteins associated with DNA repair or damage in the present context include a member of the CDS1 subfamily of serine/threonine protein kinases, BRCA1, BRCA2, CIB/KIP, TP53, MLH1, MSH2, ATM, CHK2, XRCC1, XRCC2,

XRCC5 and importin alpha-5.

In one preferred embodiment, the protein associated with DNA damage or repair will comprise an amino acid sequence having at least about 80% identity to a sequence set forth in any one of SEQ ID NOs: 13, 17, 19, 21, or 23.

In a particularly preferred embodiment, the protein associated with DNA damage or repair is selected from the group consisting of a CHK2 protein (SEQ ID Nos: 19 or 21), a BRCA2 protein (SEQ ID NO: 23), a CIB protein (SEQ ID NO: 17) and an importin alpha-5 protein (SEQ ID NO: 13) or a portion thereof sufficient to bind an EDD protein.

Those skilled in the art will be aware from the preceding discussion that the BRCA2 and CHK2 proteins are associated with DNA damage or repair, primary acting as DNA-damage checkpoint control proteins. Those skilled in the art will also be aware that the CIB protein is a member of the calcium-binding protein family that interacts with a DNA-dependent protein kinase and, as a consequence, is in the art to play a role in kinase-phosphatase regulation of DNA end-ligation events. The CIB protein also interacts with integrin alpha(IIb)beta(3), which may implicate this protein as a regulatory molecule for integrin alpha(IIb)beta(3). Those skilled in the art will also be aware that the importin alpha-5 protein is involved in ds-DNA break repair. Importin alpha-5 is also recruited by RAG1 during V(D)J recombination, the process by which genes encoding immunoglobulins and T-cell receptors are generated.

By “nuclear targeting protein ” is meant a chaperonin protein that assists with the translocation of a protein from the cytosol to the nucleus. The import of proteins into the nucleus involves an energy-independent docking of the protein to the nuclear envelope and an energy-dependent translocation through the nuclear pore complex. In the present context, a nuclear targeting protein will facilitate one or both of these processes. Such a functional assignment is readily determined in the art by examining the effects of mutations in genes encoding the protein, or alternatively, by empirical data showing a direct effect on the ability of a protein to facilitate nuclear localisation of a protein. Exemplary nuclear targeting proteins include importin alpha-1 (SEQ ID NO: 9), importin alpha-3 (SEQ ID NO: 11) and importin alpha-5 (SEQ ID NO: 13).

In one preferred embodiment, the nuclear targeting protein will comprise an amino acid sequence having at least about 80% identity to a sequence set forth in any one of SEQ ID NOs: 9, 11 or 13.

Those skilled in the art are aware that importin alpha-1 and importin alpha-3: interact with the nuclear localisation sequence (NLS) of the DNA helicase Q1 protein and the NLS of the SV40 T antigen, and dock the proteins to the nuclear envelope or nuclear pore complex in the nuclear transport of those proteins. These importins are also considered in the art to play a role in V(D)J recombination.

By “nuclear localisation sequence” or “NLS” is meant a short region of basic amino acids or 2 such regions spaced about 10 amino acids apart that is required for nuclear localization of a protein, particularly transport mediated by an importin protein.

The functional meaning of the term “progesterone receptor protein” will be apparent from the preceding description. For the purposes of defining the structure of a progesterone receptor protein, there is provided the amino acid sequence set forth herein as SEQ ID NO: 15. Variants of SEQ ID NO: 15, such as, for example, proteins having at least about 80% identity thereto are also within the scope of the present invention, the only requirement being that the variant is a functional progesterone receptor or derived from a functional progesterone receptor protein. A human progesterone receptor protein has been shown by the present inventors to bind to an EDD protein.

By “a protein associated with vascularization” is meant that the protein functions to enhance the development of new blood vessels in a subject either directly or indirectly (eg by inducing expression of a protein that enhances vascularization or interacting with a protein that enhances or inhibits vascularization). Preferably, the protein is associated with vasculogenic activity, whereby the term “vasculogenic activity” shall be understood to mean the formation of new blood vessels de novo. The process of vasculogenesis results in the in situ differentiation of mesodermal progenitor cells to endothelial cells that organize into a primitive vascular network. Many aggressive tumors (eg melanoma tumor cells) are also capable of inducing vasculogenesis in a subject.

In another embodiment, a protein associated with vascularization is capable of enhancing or inhibiting angiogenesis. By “angiogenesis” is meant the development of new blood vessels from previously existing blood vessels. The process of angiogenesis involves the endothelial cells of a pre-existing blood vessel secreting membranes to erode the basement membrane of the vessel. Endothelial cells subsequently proliferate and migrate toward an angiogenic signal, forming tight cell junctions with other endothelial cells in order to form a new blood vessel. Angiogenesis occurs during, for example, embryonic development, wound healing, and formation of the corpus luteum, endometrium and placenta. However, aberrant angiogenesis is associated with a number of disorders, including, tumor metastasis.

A functional assignment of a protein that is associated with vascularization is readily determined in the art by examining the effects of mutations in genes encoding the protein having vascularization activity, or alternatively, by empirical data showing a direct effect on vascularisation, or by analysing protein-protein interactions with known vascularization modulatory proteins. For example, the effect of a protein on vascularization may be determined any assay known in the art, such as, for example, a bovine capillary endothelial cell proliferation assay, a chick CAM assay (as described in O'Reilly et al, Cell, 79(2): 315-328 1994), a mouse comeal assay or a mouse ischemic retinopathy assay (as described in Ozaki et al, Am. J. Path., 156(2): 697-707, 2000).

Preferably, the protein having vascularization activity will be a nuclear protein. For example, proteins with vascularization activity include, transforming growth factor β, lipid phosphatase LPP3, c-Myc, dimethylarganine dimethylaminohydrolase (DDAH), cytochrome P450, Tie-2 and VE-cadherin.

In one embodiment, the protein complex is a heterodimer or hetero-multimeric protein comprising an EDD protein. Those skilled in the art will be aware that a heterodimer or heterodimeric protein complex comprises two different peptide or polypeptide subunits. As used herein, the term “heteromultimer” shall be taken to mean a higher order protein complex comprising at least three peptide or polypeptide subunits, wherein at least two of said subunits are different. For example, a heterohexameric protein is known to comprise six peptide or polypeptide subunits, and, in the present context, may comprise three different homodimers, or six different monomers, or a dimer and a tetramer of different protein, etc. Accordingly, the present invention is not to be limited by the composition and size of the protein complex, the only requirement being that at least one peptide or polypeptide subunit consists of EDD or a portion of EDD, and at least one other peptide or polypeptide subunit consists of a polypeptide selected from the group consisting of a protein having tumor suppressor activity, a protein associated with DNA damage or repair, a cell cycle modulatory protein, a nuclear targeting protein and a progesterone receptor protein, a portion of a protein having tumor suppressor activity, a portion of a protein associated with DNA damage or repair, a portion of a cell cycle modulatory protein, a portion of a nuclear targeting protein and a portion of a progesterone receptor protein.

The protein subunits of the protein complex are held in physical relation by any means known to those skilled in the art. This physical relation may involve the formation of an induced magnetic field or paramagnetic field, covalent bond formation such as a disulfide bridge formation between polypeptide subunits, an ionic interaction such as occur in an ionic lattice, a hydrogen bond or alternatively, a van der Waals interaction such as a dipole-dipole interaction, dipole-induced-dipole interaction, induced-dipole-induced-dipole interaction or a repulsive interaction or any combination of the above forces of attraction. Alternatively, the peptide or polypeptide subunits may be held in physical relation by expressing them as a fusion polypeptide, optionally separated by a spacer to permit their folding. Accordingly, the physical relation between the peptide or polypeptide subunits may be a consequence of their binding capability and attraction toward one another, or alternatively, a consequence of their mode of production.

Preferably, the peptide, polypeptide or protein partners are in direct physical relation. By “direct physical relation” is meant that the binding partners contact each other without any intervening protein moiety or non-protein moiety. However, the protein complexes of the present invention can clearly include one or more additional protein moieties or non-protein moieties, such as, for example, a protein or non-protein moiety that enhances or stabilizes the physical relation between EDD or a portion of EDD and the other binding partner.

The present invention further encompasses a protein complex wherein one or more of the binding partners include a post-translational modification, such as, for example, a phosphorylated, fucosylated, myristoylated, farnesylated, or glycosylated residue. Such post-translational modifications may enhance complex formation or stabilize the complex once it is formed. Phosphorylation of one or more serine or tyrosine residues present on one or more of the binding partners including EDD or CHK2, such as by a member of the CDS1 subfamily of serine/threonine protein kinases (e.g. CHK2), is also contemplated. Ubiquitination of one or more binding partners is also not to be excluded.

Preferably, the binding partners of the protein complex are mammalian polypeptides or proteins, and more preferably of human origin. It is not strictly necessary for the binding partners to be derived from the same source, however this is preferred because the ability of the partners to associate or be maintained in physical non-covalent association with each other is generally enhanced if they are derived from the same organism.

Those skilled in the art will be in a position to determine a suitable portion of EDD or other polypeptide that can form the protein complex of the invention, such as, for example a portion comprising a cysteine/histidine rich region (e.g. a zinc-binding domain), HECT domain, RING domain, nuclear localisation sequence (NLS), UBA domain that binds to mono- or multi-ubiquitin chains, or a region comprising alpha helices (e.g. carboxy-terminal 60 amino acids of SEQ ID Nos: 2 or 4). This is achieved, for example, using conventional binding assays for determining the binding between two proteins without undue experimentation.

Similarly, the skilled artisan can readily determine a portion of the other binding partner that is sufficient to bind to an EDD protein or a portion thereof. Again, conventional binding assays for determining the binding between two proteins may be used to assay the suitability of such portions.

Preferably, a portion of an EDD protein or other protein suitable for protein complex formation comprises at least about 5 amino acids in length, more preferably at least about 10 amino acids in length, even more preferably at least about 15 amino acids in length and still more preferably at least about 20 or 30 or 40 or 50 amino acids in length.

In a preferred embodiment, the portion of an EDD protein that is sufficient to bind to a protein having tumor suppressor activity or having cell cycle modulatory activity will at least comprise amino acid residues from about position 1400 to about position 2550 of SEQ ID NO: 2 or the corresponding region in SEQ ID NO: 4, or more preferably, the region of SEQ ID NO: 2 from about position 1406 to about position 2526.

In another embodiment, the portion of an EDD protein that is sufficient to bind to a protein associated with DNA repair or damage will at least comprise amino acid residues in the C-terminal portion of the full-length EDD protein, such as, for example, from about position 889 to about position 2799 of SEQ ID NO: 2 or the corresponding region in SEQ ID NO: 4.

In another embodiment, the portion of an EDD protein that is sufficient to bind to a nuclear targeting protein will at least comprise the nuclear localisation sequence of an EDD protein. In one embodiment, the EDD NLS comprises at least amino acid residues from about position 502 to about position 517 of SEQ ID NO: 2, or from about position 600 to about position 605 of SEQ ID NO: 2, or from about position 1222 to about position 1241 of SEQ ID NO: 2, or the corresponding regions in SEQ ID NO: 4.

In another embodiment, the portion of an EDD protein that is sufficient to bind to a progesterone receptor protein will at least comprise the N-terminal portion of EDD, such as, for example, amino acid residues from about position 1 to about position 889 of SEQ ID NO: 2, or the corresponding regions in SEQ ID NO: 4, or more preferably, the region of SEQ ID NO: 2 from about position 420 to about position 889 or the equivalent region in SEQ ID NO: 4.

A preferred portion of the CHK2 protein that interacts with an EDD protein will at least comprise the FHA domain of CHK2 (i.e. amino acid residues 117 to 157 of SEQ ID NO: 19 or the equivalent region in SEQ ID NO: 21, and more preferably residues 111 to 177 of SEQ ID NO: 19 or the equivalent region in SEQ ID NO: 21).

A preferred portion of a progesterone receptor (PR) protein that interacts with an EDD protein will comprise at least a C-terminal portion of PR, more preferably a region comprising a the hinge domain, DNA binding domain and ligand-dependent activation domain-2 of the full-length receptor protein (i.e. “CDE region”).

A preferred portion of an importin protein that interacts with an EDD protein will at least comprise the C-terminal domain of importin, such as, for example, amino acids from about position 229 to about position 538 of SEQ ID NO: 13 or the homologous region in SEQ ID NO: 9.

Another embodiment of the present invention provides isolated peptides, polypeptides, and kits comprising same for producing the protein complex, or for identifying a modulator of a biological interaction between EDD or a portion of EDD and one or more other polypeptides selected from the group consisting of a protein having tumor suppressor activity, a protein having cell cycle modulatory activity, a protein associated with DNA repair or damage, a nuclear targeting protein, a progesterone receptor protein, and a protein associated with vascularization, or a portion thereof.

In one embodiment, the kit comprises a first polypeptide consisting of EDD or a portion thereof sufficient to bind to a protein selected from the group consisting of a protein having tumor suppressor activity, a protein having cell cycle modulatory activity, a protein associated with DNA repair or damage, a nuclear targeting protein, a progesterone receptor protein and a protein associated with vascularization, and a second polypeptide consisting of a protein selected from the group consisting of a protein having tumor suppressor activity, a protein having cell cycle modulatory activity, a protein associated with DNA repair or damage, a nuclear targeting protein, a progesterone receptor protein and a protein associated with vascularization or a portion thereof sufficient to bind to said EDD protein or said portion of an EDD protein. Such kits are used to produce protein complexes comprising: EDD.

Optionally, the kit further includes a protein or a portion thereof sufficient to bind to a protein that binds EDD comprising an amino acid sequence selected from the group consisting of SEQ ID NO: 9, SEQ ID NO: 11, SEQ ID NO: 13, SEQ ID NO: 15, SEQ ID NO: 17, SEQ ID NO: 19, SEQ ID NO: 21, and SEQ ID NO: 23. In accordance with this embodiment, the subject kit is used to produce higher order protein complexes comprising an EDD protein and a protein that binds EDD and a protein that binds to the EDD-binding protein. Alternatively, such kits are useful for analyzing competition between EDD and a protein that binds to an EDD-binding protein in the formation of a protein-protein complex. For example, the kit can be used to produce a complex comprising EDD and BRCA1 and CHK2 or to determine competition between BRCA1 and EDD for complex formation with CHK2. Similarly, the kit can be used to produce a complex comprising EDD and BRCA2 and CHK2 or to determine competition between BRCA2 and EDD for complex formation with CHK2, or between CHK2 and EDD for complex formation with BRCA2. Similarly, the kit can be used to produce a complex comprising EDD and BRCA2 and BRCA1 or to determine competition between BRCA1 and EDD for complex formation with BRCA2. Similarly, the kit can be used to produce a complex comprising EDD and TP53 and CHK2 or to determine competition between TP53 and EDD for complex formation with CHK2. Similarly, the kit can be used to produce a complex comprising EDD and CIB and integrin or to determine competition between integrin and EDD for complex formation with CIB. Similarly, the kit can be used to produce a complex comprising EDD and importin alpha-5 and RAG1 or to determine competition between RAG1 and EDD for complex formation with importin alpha-5. Similarly, the kit can be used to produce a complex comprising EDD and importin alpha-1/3 and DNA helicase or to determine competition between DNA helicase and EDD for complex formation with importin alpha-1/3. Similarly, the kit can be used to produce a complex comprising EDD and progesterone receptor and a heat shock protein that binds to the progesterone receptor or to determine competition between a heat shock protein and EDD for complex formation with the progesterone receptor.

The kit may also include one or more antibodies or ligands that bind to the first polypeptide or the second polypeptide, or to any one or more of the protein complexes supra comprising EDD, such as, for example, an antibody or ligand that specifically recognizes an assembled protein complex or the conformation of said protein complex, rather than the individual polypeptide components per se.

In another embodiment, the kit comprises:
(a) a first compartment comprising an EDD protein or a portion thereof sufficient to form a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor; and
(b) a second compartment comprising an antibody or ligand that binds to a protein selected from the group consisting of (i) a CHK2 protein; (ii) a BRCA2 protein; (iii) a CIB protein; (iv) an importin alpha-1 protein; (v) an importin alpha-3 protein; (vi) an importin alpha-5 protein; and (vii) a progesterone receptor protein, or an antibody or ligand that binds to a protein complex selected from the group consisting of: (i) a complex comprising EDD and CHK2; (ii) a complex comprising EDD and BRCA2;

(iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor.

wherein said antibody or ligand that binds to a protein complex does not bind to the individual protein binding partners.

In another embodiment, the kit comprises a first compartment comprising an antibody or ligand that binds to an EDD protein and a second compartment comprising a protein selected from the group consisting of (i) a CHK2 protein; (ii) a BRCA2 protein; (iii) a CIB protein; (iv) an importin alpha-1 protein; (v) an importin alpha-3 protein; (vi) an importin alpha-5 protein; and (vii) a progesterone receptor protein, or a portion thereof sufficient to bind to an EDD protein.

In another embodiment, the kit comprises:
(a) a first compartment comprising an isolated or recombinant protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor; and
(b) a second compartment comprising an (i) antibody or ligand that binds to a polypeptide selected from the group consisting of a CHK2 protein, a BRCA2 protein, a CIB protein, an importin alpha-1 protein, an importin alpha-3 protein, an importin alpha-5 protein, a progesterone receptor protein and an EDD protein; or (ii) an antibody or ligand that binds to one or more protein complexes (a).

As used herein, the term “antibody” refers to intact monoclonal or polyclonal antibodies, immunoglobulin (IgG, IgM, IgE) fractions, humanized antibodies, or recombinant single chain antibodies, as well as fragments thereof, such as Fab, F(ab′)₂, and Fv, which are capable of binding a linear or conformational epitope of at least one binding partner of the protein complex, or to a conformational epitope of the assembled protein complex. Humanized antibodies are antibodies in which amino acids have been replaced in the non-antigen binding regions in order to more closely resemble a human antibody, while still retaining the original binding ability.

In one embodiment, antibodies are obtained from a commercial source, such as for example, Santa Cruz Biotechnology, Inc, CA 95060, USA. Other commercial sources will be well known to those skilled in the art.

Alternatively, antibodies are produced by conventional means. For the production of antibodies, an intact polypeptide, or a portion thereof containing a short amino acid sequence of interest is used as the immunizing antigen or immunogen. The immunogen is derived from a natural source, produced by recombinant expression means or by in vitro translation of RNA, or synthesized chemically such as by Fmoc chemistry: Immunogens consisting of short peptides a preferably conjugated to a carrier protein, such as, for example bovine serum albumin (BSA), thyroglobulin, or keyhole limpet hemocyanin (KLH), prior to immunization. The coupled peptide is then used to immunize the animal. Various host animals (e.g. goats, rabbits, rats, mice, dogs, humans) are immunized by intramuscular, intraperitoneal, or intravenous injection, with immunogen, optionally in the presence of an adjuvant to enhance the immune response to the immunogen. Preferred adjuvants include, for example, Freund's complete or incomplete adjuvant, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are preferred.

Monoclonal antibodies are prepared using any technique which provides for the production of antibody molecules by continuous cell lines in culture, such as, for example, the hybridoma technique, the human B-cell hybridoma technique, and the EBV-hybridoma technique (Kohler et al. Nature 256, 495-497, 1975; Kozbor et al., J. Immunol. Methods 81, 31-42, 1985; Cote et al., Proc. Natl. Acad. Sci. USA 80, 2026-2030, 1983; Cole et al., Mol. Cell Biol. 62, 109-120, 1984).

Techniques developed for the production of chimeric antibodies are also employed. Such techniques involve splicing a mouse antibody gene to a human antibody gene to produce a molecule having the desired antigen specificity and biological activity (Morrison et al., Proc. Natl. Acad. Sci. USA 81, 6851-6855, 1984; Neuberger et al., Nature 312, 604-608, 1984; Takeda et al., Nature 314, 452-454, 1985).

Alternatively, techniques described for the production of single chain antibodies are adapted, using methods known in the art, to produce single chain antibodies having the desired specificity.

Antibodies are also produced by inducing in vivo production in the lymphocyte population or by screening immunoglobulin libraries or panels of highly specific binding reagents as disclosed by Orlandi et al., Proc. Natl. Acad. Sci. USA 86, 3833-3837, 1989; Winter et al., Nature 349, 293-299, 1991).

Antibody fragments, such as, for example, F(ab′)₂ fragments, are produced by pepsin digestion of an intact antibody molecule. Fab fragments are generated by reducing the disulfide bridges of F(ab′)₂ fragments. Alternatively, Fab expression libraries are constructed, to allow rapid and easy identification of monoclonal Fab fragments with the desired specificity (Huse et al., Science 254, 1275-1281, 1989).

The antibodies or ligands may assist in the subsequent isolation or detection of the complex formed between EDD and another protein. Binding of the antibody or ligand to a region of the first or second polypeptide that is not involved in complex formation is preferred for this purpose, the only requirement being that, in use, the ligand does not disrupt the complex formed.

Preferably, the ligand is a small molecule or alternatively, a binding partner for one of the protein complexes contemplated herein. Particularly preferred ligands for use in accordance with this embodiment are selected from the group consisting of a heat shock protein that binds to progesterone receptor, BRCA1, TP53, and nucleic acid (RNA or DNA).

The antibody or ligand may be labelled using a suitable reporter molecule, such as, for example, a fluorophore, chromophore, or radioisotope. In the case of antibodies and small molecules, these may also be detected using antibodies in accordance with procedures known to those skilled in the art.

Optionally, the kit is packaged with instructions for use.

The kits of the invention are useful for producing and/or detecting the protein complexes of the invention in vitro or in vivo. For producing the protein complexes, one or more of the non-antibody/ligand components of the kits is added to a cellular source for a time and under conditions sufficient for complex formation to occur. The antibody components are particularly useful for isolating the complex(es) thus formed. Optionally any one of the kit components is labelled with a protein tag to facilitate subsequent isolation or purification of the protein complex.

In use, the polypeptide components are contacted for a time and under conditions sufficient for complex formation to occur. Additional proteins may be provided from cellular or non-cellular sources to produce protein complexes other than those specifically referred to herein. When provided, the antibody or ligand is used to detect or isolate the complex formed. The ligand should be selected such that it does not disrupt the protein complex formed.

In another embodiment of the invention there is provided an isolated antibody that binds to a protein complex comprising an EDD protein, preferably a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor, subject to the proviso that said antibody does not bind to any individual protein of said complex in the absence of another protein of said complex.

Preferably, the antibody recognizes a conformational epitope of the protein complex.

Another embodiment of the present invention provides an anti-idiotypic antibody that binds to an antibody or ligand that binds to a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor, subject to the proviso that said anti-idiotypic antibody does not bind to an antibody that binds to an individual protein of said complex in the absence of another protein of said complex

Another embodiment of the present invention provides methods for isolating a EDD binding protein or a complex comprising same from a suitable cellular source.

Preferably, the protein or complex is provided substantially free of conspecific proteins, meaning that it is at least about 1-5% pure as determined by an analysis of proteins by SDS/PAGE. More preferably, the protein is at least about 10%, even more preferably at least about 20% pure, even more preferably at least about 25% pure, even more preferably at least about 30% pure, and even more preferably at least about 50% pure, and still more preferably substantially pure.

To isolate the protein complex of the invention, one or both binding partners are separately isolated, from the same or a different cellular source that ectopically expresses or endogenously expresses at least one of the said binding partners. The isolated binding partners are then combined in an amount and under conditions sufficient to facilitate their physical relation. Such conditions can be readily determined by those skilled in protein chemistry. Selection of buffer pH, ionic strength, and temperature, sufficient to maintain the binding partners in solution are generally preferred. One or more protease inhibitors can also be included to prevent proteolytic digestion or degradation of the isolated polypeptides.

Alternatively, the protein complex per se may be isolated from a cellular source or sub-cellular source (e.g. nuclei) that contains both binding partners endogenously or ectopically. It is within the scope of this embodiment that the binding partners are expressed as a fusion protein or as distinct polypeptides.

Preferred cellular sources of the isolated polypeptide binding partners, or the protein complex, include any mammalian cell, and preferably, a mammalian cell that is known to express EDD, and a protein selected from the group consisting of CHK2, BRCA2, CIB, importin alpha-1, importin alpha-3, importin alpha-5 and a progesterone receptor protein, or alternatively, a cell that can be engineered to express said protein(s). Exemplary cells for such a purpose include cancer cells (e.g. carcinoma cells, breast cancer cells such as ER-negative breast cancer cells, or squamous epithelial carcinoma cells or ovarian cancer cells, or hepatocellular carcinoma cells), epithelial cells, cells of the central nervous system, kidney cells, T cells, NIH3T3 cells, murine 10T fibroblasts, MDA-MB-231 cells, MDCK cells, COS cells, CHO cells, HeLa cells, or T-47D cells, HeLa cells, MCF-7 cells, or HEK 293 cells. The use of other cells (e.g. insect sf9 or sf21 cells, chick embryo cells and the like) is not excluded, particularly for isolation of a non-naturally occurring peptide, polypeptide or complex expressed by recombinant means.

Preferably, the protein complex or a binding partner thereof is isolated from cell line that endogenously expresses one or both binding partners, such as, for example, a cancer cell selected from the group consisting of head and neck cancer, melanoma, metastatic melanoma, a squamous cell carcinoma of the tongue, breast cancer, adenocarcinoma, squamous lung cancer, gastrointestinal cancer (eg. gastric, colon, or pancreatic cancer), ovarian cancer, hepatocellular carcinoma, renal cell cancer, bladder cancer, a gynecological carcinoma (eg. ovarian cancer), prostate cancer, squamous cell carcinoma, non-squamous carcinoma, glioblastoma and medulloblastoma. More preferably, the cell will be a metastatic melanoma cell, squamous cell carcinoma cell, a breast cancer cell, a hepatocellular carcinoma cell, or an ovarian cancer cell or a cell line derived from such cancers. In a particularly preferred embodiment, the protein complex or a binding partner thereof is isolated from a carcinoma cell or carcinoma cell line, HeLa cell, MCF-7 cell, T47D cell, or a HEK293 cell or COS cell that ectopically expresses one or more binding partners.

Means for isolating the peptide, polypeptide, or protein binding partners, or the protein complex, include any means of protein isolation known to the skilled protein chemist, such as, for example, size exclusion chromatography, ion-exchange (anion or cation exchange) chromatography, reverse phase chromatography, or affinity chromatography. Both high pressure (e.g. HPLC, FLPC, MALDI) and low pressure systems can be used.

Affinity methods using ligands or antibodies that bind to one or both of the binding partners to the protein-protein interaction are particularly preferred. Antibodies against a protein domain of an EDD protein or one of its binding partners are particularly useful for isolating EDD or a complex comprising same. In one embodiment, naturally-occurring or recombinant protein is purified free of conspecific proteins by providing a matrix comprising antibody coupled to activated chromatographic resin (eg. CNBr-activated Sepharose, Pharmacia), blocking the resin and washing to remove unbound antibody and blocking agent, contacting the resin with a protein extract comprising a peptide or polypeptide to which the antibody binds under conditions sufficient to allow binding of said peptide or polypeptide (e.g., high ionic strength buffers in the presence of detergent), and eluting said peptide or polypeptide under conditions that disrupt the antibody antigen binding (eg, a buffer of pH 2-3 or a high concentration of a chaotrope, such as urea or thiocyanate ion).

It will be apparent from the preceding description that small molecules, or proteins capable of binding to one of the binding partners, can also be used to isolate one or both binding partners, or the protein complex per se, by affinity means. Conditions to permit such isolation can be readily determined by those skilled in protein chemistry. Selection of buffer pH, ionic strength, and temperature, sufficient to maintain the binding partners in solution are generally preferred. Preferably, one or more protease inhibitors (e.g. papain, PMSF, leupeptin) are included to prevent proteolytic digestion or degradation of the isolated polypeptides. For example, naturally-occurring or recombinant protein is purified free of conspecific proteins by providing a matrix comprising a small molecule or protein binding partner coupled to activated chromatographic resin (eg. CNBr-activated Sepharose, Pharmacia), blocking the resin and washing to remove unbound material and blocking agent, contacting the resin with a protein extract comprising a peptide or polypeptide to which the antibody binds under conditions sufficient to allow binding of said peptide or polypeptide, and eluting said peptide or polypeptide under conditions that disrupt the binding.

In another embodiment, the invention provides methods for producing a protein complex described herein by recombinant means. For expressing peptides or polypeptides by recombinant means, a protein-encoding nucleotide sequence is placed in operable connection with a promoter or other regulatory sequence capable of regulating expression in a cell-free system or cellular system.

In one embodiment of the invention, nucleic acid comprising a sequence that encodes an EDD protein or a portion of an EDD protein and a protein selected from the group consisting of a protein having tumor suppressor activity, a protein having cell cycle modulatory activity, a protein associated with DNA repair or damage, a nuclear targeting protein, and a progesterone receptor protein or a portion of said polypeptide sufficient to bind to said EDD protein or said portion of an EDD protein, in operable connection with a suitable promoter sequence, is expressed in a suitable cell for a time and under conditions sufficient for expression to occur.

Nucleic acid encoding the binding partners is readily derived from the nucleotide and amino acid sequences set forth herein, which except for SEQ ID Nos: 3-7, are publicly available. To produce a fusion polypeptide, the open reading frames are covalently linked in the same reading frame, such as, for example, using standard cloning procedures as described by Ausubel et al. (Current Protocols in Molecular Biology, Wiley lnterscience, ISBN 047150338, 1992), which is herein incorporated by reference.

Optionally, a spacer is placed between the open reading frames of the binding partners to facilitate their physical relation. Preferred spacers comprise protein-encoding nucleotide sequences of at least about 15-30 nucleotides in length, preferably sequences encoding amino acids rich in proline. The spacer is designed such that it does not interrupt the open reading frames of the partners.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the Iacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley lnterscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6× His and MYC tag; and the retrovirus vector pSRatkneo (Muller et al., Mol. Cell. Biol., 11, 1785, 1991). The vector pcDNA 3.1 myc-His (Invitrogen) is particularly preferred for expressing a secreted form of a protein in 293T cells, wherein the expressed peptide or protein can be purified free of conspecific proteins, using standard affinity techniques that employ a Nickel column to bind the protein via the His tag.

A wide range of additional hosttvector systems suitable for expressing polypeptide binding partners or immunological derivatives thereof are available publicly, and described, for example, in Sambrook et al (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

In another embodiment, nucleic acid comprising a sequence encoding each binding partner is placed in operable connection with a promoter sequence and expressed in a suitable cell. If the protein partners are expressed in the same cell, they may freely associate in said cell to form the protein complex of the invention. If the protein partners are produced in different cells, the cells are lysed and the cellular lysates mixed under conditions sufficient to permit the association of the binding partners.

In accordance with this embodiment, the nucleotide sequences encoding the binding partners may be contained in the same or different nucleic acid molecules, and as a consequence, the use of single or multiple gene constructs to express the binding partners is clearly encompassed by the invention. The requirements for expressing fusion polypeptides as described herein above are also relevant in this context, except that there is no need for a spacer. Generally, different promoters will be used to express each binding partner, such as, for example, to prevent squelching or competition between promoters or regulatory sequences for cellular transcription factors.

Another embodiment of the present invention provides prognostic and diagnostic methods for determining a predisposition for disease, or a disease state, said methods comprising detecting a protein complex comprising:
(i) an EDD protein; and
(ii) a nuclear protein selected from the group consisting of a protein having tumor suppressor activity, a protein having cell cycle modulatory activity, a protein associated with DNA repair or damage, a nuclear targeting protein, a progesterone receptor protein and a protein associated with vascularization.

In one embodiment, the diagnostic/prognostic methods described herein detect a protein complex selected from the group consisting of (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor.

In one embodiment, the invention relates to reagents and methods for detecting specific interaction between an EDD protein and a nuclear protein having tumor suppressor activity wherein the protein-protein interaction is associated with unregulated cell division or hyperproliferation of a cell or the appearance or occurrence of tumors associated with cancer, such as, for example, ovarian cancer or hepatocellular carcinoma or squamous cell carcinoma or breast cancer or metastatic melanoma.

In another embodiment, the invention relates to reagents and methods for detecting specific interaction between an EDD protein and a nuclear protein having cell cycle modulatory activity wherein the protein-protein interaction is associated with unregulated cell division or hyperproliferation of a cell or the appearance or occurrence of tumors associated with cancer, such as, for example, ovarian cancer or hepatocellular carcinoma or squamous cell carcinoma or breast cancer or metastatic melanoma.

In another embodiment, the invention relates to reagents and methods for detecting specific interaction between an EDD protein and a protein associated with DNA damage or DNA repair wherein the protein-protein interaction is associated with unregulated cell division or hyperproliferation of a cell or the appearance or occurrence of tumors associated with cancer, such as, for example, ovarian cancer or hepatocellular carcinoma or squamous cell carcinoma or breast cancer or metastatic melanoma.

In another embodiment, the invention relates to reagents and methods for detecting specific interaction between an EDD protein and a progesterone receptor protein, wherein the protein-protein interaction activates the receptor in a ligand-dependent manner such as, for example, to enhance progesterone-mediated tumorigenesis or tumor growth or unregulated cell division or hyperproliferation of a cell.

In another embodiment, the invention relates to reagents and methods for detecting specific interaction between an EDD protein and a protein associated with vascularization, wherein the protein-protein interaction is associated with formation of new blood vessels in a subject. Preferably, the protein-protein interaction is associated with vasculogenesis or angiogenesis in a subject suffering from cancer, psoriasis, diabetic blindness, age related macular degeneration or rheumatoid arthritis.

In another embodiment, the invention relates to reagents and methods for detecting specific interaction between an EDD protein and a calcium/integrin binding protein (CIB) or DNA-dependent protein kinase interacting protein (KIP), wherein the protein-protein interaction is reduced in cells suffering DNA damage.

Preferred detection systems contemplated herein include any known assay for detecting a protein-protein interaction in a biological sample isolated from a human or mammalian subject, such as, for example, using one or more antibodies against the complex or each binding partner, or an epitope thereof. Alternatively, a non-antibody ligand of the protein complex may be used, such as, for example, a small molecule (e.g. a chemical compound, agonist, antagonist, allosteric modulator, competitive inhibitor, or non-competitive inhibitor, of the complex that may or may not modulate complex formation or dissociation).

The use of antibody-based assay systems is particularly preferred. In accordance with these embodiments, the antibody or small molecule may be used in any standard solid phase or solution phase assay format amenable to the detection of protein complexes or protein-protein interactions.

Antibodies that specifically bind to the protein complex are used for the diagnosis of conditions or diseases characterized by the presence of said protein complex, or in prognostic assays to monitor disease progression in the presence of absence of treatment. Diagnostic assays for include methods which utilize the antibody and a label to detect the protein complex in human body fluids or extracts of cells or tissues. The antibodies are used with or without modification, and may be labeled, either covalently or non-covalently, with a reporter molecule. A wide variety of reporter molecules which are known in the art may be used, several of which are described above.

A variety of protocols, including ELISA, RIA, and FACS, for measuring the protein complex are known in the art or described herein. Such methods provide a basis for diagnosing levels of the protein complex associated with disease. For example, a protein complex of the present invention that is associated with cancer induced by over expression of EDD and/or one of its binding partners, (e.g. EDD/CHK2 or EDD/BRCA2) can provide a poor prognosis of survival from the disease. Normal or standard values of the complex for a healthy individual are established by combining body fluids or cell extracts taken from normal or healthy subjects, preferably human subjects.

The amount of standard complex formation may be quantified by various methods, preferably by photometric means, or using antibodies in a quantitative immunoassay (e.g. ELISA), wherein the amount of protein complex is determined by comparison against known amounts of a standard peptide, such as, for example, a peptide comprising an EDD protein domain.

Quantities of the protein complex expressed in subject samples from biopsied tissues are compared with the standard values. Deviation between standard and subject values establishes the parameters for diagnosing disease or establishing a prognosis. In the case of cancerous tissues, a level of a protein complex in excess of the standard level of that protein complex detected in a healthy subject, is diagnostic of disease, and indicates a poor prognosis for survival.

Optical or fluorescent detection, such as, for example, using mass spectrometry, MALDI-TOF, biosensor technology, evanescent fiber optics, or fluorescence resonance energy transfer, is clearly encompassed by the present invention.

In biosensor diagnostic devices, the assay substrate and detector surface are integrated into a single device. One general type of biosensor employs an electrode surface in combination with current or impedance measuring elements for detecting a change in current or impedance in response to the presence of a protein-protein binding event (e.g. U.S. Pat. No. 5,567,301). Gravimetric biosensors employ a piezoelectric crystal to generate a surface acoustic wave whose frequency, wavelength and/or resonance state are sensitive to surface mass on the crystal surface. The shift in acoustic wave properties is therefore indicative of a change in surface mass, such as, for example, as a consequence of protein-protein binding (e.g. U.S. Pat. Nos. 5,478,756 and 4,789,804. Biosensors based on surface plasmon resonance (SPR) effects have also been proposed, for example, in U.S. Pat. Nos. 5,485,277 and 5,492,840, which exploit the shift in SPR surface reflection angle that occurs when protein binds to the SPR interface. Finally, a variety of biosensors that utilize changes in optical properties at a biosensor surface are known, (e.g., U.S. Pat. No. 5,268,305).

Biosensors have a number of potential advantages over binding assay systems having separate reaction substrates and reader devices. One important advantage is the ability to manufacture small-scale, but highly reproducible, biosensor units using microchip manufacturing methods, such as, for example, described in U.S. Pat. Nos. 5,200,051 and 5,212,050. Another advantage is the potentially large number of different analyte detection regions that can be integrated into a single biosensor unit, allowing sensitive detection of several analytes with a very small amount of body-fluid sample. Accordingly, the simultaneous detection of the individual binding partners that form the protein complex, or the simultaneous detection of one or more protein complexes of the present invention, is possible using a biosensor.

Evanescent biosensors are particularly preferred because they do not require separation of the protein complex from unbound material, and their use can be coupled to standard immunoassay formats, as originally described by Hirshfield in U.S. Pat. No. 4,447,546. In general, evanescent biosensors rely upon light of a particular wavelength interacting with a fluorescent molecule, such as, for example, a fluorescent antibody or small molecule attached near the probe's surface, to emit fluorescence at another wavelength, on binding of the protein complex of the invention to the antibody or small molecule. The biosensor is protected from sensitivity degradation caused by non-specific binding of proteins to the sensor surface, by exposing the sensor surface to a solution of non-interfering proteins, so that the non-interfering proteins bind to said sensor surface to prevent the subsequent binding of the interfering proteins. Enhanced protection of surfaces from biological proteins is also possible by completely covering surfaces with protective coatings, such as, for example, amorphous copolymers of tetrafluoroethylene and bis-2,2-trifluoromethyl-4.5-difluoro-1,2-dioxole, dissolved in a solvent containing fluorinated alkanes, and applied by deposition as a thin protective coating (U.S. Pat. No. 5,356,668 by Paton et al.).

Assay systems suitable for use in high throughput screening of mass samples, particularly a high throughput spectroscopy resonance method (e.g. MALDI-TOF, electrospray MS or nano-electrospray MS) or a detection system facilitating determination of real time association/dissociation constants, are particularly contemplated.

In an alternative embodiment, a diagnosis or prognosis is made by separately determining the level(s) of expression of the binding partners. In this case, the level of expression of the binding partners is determined by standard protein-based detection systems, antibody-based methods, or nucleic acid-based methods. For example, a high level of expression of certain binding partners such as EDD and a progesterone receptor can be indicative of disease, or, in the case of cancerous tissues, provide a poor prognosis for survival. Without being bound by any theory or mode of action, this may be a consequence of EDD trans-activating the progesterone receptor, thereby enhancing progestin-sensitive or progesterone-receptor mediated cell proliferation.

On the other hand, whilst low levels of CHK2 or BRCA2 coupled with high levels of EDD can be diagnostic of disease or provide a poor prognosis, low levels of EDD coupled with relatively high levels of CHK2 and/or BRCA2 are generally indicative of a good prognosis. Without being bound by any theory or mode of action, this may be a consequence of EDD preventing normal BRCA2/CHK2 function in cells.

In one embodiment, nucleic acid encoding EDD or a binding partner thereof, such as, for example, a synthetic oligonucleotide, complementary RNA, DNA, or protein-nucleic acid (PNA), is used to detect and quantitate gene expression in biopsied tissues in which expression of the polypeptide is correlated with disease. The diagnostic assay may be used to distinguish between absence, presence, and over expression of the binding partner, or to monitor expression following an initial diagnosis or during therapeutic intervention. As with protein detection systems, the detection of over expression of EDD and/or progesterone receptor is preferred.

Co-localization of expression of several binding partners in a particular cell, tissue or organ, such as, for example, using FISH or other expression detection system, is also indicative of disease.

In one embodiment, hybridization with PCR probes capable of detecting the nucleic acid (RNA or genomic DNA) encoding a binding partner is used. The specificity of the probe, is determined by Rs nucleotide sequence and the stringency of the hybridization or amplification (maximal, high, intermediate, or low). Generally, highly specific probes are preferred for use under more stringent conditions.

To provide a basis for the diagnosis of disease associated with expression of the binding partners, a normal or standard profile for expression of the partners is established, such as, for example, by combining body fluids or cell extracts taken from normal subjects with nucleic acid encoding the binding partners or a portion thereof, under conditions suitable for hybridization or amplification. Standard hybridization is then quantified by comparing the values obtained from normal subjects with the signal obtained using a known amount of a substantially purified nucleic acid. Standard values from normal samples are then compared with values from patient samples. Deviation between standard and subject values is diagnostic of the disease. Once a diagnosis is made by this or another method, hybridization assays are carried out to evaluate expression of the binding partners over time, or during a course of treatment.

With respect to cancer, the presence of relatively high amounts of RNA encoding EDD and/or its cognate binding partners, particularly the progesterone receptor, in biopsied tissue from an individual indicates a predisposition for the development of the disease, or is otherwise diagnostic of the disease, preferably prior to the appearance of actual clinical symptoms. Alternatively, or in addition, high levels of these transcripts can be indicative of a poor prognosis for survival.

Methods that are used to quantitate the expression include radiolabeling or biotinylating nucleotides, coamplification of a control nucleic acid, and the use of standard curves onto which the experimental results are interpolated (Melby et al., J. Immunol. Methods, 159, 235-244, 1993; Duplaa et al., Anal. Biochem. 212, 229-236, 1993).

Screening Assays for Identifying Modulators of Protein Complex Formation

A further embodiment of the present invention provides methods for determining a modulator of the activity, formation or stability of an isolated or recombinant protein complex comprising:
(i) an EDD protein or a portion of an EDD protein sufficient to bind to a protein selected from the group consisting of a protein having tumor suppressor activity, a protein having cell cycle modulatory activity, a protein associated with DNA repair or damage, a nuclear targeting protein, a progesterone receptor protein and a protein associated with vascularization; and
(ii) a nuclear protein selected from the group consisting of a protein having tumor suppressor activity, a protein having cell cycle modulatory activity, a protein associated with DNA repair or damage, a nuclear targeting protein, a progesterone receptor protein and a protein associated with vascularization or a portion of said protein sufficient to bind to said EDD protein or said portion of an EDD protein.

In one embodiment, the protein complex is selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor.

The modulator can enhance complex formation or stability or alternatively, partially or completely inhibits formation of the protein complex, prevent complex formation or enhance complex turnover in cells. In one embodiment, the modulator facilitates or enhances EDD and/or progesterone receptor turnover, particularly in cancer cells.

In their general form, the methods of the present invention comprise determining the association or dissociation of the protein complex, or the structure of the complex, in the presence and absence of a candidate compound or a candidate antibody. In accordance with the embodiment described herein, a modified association, dissociation, or structure, of the protein complex in the presence of a candidate compound or a candidate antibody indicates that the candidate is a modulator of the protein complex.

The association, dissociation, or structure of the complex may be determined by direct means, such as, for example, by determining real time association or dissociation constants in the presence and absence of the candidate, or modified binding of an antibody that recognizes a conformational epitope of the complex. Biosensors used essentially as described herein above, in the presence or absence of the candidate compound or antibody, are particularly suited to such applications.

Alternatively, the association, dissociation, or structure of the complex may be determined by indirect means, such as, for example, using a protein recruitment system, n-hybrid screen, reverse n-hybrid screen, plate agar diffusion assay, ELISA, or other well known assay format for detecting protein-protein interactions. Such indirect means generally use a reporter system to detect formation or dissociation of the protein complex.

Standard solid-phase ELISA assay formats are particularly useful for identifying antagonists of the protein-protein interaction. In accordance with this embodiment, one of the binding partners (e.g. EDD or a portion thereof) is immobilized on a solid matrix, such as, for example an array of polymeric pins or a glass support. Conveniently, the immobilized binding partner is a fusion polypeptide comprising Glutathione-S-transferase (GST; e.g. an EDD-GST fusion), wherein the GST moiety facilitates immobilization of the protein to the solid phase support. The second binding partner (e.g. progesterone receptor, CHK2, CIB, or BRCA2) in solution is brought into physical relation with the immobilized protein to form a protein complex, which complex is detected using antibodies directed against the second binding partner. The antibodies are generally labelled with fluorescent molecules or conjugated to an enzyme (e.g. horseradish peroxidase), or alternatively, a second labelled antibody can be used that binds to the first antibody. Conveniently, the second binding partner is expressed as a fusion polypeptide with a FLAG or oligo-histidine peptide tag, or other suitable immunogenic peptide, wherein antibodies against the peptide tag are used to detect the binding partner. Alternatively, oligo-HIS tagged protein complexes can be detected by their binding to nickel-NTA resin (Qiagen), or FLAG-labeled protein complexes detected by their binding to FLAG M2 Affinity Gel (Kodak). It will be apparent to the skilled person that the assay format described herein is amenable to high throughput screening of samples, such as, for example, using a microarray of bound peptides or fusion proteins.

In a modification of the standard ELISA-type assay format, a binding partner is immobilized on a solid support, such as by chemical synthesis thereon, or biotin-labelled and used in the liquid phase.

A two-hybrid assay is described in U.S. Pat. No. 6,316,223 to Payan et al., incorporated herein by reference. The basic mechanism described by Payan et al. is similar to the yeast two hybrid system. In the two-hybrid system, the binding partners are expressed as two distinct fusion proteins in a mammalian host cell. In adapting the standard two-hybrid screen to the present purpose, a first fusion protein consists of a DNA binding domain which is fused to one of the binding partners, and a second fusion protein consists of a transcriptional activation domain fused to the other binding partner. The DNA binding domain binds to an operator sequence which controls expression of one or more reporter genes. The transcriptional activation domain is recruited to the promoter through the functional interaction between binding partners. Subsequently, the transcriptional activation domain interacts with the basal transcription machinery of the cell, thereby activating expression of the reporter gene(s), the expression of which can be determined. Candidate bioactive agents that modulate the protein-protein interaction between the binding partners are identified by their ability to modulate transcription of the reporter gene(s) when incubated with the host cell. Antagonists will prevent or reduce reporter gene expression, while agonists will enhance reporter gene expression. In the case of small molecule modulators, these are added directly to the cell medium and reporter gene expression determined. On the other hand, peptide modulators are expressible from nucleic acid that is transfected into the host cell and reporter gene expression determined. In fact, whole peptide libraries can be screened in transfected cells.

Alternatively, reverse two hybrid screens, such as, for example, described by Vidal et al., Proc. Natl Acad. Sci USA 93, 10315-10320, 1996, may be employed to identify antagonist molecules. Reverse hybrid screens differ from froward screens supra in so far as they employ a counter-selectable reporter gene, such as for example, CYH2 or LYS2, to select against the protein-protein interaction. Cell survival or growth is reduced or prevented in the presence of a non-toxic substrate of the counter-selectable reporter gene product, which is converted by said gene product to a toxic compound. Accordingly, cells in which the protein-protein interaction of the invention does not occur, such as in the presence of an antagonist of said interaction, survive in the presence of the substrate, because it will not be converted to the toxic product. For example, EDD can be expressed as a DNA binding domain fusion, such as with the DNA binding domain of GAL4, and the portion of a progesterone receptor or CHK2 or BRCA2 that binds EDD is expressed as an appropriate transcription activation domain fusion polypeptide (e.g. with the GAL4 transcription activation domain). The fusion polypeptides are expressed in yeast in operable connection with the URA3 counter-selectable reporter gene, wherein expression of URA3 requires a physical relation between the GAL4 DNA binding domain and transcriptional activation domain. This physical relation is achieved, for example, by placing reporter gene expression under the control of a promoter comprising nucleotide sequences to which GAL4 binds. Cells in which the reporter gene is expressed do not grow in the presence of uracil and 5-fluororotic acid (5-FOA), because the 5-FOA is converted to a toxic compound. Candidate peptide inhibitor(s) are expressed in libraries of such cells, wherein cells that grow in the presence of uracil and 5-FOA are retained for further analysis, such as, for example, analysis of the nucleic acid encoding the candidate peptide inhibitor(s). Small molecules that antagonize the interaction are determined by incubating the cells in the presence of the small molecules and selecting cells that grow or survive of cells in the presence of uracil and 5-FOA.

Alternatively, a protein recruitment system, such as that described in U.S. Pat. No. 5,776,689 to Karin et al., is used. In a standard protein recruitment system, a protein-protein interaction is detected in a cell by the recruitment of an effector protein, which is not a transcription factor, to a specific cell compartment. Upon translocation of the effector protein to the cell compartment, the effector protein activates a reporter molecule present in that compartment, wherein activation of the reporter molecule is detectable, for example, by cell viability, indicating the presence of a protein-protein interaction. More specifically, the components of a protein recruitment system include a first expressible nucleic acid encoding a first fusion protein comprising the effector protein and one of the binding partners (e.g. progesterone receptor or a portion thereof or CHK2 or a portion thereof or BRCA2 or a portion thereof), and a second expressible nucleic acid molecule encoding a second protein comprising EDD with the NLS intact. The reporter molecule in this context would comprise a molecule or cellular event that is regulated by the effector protein. A cell line or cell strain in which the activity of an endogenous effector protein is defective or absent (e.g. a yeast cell or other non-mammalian cell), is also required, so that, in the absence of the protein-protein interaction, the reporter molecule is not expressed. In use, a complex is formed between the binding partner moiety of the fusion polypeptide and EDD, thereby directing translocation of the complex to the nucleus mediated by an importin protein binding to the EDD NLS, wherein the effector protein then activates the reporter molecule. Such a protein recruitment system can be practiced in essentially any type of cell, including, for example, mammalian, avian, insect and bacterial cells, and using various effector protein/reporter molecule systems.

Alternatively, a yeast cell based assay can be performed in which the interaction between EDD and one or more of its binding partners results in the recruitment of a guanine nucleotide exchange factor (GEF) to the plasma membrane, wherein GEF activates a reporter molecule, such as Ras, thereby resulting in the survival of cells that otherwise would not survive under the particular cell culture conditions. Suitable cells for this purpose include, for example, Saccharomyces cerevisiae cdc25-2 cells, which grow at 36° C. only when a functional GEF is expressed therein (Petitjean et al., Genetics 124, 797-806, 1990) Translocation of the GEF to the plasma membrane is facilitated by a plasma membrane localization domain. Activation of Ras is detected, for example, by measuring cyclic AMP levels in the cells using commercially available assay kits and/or reagents. To detect modulators of the protein-protein interaction of the present invention, duplicate incubations are carried out in the presence and absence of a 15 test compound, or in the presence or absence of expression of a candidate modulatory peptide in the cell. Reduced survival or growth of cells in the presence of a candidate compound or candidate peptide indicates that the peptide or compound is an antagonist of the interaction between EDD and one or more of its binding partners.

A “reverse” protein recruitment system is also contemplated, wherein modified survival or modified growth of the cells is contingent on the disruption of the protein-protein interaction by the candidate compound or candidate peptide. For example, NIH 3T3 cells that constitutively express activated Ras in the presence of GEF can be used, wherein the absence of cell transformation is indicative of disruption of the protein complex by a candidate compound or peptide. In contrast, NIH 3T3 cells that constitutively express activated Ras in the presence of GEF have a transformed phenotype (Aronheim et al., Cell. 78, 949-961, 1994)

In yet another embodiment, small molecules are tested for their ability to dissociate the protein complex of the invention, by an adaptation of plate agar diffusion assay described by Vidal and Endoh, TIBS 17, 374-381, 1999, which is incorporated herein by reference.

In a further embodiment, a modulator is determined by a process comprising:
(i) determining the level of a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor in the absence of a candidate compound or candidate antibody; and
(ii) determining the level of said protein complex in the presence of a candidate compound or in the presence of said candidate antibody
   wherein a difference in the level of said protein complex at (i) and (ii) indicates that the candidate compound or candidate antibody is a modulator of said interaction.

This embodiment of the invention applies mutatis mutandis to the determination of protein complexes comprising a portion of any one or more of the protein binding partners.

It will be understood by those skilled in the art that any one or more of the assay methods for antagonists as described herein above can be adapted for this purpose. This is because the level of the protein complex in the presence or absence of a candidate compound or antibody is related to antibody binding in the case of ELISAs, or to cell survival or growth, in the case of hybrid screens or protein recruitment assays. ELISA-based assay formats are particularly suitable for this purpose, because they are readily quantifiable, by calibrating the detection system against known amounts of a protein standard to which the antibody binds. Such quantitation is well known to the skilled person.

The modulators identified using the methods described herein are useful for the therapeutic or prophylactic treatment of hyperproliferative disorders, or disorders associated with aberrant cell cycle regulation, aberrant DNA damage or repair, aberrant vascularization, progestin-sensitive disorders or progesterone receptor-mediated disorders such as, for example, aberrant cell division, tumorigenesis, tumor metastasis, or tumor cell invasion. The modulators are preferably useful for the treatment of one or more symptoms associated with a cancer selected from the group consisting of squamous cell carcinoma, hepatocellular carcinoma, ovarian cancer, breast cancer, melanoma, head and neck cancer, adenocarcinoma, squamous lung cancer, gastrointestinal cancer (eg. gastric, colon, or pancreatic cancer), renal cell cancer, bladder cancer, prostate cancer, non-squamous carcinoma, glioblastoma and medulloblastoma. Alternatively, or in addition, the modulators are preferably useful for the treatment of disorders or conditions associated with aberrant vascularisation, such as those disorders associated with excessive vascularisation, eg some forms of aggressive cancer, diabetic blindness, age-related macular degeneration, rheumatoid arthritis and psoriasis, and those disorders associated with insufficient vascularisation, eg coronary artery disease, stroke, and delayed wound healing. Additionally, it is preferable that the modulators are useful for inducing wound healing, stimulating organ regeneration, stimulating follicle development in the corpus luteum, stimulating placental growth during pregnancy and stimulating embryonic growth during pregnancy.

Therapeutic Applications

In another embodiment, the present invention provides a method for treating a condition associated with elevated expression of an EDD protein in a cell, said method comprising administering an amount of a compound effective to reduce EDD expression in a cell.

In one embodiment, the condition associated with EDD over expression is a cancer, particularly a cancer selected from the group consisting of squamous cell carcinoma, hepatocellular carcinoma, ovarian cancer, breast cancer, melanoma, head and neck cancer, adenocarcinoma, squamous lung cancer, gastrointestinal cancer (eg. gastric, colon, or pancreatic cancer), renal cell cancer, bladder cancer, prostate cancer, non-squamous carcinoma, glioblastoma and medulloblastoma. However, it is to be understood that the inventive method is suitable for preventing any cell advancing into mitosis if administered at an appropriate time and in a suitable amount.

In one embodiment, the compound administered comprises nucleic acid, Preferably, the nucleic acid is an antagonist of EDD expression, such as, for example, an antisense nucleic acid, peptide nucleic acid (PNA), ribozyme, or interfering RNA, which is complementary, in whole or in part, to a target molecule comprising a sense strand, and can hybridize with the target molecule, in particular, EDD-encoding RNA. When introduced into a cell using suitable methods, such a nucleic acid inhibits the expression of the EDD gene encoded by the sense strand. Antisense nucleic acid, ribozymes (eg. Cech et al., U.S. Pat. No. 4,987,071; Cech et al., U.S. Pat. No. 5,116,742; Bartel and Szostak, Science 261, 1411-1418, 1993), nucleic acid capable of forming a triple helix (eg. Helene, Anticancer Drug Res. 6, 569-584, 1991), PNAs (Hyrup et al., Bioorganic & Med. Chem. 4, 5-23, 1996; O'Keefe et al., Proc. Natl Acad. Sci. USA 93, 14670-14675, 1996), interfering RNAs (Elbashir et al., Nature 411, 494-498, 2001; Sharp, Genes Devel. 15, 485-490, 2001; Lipardi et al., Cell 107, 297-307, 2001; Nishikura, Cell 107, 415-418, 2001) or small interfering RNAs (siRNA) may be produced by standard techniques known to the skilled artisan, based upon the sequences disclosed herein.

Preferably, the antisense nucleic acid, ribozyme, PNA, interfering RNA or siRNA comprises a sequence that is complementary to at least about 15-20 contiguous nucleotides of a sequence having at least about 80% identity to SEQ ID NO: 1 or SEQ ID NO: 3 (ie. it is complementary to EDD-encoding mRNA) and can hybridize thereto. For example, such antagonistic nucleic acid can be complementary to a target nucleic acid having the sequence of SEQ ID NOs:1 or 3 or a portion thereof sufficient to allow hybridization. Longer molecules, comprising a sequence that is complementary to at least about 25, or 30, or 35, or 40, or 45, or 50 contiguous nucleotides of EDD-encoding mRNA are also encompassed by the present invention.

As exemplified herein, the use of interfering RNA, particularly siRNA is preferred for down-regulating EDD expression in a cell, thereby inhibiting cellular proliferation and causing cells to accumulate in the G2/M phase of the cell cycle, or causing altered cell-cell contacts, altered cell shape and disorganisation. Such interfering RNAs generally comprise an RNA molecule having a region of self-complementarity capable of forming a double stranded RNA.

In one embodiment, a construct comprising an antisense nucleic acid, ribozyme, PNA, interfering RNA or siRNA, can be introduced into a suitable cell to inhibit EDD expression and/or activity therein. In another embodiment, such a construct can be introduced into some or all of the cells of a mammal. The antisense nucleic acid, ribozyme, PNA, or interfering RNA, inhibits EDD expression and the subsequent formation of deleterious protein-protein complexes involving an EDD protein. Accordingly, a cancer, or a hyperproliferative process that is mediated by EDD in the cell containing the construct is inhibited.

The use of antibodies that can inhibit one or more functions characteristic of a EDD protein, such as a binding activity, a signalling activity, and/or stimulation of a cellular hyperproliferative response, is also encompassed by the present invention. In one embodiment, antibodies of the present invention can inhibit binding of a ligand (i.e., one or more ligands) to a mammalian EDD protein and/or can inhibit one or more functions mediated by a mammalian EDD protein in response to ligand binding. In a particularly preferred embodiment, the antibodies can inhibit (reduce or prevent) the interaction of EDD with a natural ligand such a the progesterone receptor.

One or more agents can be administered to the host by an appropriate route, either alone or in combination with another drug. An effective amount of a nucleic acid or antibody agent having antagonist or agonist activity is administered. An effective amount is an amount sufficient to achieve the desired therapeutic or prophylactic effect, under the conditions of administration, such as an amount sufficient for inhibition or promotion of EDD function.

For the treatment of cancers it is particularly preferred to target the expression of EDD in one or more specific cells or tissues, such as, for example, a cancer tissue or cell, thereby ensuring that the active compound is delivered to that cell/tissue and does not inhibit cell proliferation generally. Antibodies recognizing tumor-specific antigens have been used to deliver cytotoxic drugs to tumors. Antibodies recognizing tumor-specific antigens can be conjugated to the active compound, however in the case of solid tumors, such immunoconjugates may be less effective in penetrating tumor tissue.

Arap et al., Science, 279, 377-380, 1998 blocked tumor growth indirectly by inhibiting angiogenesis using a peptide that localized to endothelial cells associated with human breast carcinoma xenografts. By conjugating the cytotoxic drug doxorubicin to the peptide, a selective destruction of blood vessels associated with tumors was observed. This, in turn, resulted in the necrosis of the tumor and an increase in the survivability of the tumor-bearing mice.

Hong et al., (published US Patent Publicaton No. 20020102265; U.S. Ser. No. 09/899,376) describe the isolation of a peptide (HN-1) that is specifically internalized by human squamous carcinoma , and solid tumor tissue cells, such as breast cancer cells. Accordingly, an anticancer compounds that targets an Edd gene expression product, can be conjugated to HN-1 and administered specifically to tumor tissue such as, for example, a squamous cell carcinoma (e.g. of the oral cavity, pharynx, throat, paranasal sinus, nasal cavity, larynx, thyroid, parathyroid, salivary gland, skin of the face, skin of the neck or cervical lymph node) such as a squamous cell carcinoma of the tongue or head and neck, breast cancer, glioblastoma, or astrocytoma. Such conjugates can be delivered by intravenous administration, intratumoral administration, subcutaneous administration, intraperitoneal administration or topical administration. In an additional specific embodiment the conjugate is administered by local, regional or systemic administration.

Alternatively, nucleic acid encoding an inhibitor of an EDD expression product, such as, for example nucleic acid encoding siRNA capable of targeting Edd gene over expression or the formation of an EDD-containing protein complex, is introduced to a subject in need of treatment and expressed therein operably under the control of a suitable tumor-specific promoter sequence. For example, an infectious recombinant viral vector expressing the RNA can be targeted to tumor cells through cellular surface receptors by genetic or biochemical modification of the viral surface. Alternatively, cancer cells are targeted at the transcriptional level using lineage-specific promoters that restrict expression of the effector gene to a tumor cell and any related normal cell derived from the same developmental lineage. Examples of tumor types that have been targeted in this manner include tumors of the colon, lung; breast, hepatocellular carcinoma and melanoma. Tumor-specific promoters/enhancers have also been used in a therapeutic approach called “virus-directed enzyme/prodrug therapy” (VDEPT), wherein tumor-killing efficacy can be enhanced with reduced side effects on normal cells (the so-called “bystander effect”). For example, the alpha-fetoprotein (AFP) promoter/enhancer cassette has been utilized to control E1 expression from an Adenoviral vector, to induce a virus-mediated oncolytic effect on hepatocellular carcinoma. Thus, a tumor specific replication competent adenoviral (TSRCA) vector comprising the alpha-fetoprotein promoter to deliver a gene encoding siRNA is particularly preferred. Alternatively, a variation of this system, the “Complementary-Adenoviral Vector System” as described in US Patent Publication No. 20020142989 may be employed.

Alternatively, or in addition, the compound effective in reducing EDD expression can be administered in the form of a liposome such as a cationic liposome.

A variety of routes of administration are possible including, but not necessarily limited to oral, dietary, topical, parenteral (e.g., intravenous, intra-arterial, intramuscular, subcutaneous injection), and inhalation (e.g., intrabronchial, intranasal or oral inhalation, intranasal drops) routes of administration, depending on the agent and disease or condition to be treated.

Formulation of an agent to be administered will vary according to the route of administration selected (e.g., solution, emulsion, capsule). An appropriate composition comprising the agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The present invention clearly excludes any previously disclosed isolated protein complex consisting solely of EDD protein and TopBP1.

The present invention is further described by the following non-limiting Examples.

### EXAMPLE 1

### Abnormalities of the EDD Gene in Human Cancer

1.1 Materials and Methods

Tumors and DNA Extraction

Ovarian tumor tissue and matched blood or normal ovarian tissue were obtained from 98 patients and DNA extracted as described previously (Obata et al, Cancer Res. 58, 2095-2097, 1998). Metastatic melanoma tissue and matched blood or normal skin tissue were obtained from 20 patients and DNA isolated as reported previously (Indsto et al, Cancer Genet Cytogenet 100, 68-71, 1998). DNA was extracted from matched normal and hepatocellular carcinoma tissue samples microdissected from 19 primary liver tumors (Macdonald et al, Hepatology 28, 90-97, 1998). For AI analysis, paraffin-embedded breast cancers and normal blood were obtained from 24 patients. Tumor location was determined by haemotoxylin and eosin staining and cells were microdissected from 4-5 adjacent sections. DNA was extracted in lysis buffer (0.45% Tween 20, 5 mg/ml proteinase K, 0.25% BSA) at 55° C. for 8 hours, then boiled for 10 min. DNA was extracted from blood using a Puregene DNA isolation kit (Gentra Systems, Minneapolis, Minn.).

For RT-PCR breast cancer samples were collected at the time of surgery. Normal breast tissue (based on histological examination) was removed from unaffected regions of the breast at the same time as excision of the cancer.

Paraffin-embedded archival samples from 12 squamous cell carcinoma of the anterior tongue and matched lymph nodes were from a series described by Bova et al (Bova et al, Clin. Cancer Res. 5, 2810-2819, 1999). Areas of malignant squamous cells were identified by a pathologist from haemotoxylin and eosin stained slides and were microdissected from unstained adjacent 10 μm sections under a light microscope. Normal tissue was gained from uninvolved lymph nodes and/or microdissected from areas of normal cells surrounding the squamous cell carcinoma. Samples were digested in 250 μl of proteinase K (2 mg/ml) for 5 days at 37° C. with constant agitation. The digest was then extracted once with phenol, once with chloroform, and DNA precipitated in ethanol overnight and re-dissolved in 30 μl of TE buffer (“microdissected DNA”).

Isolation and Cloning of EDD-Linked Microsatellites CEDD and 586F18b.

A human P1 genomic library (Incyte Genomics, CA) was screened with two cDNA probes covering 4 kb of the EDD coding sequence (Callaghan et al, Oncogene 17, 3479-3491, 1998). DNA from positive clones was extracted, digested with Hinc II, separated by gel electrophoresis and re-screened by Southern blotting using an oligonucleotide probe (CA),₁₅. Positive genomic DNA fragments were cloned into pBluescript and sequenced which identified a novel dinucleotide repeat microsatellite CEDD (CA repeat near EDD). Unique primers were designed around the microsatellite to give a product of approximately 220 bp; CEDD forward 5′-TACCCTGCAGTAAATCTCACATGTACTCCC-3′ (SED ID NO: 5), CEDD reverse 5′-AGMTCGCTTGMCCTAGTAGGTGMGGTG-3′ (SED ID NO: 6). Subsequently, EDD genomic sequence became available (Genbank Accession: AC021004) and CEDD was located in an intron between bases 2616 and 2617 of the EDD coding sequence. The minimum size of the EDD gene is 100 kb, consisting of at least 46 exons. Within the same genomic clone another EDD-specific dinucleotide repeat microsatellite was identified and named 586F18b. Unique primers were designed around this microsatellite to give a product of approximately 200 bp; forward 5′-GCTAGGGMCCAAACTGCCAG-3′(SEQ ID NO: 24), reverse 5′-TGCAAAATMCMTAGCTTTGCTTAG-3′ (SEQ ID NO: 25). 586F18b is located in an intron between bases 6631 and 6632 of the EDD coding sequence. Both microsatellites were between 50-55% heterozygous using human cell line DNA (data not shown).

Microsatellite Analysis

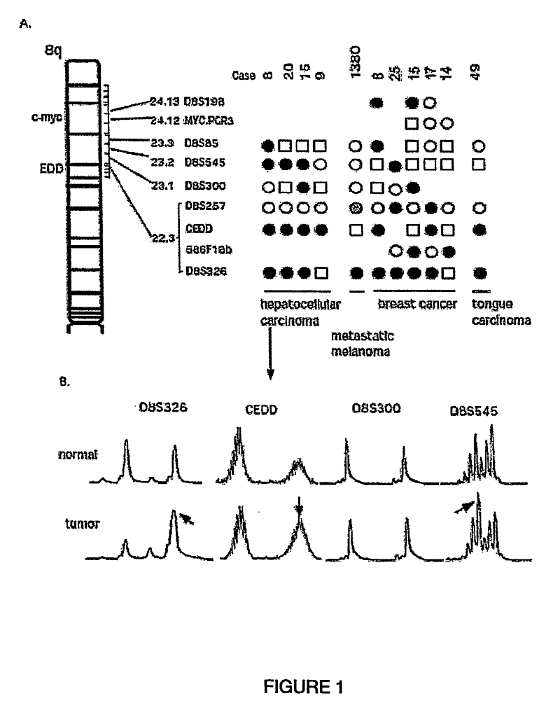

Microsatellite analysis was used to determine the frequency and distribution of AI on 8q22.3-24.13 using CEDD, 586F18b and seven other dinucleotide and tetranucleotide repeat polymorphic markers mapped in this region (Genome Database, www.gdb.org/) (FIG. 1). CEDD, D8S326, D8S257, D8S300, D8S545 and D8S85, were used for analysis of ovarian cancers, hepatocellular carcinoma, metastatic melanoma and squamous cell carcinoma of the tongue. The additional markers 586F18b, MYC-PCR.3 and D8S198 were used for analysis of breast cancers. Primer pairs were obtained from Research Genetics (Huntsville, Ala.) and Pacific Oligos (Lismore, Australia). Ovarian DNA was analyzed by radio-isotope based methods as previously described (Obata et al, Cancer Res. 58, 2095-2097, 1998). For DNA from other tissues, in each PCR reaction the forward primer was labeled with either 6-FAM or TET fluorescent label. Reactions were performed using 40-60 ng of DNA from hepatocellular carcinoma and metastatic melanoma, 1 μl of breast cancer extracted DNA or 2-3 μl microdissected DNA from squamous cell carcinoma of the tongue. The PCR reaction components were as follows: 10 mM Tris-HCl, pH 8.3; 50 mM KCl; 66 μM dNTP mix; 1.5 mM MgCl₂; 5 pmoles of both forward and reverse primer and 0.8 U of Amplitaq Gold DNA polymerase (Applied Biosystems, Sydney, Australia). PCR conditions for microsatellites D8S326, D8S257, D8S545, D8S85, D8S198 and MYC-PCR.3 were: 12 min hotstart 95° C.; 35 cycles (40 for microdissected DNA) of (1) 94° C. 15 s, (2) 60° C. 15 s (66° C. for CEDD, 52° C. for 586F18b) and (3) 72° C. 30 s; 5 min 72° C.; 4° C. hold. Amplification of the CEDD microsatellite from microdissected DNA required a 30 s annealing step, a 1 min extension step, and 35 cycles. Amplification of the microsatellite D8S300 was performed as follows: 12 min hotstart 95° C.; 35 cycles of (1) 94° C. 30 s, (2) 60° C. 30s and (3) 72° C. 60s; 5 min 72° C.; 4° C. hold. No PCR products could be obtained from DNA recovered from paraffin embedded tongue carcinoma with D8S300 primers, presumably because of DNA shearing.

Duplicate fluorescent products were separated on an ABI 377 DNA Sequencer (Applied Biosystems) and analyzed using Genescan and Genotyper software (Applied Biosystems). Ambiguous results were resolved on separate gels. AI in hepatocellular carcinoma and breast cancer was defined by a reduction in relative fluorescence of heterozygous allele peaks by at least 30% in the tumor DNA compared to the matched normal DNA. A reproducible difference of 20-30% in metastatic melanoma was considered significant due to the higher level of contaminating normal DNA present in these samples. A difference of 50% or more was considered to represent AI in squamous cell carcinoma DNA due to a lower level of normal cellular DNA contamination in these samples.

Allelic imbalance (AI) is indicative of either loss or amplification of an allele and (presumably) the surrounding chromosomal region. In practise, loss of heterozygosity (LOH) is not readily distinguishable from amplification by this method, particularly when there is a significant degree of cross-contamination of tumor with normal DNA. In the present study, AI was interpreted as amplification, given that chromosomal gains are much more commonly reported at 8 q than losses.

FISH

FISH analysis was carried out by Erica Woollaft at the Women's and Children's Hospital, Adelaide, Australia. Two P1 plasmids encoding approximately 100 kb of EDD genomic sequence used for fluorescence in situ hybridization (FISH) were nick-translated with biotin-14-dATP and hybridized at a final concentration of 20 ng/μl to metaphases from the breast cancer cell lines MDA-MB-436, SKBR-3, BT-20 and BT-483. The single copy FISH method was modified from that previously described (Callen et al, Ann. Genel. 33, 219-221, 1990) in that chromosomes were stained before analysis with both propidium iodide (as counterstain) and DAPI (for chromosome identification). Images of metaphase preparations were captured by a CCD camera using the ChromoScan image collection and enhancement system (Applied Imaging, Newcastle, UK).

Quantitative RT-PCR—Primary Breast Tissue.

RNA was extracted from samples using Trizol reagent (Life Technologies, Paisley Scotland, UK). Reverse transcription was performed, followed by quantitative PCR using a Taqman based methodology as previously described (Al-Taher et al, Yeast 17, 201-210, 2000). PCR primers for EDD were designed within 300 bases of the polyA addition site. The sequences of EDD specific primers were: forward EDD-407F GCTAGTCACCMCTTCTGGGTCTM (SEQ ID NO: 26), reverse EDD-49OR CAGCAAAAAGATAAATCACAGTGTAAATT (SEQ ID NO: 27), fluorescent probe EDD-433T FAM-CCCAGCCAAAGATGACAGCAGAACAAC-TAMRA (SEQ ID NO: 28). Samples were also analysed for expression of several control genes; CK18 (epithelial content), GAPDH (cellular metabolism), IF2B (general transcriptional activity) and MCM3 (proliferation rate). Results are expressed as copies of EDDI3e9 total cDNA per sample, corrected for IF2B.

Quantitative PCR and RT-PCR—Breast Cancer Cell Lines.

Cells harvested from duplicate 150 cm² flasks were pooled and RNA was extracted using the RNeasy RNA extraction kit (Qiagen, Melbourne, Australia). cDNA was made as previously described (see EDD mRNA sequence analysis) from the normal breast epithelial cell lines 184 and the breast cancer cell lines MDA-MB468, MDA-MB436, MDA-MB-231, MDA-MB453, MDA-MB-175, MDA-MB-361, MDA-MB-157, BT-20, T47D, BT-549, BT483, MCF-7, BT-474, SK-Br-3, ZR-75-1, Hs 578T, MDA-MB-330. All PCR reactions were run on a LightCycler using LightCycler software 3.5 (Roche Diagnostics, Sydney Australia). Primers for EDD forward, TTAGGCTTTTGGTAAATGGCTGCG (SEQ ID NO: 29), reverse, TGAGGGCATAGGCTGGMTCCTTC (SEQ ID NO: 30), carbonic anhydrase II, forward CCACCCCTCCTCTTCTGGMTG (SEQ ID NO: 31), reverse GCTTTGATTTGCCTGTTCTTCAGTG (SEQ ID NO: 32), p53 ribonucleotase reductase (p53R2), forward GTGACTTTGCTTGCCTGATGTTC (SEQ ID NO: 33), reverse TCTGTGGTTTCTGCCATMCTGC (SEQ ID NO: 34), GAPDH, forward GACATCMGAAGGTGGTGM (SEQ ID NO: 35), reverse TGTCATACCAGGAAATGAGC (SEQ ID NO: 36). All primer pairs spanned at least one intron and products were visualized on an agarose gel to confirm product size. Relative expression of all genes was corrected for cDNA concentration using GAPDH expression.

DNA was extracted from the above cell lines using a DNA extraction kit (Stratagene, Sydney, Austalia). PCR reactions to determine genomic copy number utilized the reverse primer from above (unless mentioned). Primers for EDD, forward CATTGCTGACCCTATCCCTGTGTTG (SEQ ID NO: 37), reverse TAGCCCGTGAAATCCTCCCATCTC (SEQ ID NO: 38), CA II forward ACCCGCCTCATGCCTCAGCCTTAC (SEQ ID NO: 39), p53R2 forward TGTCAGCCTTGAGTACCTCCAGGG (SEQ ID NO: 40), beclin forward TAGGTTTGGGGTGAGTGG (SEQ ID NO: 41), reverse AGTCTGTGGGCAGCMGG (SEQ ID NO: 42). All products were visualised on an agarose gel to confirm product size. Relative DNA copy number was corrected using known beclin gene copy number determined by FISH (Aita et al, Genomics 59, 59-65, 1999).

Gene Expression Profiling—Primary Ovarian Tissue

RNA was isolated from 66 primary ovarian cancers and borderline tumors in addition to 4 normal ovary samples using Trizol reagent (Life Technologies, Rockville, Md., USA) essentially according to manufacturer's instructions. RNA was then reverse transcribed using an oligo(dT) anchored oligonucleotide that additionally comprised a T7 promoter sequence. Isolated cDNA was then transcribed in vitro using the T7 MEGAscript kit (Ambion, Austin, Tex., USA) according to manufacturer's instructions. Transcription was performed with biotinylated nucleobdes (Bio-11-CTP and Bio-16-UTP) to facilitate detection of the transcribed nucleic acid.

Levels of gene expression in the cancer samples was then determined by analyzing the transcribed cDNA samples using customized Affymetrix GeneChip® microarrays that comprise 59,618 oligonucleotide probe sets. These probe sets facilitate analysis of 46,000 gene clusters, representing over 90% of the predicted expressed genome.

Data was normalized, and changes in gene expression detected using a ranked penalized t-statistic with p-values adjusted for multiple testing using the Holm procedure. Analysis was performed using the LIMMA package (available from Bioconductor, Biostatistics Unit of the Dana Farber Cancer Institute at the Harvard Medical School/Harvard School of Public Health).

Gene expression in 52 different tissues of the body was also determined using the previously described methods to facilitate the identification of changes in gene expression that are specific for ovarian cancer.

EDD mRNA Sequence Analysis.

Total RNA was extracted from the following human cell lines using the RNeasy Maxi Kit (Qiagen): breast cancer MDA-MB-468, MDA-MB436, MDA-MB-231, MDA-MB-453, MDA-MB-175, MDA-MB-361, MDA-MB-134, MDA-MB-157, BT-20, T-47D, BT-549, BT-483, MCF-7, BT474, SK-Br-3, ZR-75-1, Hs 578T; normal breast epithelium HBL-100 (SV-40 transformed), 184, HMEC 219-4; ovarian cancer OVCAR-3, OVCA-420, IGROV-1, SKOV3, A2780; prostate cancer PC-3, DU-145, LnCaP. cDNA was synthesized using the Expand Reverse Transcriptase System (Boehringer Mannheim, Sydney, Australia). 2 μg total RNA and 2 μl oligo dT (Boehringer Mannheim) was made up to 22 μl with water, heated at 65° C. for 10 min and cooled on ice followed by addition of 8 μl Expand Reverse Transcriptase buffer (5×; 250 mM Tns-HCl, 200 mM KCl, 25 mM MgCl₂, 2.5% Tween 20 (v/v), pH 8.3), 1 mM each dATP, dCTP, dGTP and dTTP, 10 mM DTT and 2 μl (100 U) Expand Reverse Transcriptase. The reverse transcriptase reaction was then performed at 42° C. for 45-60 min. Ten PCR products were designed to cover the entire 8.5 kb coding region of the EDD gene. PCR reactions included 3 μl of the reverse transcriptase reaction, 10 pmol of each primer, 1.75 units Expand High Fidelity DNA polymerase (Boehringer) and 1.5 mM MgCl₂. Amplification was carried out using the following protocol: 2 min denature at 94° C.; 10 cycles of 30 s denature, 30 s annealing and 60 s extension at 72° C.; 24 cycles of 30 s denature, 30 s annealing and 60 s extension with a 5 s increase per cycle; 5 min extension. Annealing temperatures depended on the primers used (primer sequences available on request). PCR products were purified using the QlAquick PCR purification system (Qiagen) and quantitated by visualization on a gel. Sequencing reactions were performed at the Australian Genome Research Facility (Brisbane, Australia) and sequence analysis and assembly were performed using Editview and Autoassembler software, respectively (Applied Biosystems).

SSCP

SSCP analysis was performed as previously described (Campbell et al, Hum. Mol. Genetics 3, 589-594, 1994) on 8 exons of EDD encoding regions of potential functional significance, i.e. the HECT domain, nuclear localization signal and zinc finger motif. Primers were designed within intronic sequence to generate PCR products between 200-300 bp).

EDD Immunohistochemistry

Immunohistochemistry (IHC) was performed on paraffin-embedded, formalin-fixed breast (9 normal breast and 46 breast cancers), general ovarian tissues (94 ovarian cancers) and 165 serous ovarian cancer tissues. Paraffin-embedded embryonic neural tissues from wild-type and EDD^{4 -/-}(knockout) mice were used as positive and negative controls, respectively. Paraffin-embedded cell pellets from the WT-30 cell line, which overexpresses EDD (Henderson et al., 2002) were used as an additional positive control.

Sections were dewaxed and rehydrated before unmasking in target retrieval solution (high pH: DAKO Corporation, Carpenteria, Calif.) in a waterbath, at 100° C. for 30 min.

Using a DAKO autostainer endogenous peroxidase activity was quenched in 3% hydrogen peroxide in methanol, endogenous avidin and biotin were blocked with an avidin biotin block (DAKO Corporation) and non-specific binding of secondary antibody was blocked by incubation with a serum free protein block (DAKO Corporation). Sections were incubated for 30 min with 1:150 anti-EDD (M19) antibody (Santa Cruz Biotechnology, Santa Cruz, Calif.) and, subsequently for 15 min with 1:200 biotinylated horse anti-goat (Vector Laboratories, Burlingame, Calif.). A streptavidin-biotin peroxidase detection system was used according to the manufacturer's instructions (Vectastain Elite kit; Vector Laboratories) with 3,3′-diaminobenzidine as substrate. Counterstaining was performed with Mayer's hematoxylin (DAKO Corporation).

The degree of staining was assessed by two separate observers and discrepancies were resolved by conferencing. EDD expression scores were determined by combining the percentage of cells expressing EDD (range of 1-4 for 1%-100% cells stained) and intensity of staining (range of 0-3). A combined score of 0 was called no expression, a score in the range of 1-5 was low expression and a score of 6 and 7 was called high expression.

The relative EDD expression and the proportion (percentage) of EDD positive cells in the serous ovarian cancer tissues were correlated with various clinicoathological parameters of ovarian disease (as shown in Table 2) in order to determine whether or not EDD expression is a prognostic marker of outcome of serous ovarian cancer.

1.2 Microsatellite Analysis

The frequency of AI surrounding the EDD locus (8q22.3) was investigated in malignant and pre-malignant ovarian tumors, breast cancers, hepatocellular carcinoma, metastatic melanoma and squamous cell carcinoma of the anterior tongue using nine microsatellites shown with their locations in FIG. 1. As CEDD and 586F18b are located within introns of the EDD gene it can be assumed that chromosomal loss or gain involving these alleles is equivalent to loss or gain of an EDD allele.

Within the complete cancer set (excluding non-malignant ovarian tumors) the frequency of AI in the narrow region of 8q under study was considerable: 60% (83/139 informative cases) had AI at one or more markers. The individual frequencies (cases with AI/informative cases) were: D8S326 47/103 (46%); CEDD 38/90, (42%); D8S257 29/95 (31%);D8S300 27169 (39%); D8S545 28/87 (32%); and D8S85 24/90 (27%) (Table 1). Notably, CEDD and the neighboring 8q22.3 marker D8S326 had the greatest frequency of AI. In addition, as can be seen from the following data, 30-40% of those cancers that have chromosomal aberrations at the CEDD microsatellite show no involvement of one or more microsatellites at the telomeric end of the region studied. This indicates that AI at the EDD locus often occurs independently of more extensive aberrations in these cancers (ie loss or gain of the whole chromosome arm or co-amplification with the MYC oncogene).

1.3 Ovarian cancer

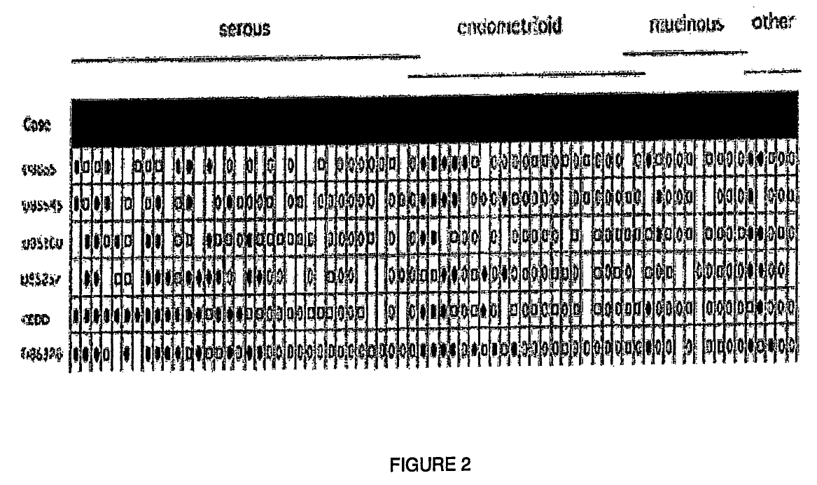

The ovarian tumor set was comprised of several cancer subtypes (predominantly mucinous, endometrioid and serous), borderline and non-malignant benign tumors. Within the cancer group 48% (34/71) displayed AI at one or more markers in the region (FIG. 2). In marked contrast, benign and borderline tumors exhibited only 1/23 and ⅕ cases of AI, respectively, involving several microsatellites but not CEDD or DS8257 (Table 1). Because of the low frequency of involvement of 8q22.3-8q23.3 in benign and borderline tumors they were omitted from the following analysis.

Several cancers had AI across all informative markers, consistent with large chromosomal aberrations eg cases 63, 114, 154. However, of interest are those cancers where AI is present at the EDD locus but not at the more telomeric markers (eg cases 14, 22, 32, 211, FIG. 2). This suggests that an important region of chromosomal aberration is located close to the EDD locus. Indeed a central finding of this study is the observation in serous cancers that CEDD was the microsatellite most commonly affected by AI ( 16/22, 73% of informative cases). This very high frequency of involvement differs significantly from that for the more telomeric markers D8S85 ( 6/18, 33%, p<0.01) or D8S845 ( 4/13, 31%, p<0.01). In fact, when serous cancers are compared to other ovarian cancers, CEDD is the only microsatellite for which AI differs significantly between the two groups (p=0.0018). However, no correlation was apparent between AI at any locus and cancer grade and stage either within the entire set of cancers or the serous subtype (data not shown).

Analysis of gene expression profiles of various ovarian cancers (both serous and epithelial) to identify those changes in gene expression that correlate with disease outcome following laparotomy (ie the time at which the gene expression profile is determined), demonstrates that changes in EDD gene expression in subjects is significantly associated with the survival of a subject suffering from ovarian cancer (ie p=0.00). Accordingly, EDD is clearly a prognostic marker of recovery from ovarian cancer following a laparotomy.

1.4 Hepatocellular Carcinoma, Squamous Cell Carcinoma of the Tongue and Metastatic Melanoma

In all three cancer types AI was common (Table 1) with 74%, 33% and 47% of cancers displaying AI of at least one microsatellite respectively.

In nineteen hepatocellular carcinomas four cancers had regions of imbalance that included CEDD but did not extend continuously telomeric to 8q22.3 (FIG. 1A). Similarly analysis of seventeen metastatic melanoma (Table 1) found three cancers where the region of AI did not involve markers telomeric to 8q22.3 (example shown in FIG. 1). Of the twelve squamous cell tongue carcinoma analyzed all had AI involving 8q22.3 (CEDD and/or D8S326) and in three of these cancers the region of imbalance did not extend to the most telomeric markers, D8S85 and D8S545 (example shown in FIG. 1). Thus in these three cancer types the chromosomal region at he EDD locus is commonly and often specifically aberrant.

1.5 Breast Cancer

For microsatellite analysis of breast cancer DNA, three additional microsatellites were introduced to provide more information about AI at the EDD locus (586F18b) and at the telomeric region of 8q around MYC (8q24.12) (D8S198 and MYC-PCR.3). As with the other cancer types studied, AI was common on 8q with 16/24 (67%) breast cancers displaying AI at one or more markers (Table 1). CEDD or 586F18b were involved in 6/16 (38%) of informative cases. Commonly, AI involved MYC-PCR.3 or D8S198, consistent with the frequent amplification of MYC in breast cancer, but in the majority of cancers AI was not continuous from 8q22.3 to 8q24 (examples shown in FIG. 1).

1.6 EDD mRNA Expression in Breast Cancers

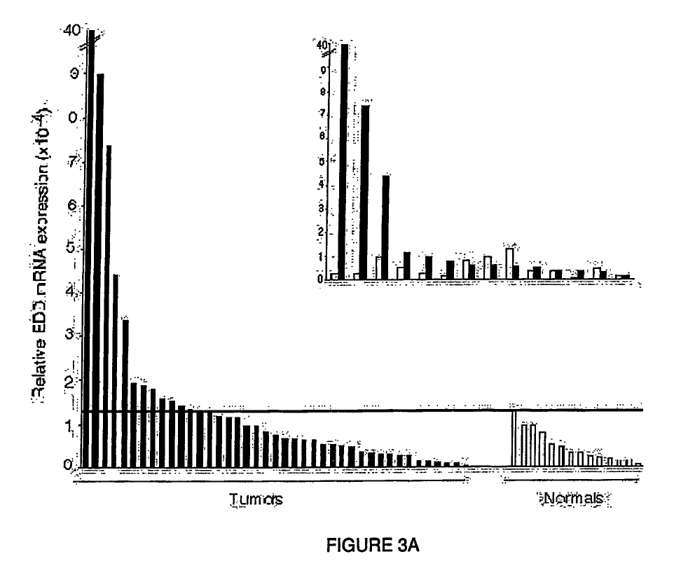

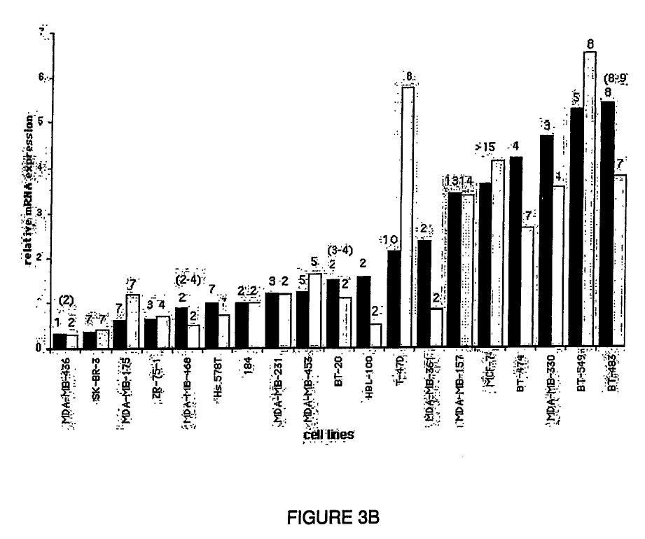

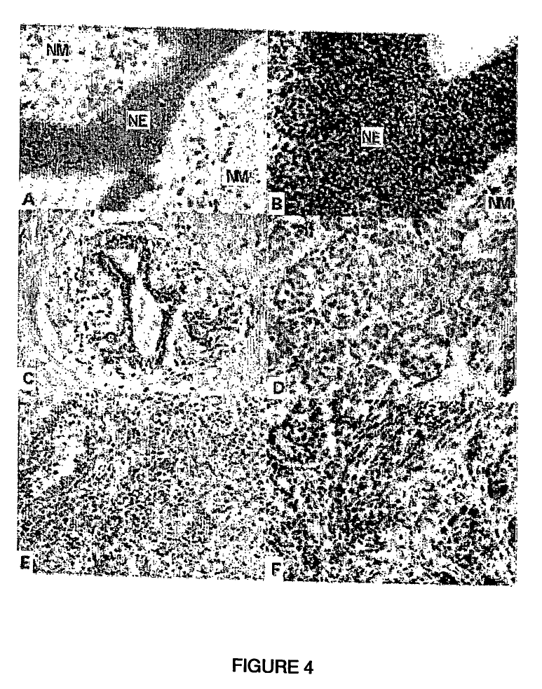

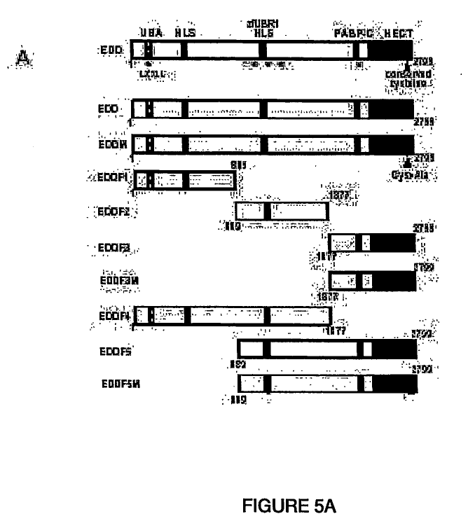

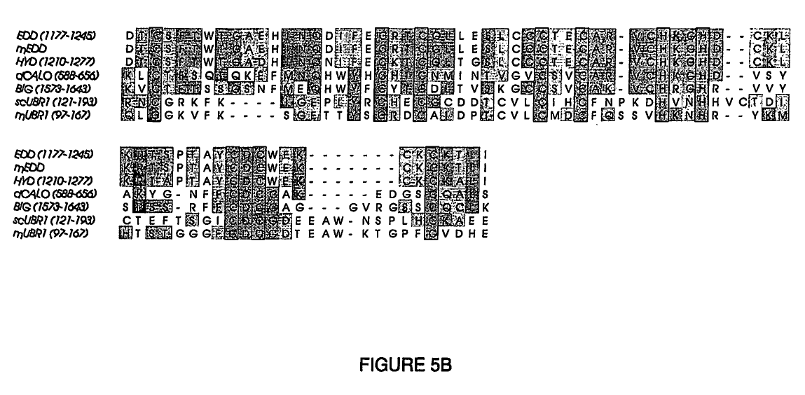

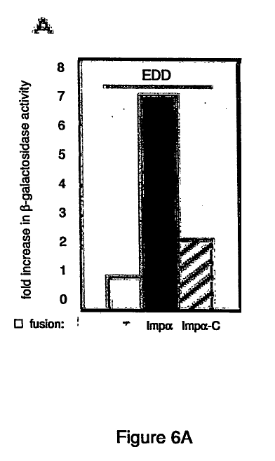

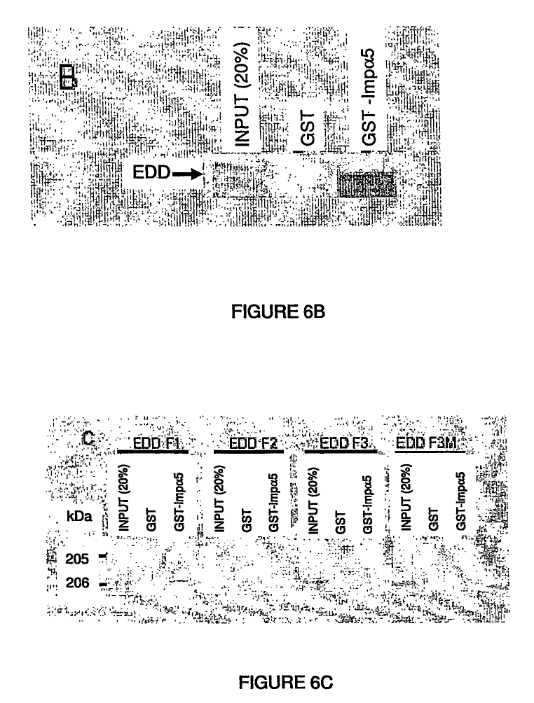

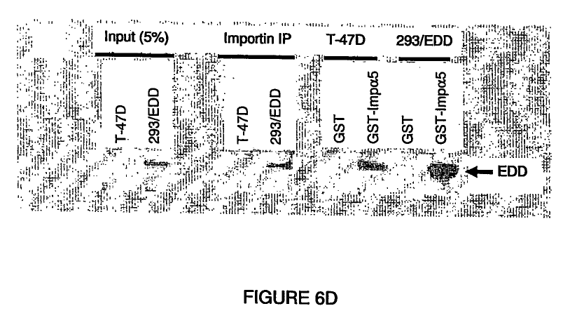

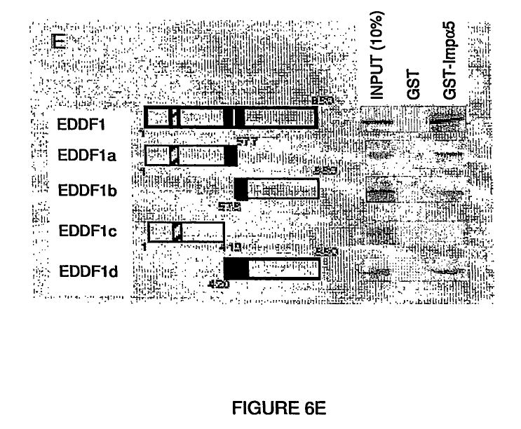

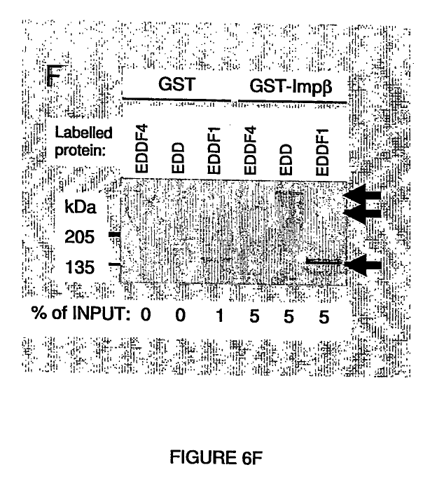

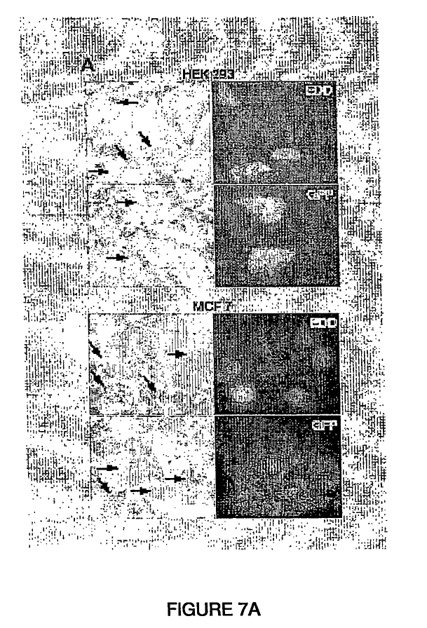

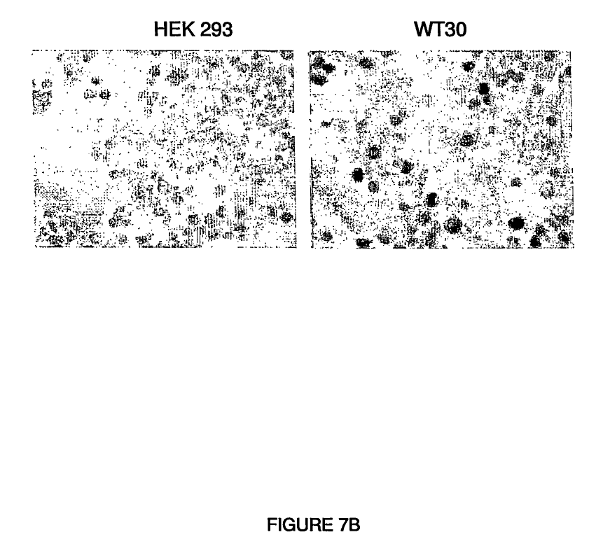

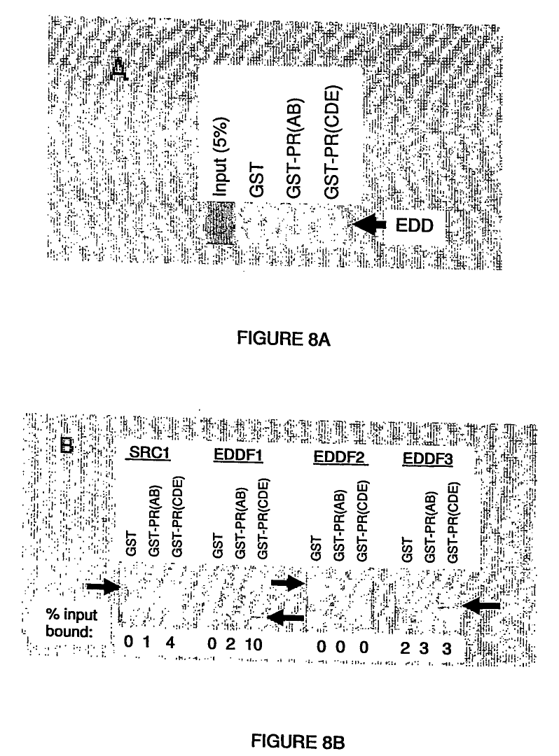

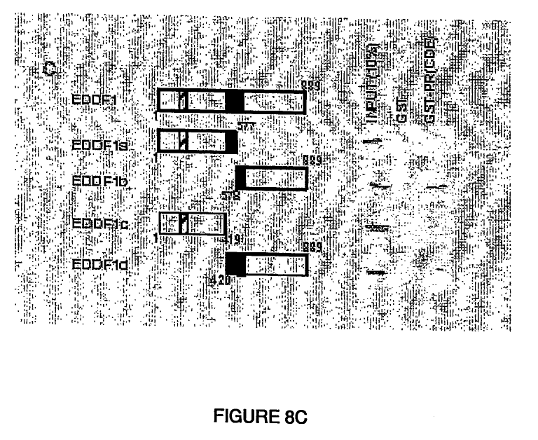

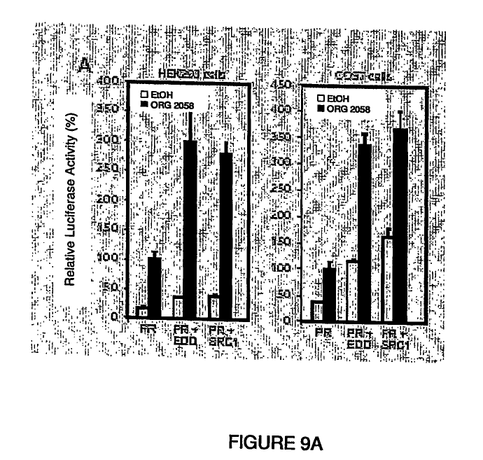

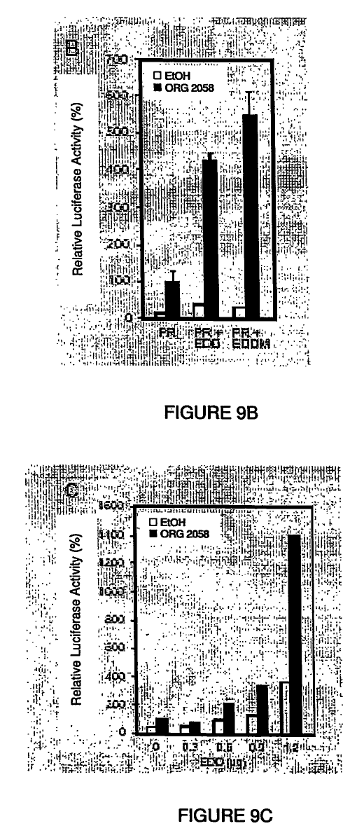

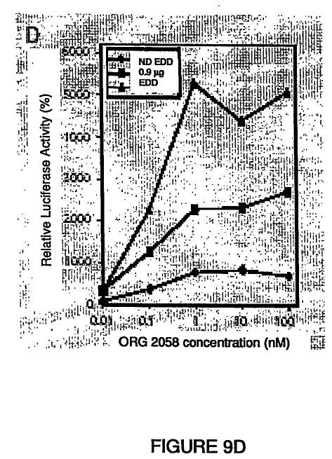

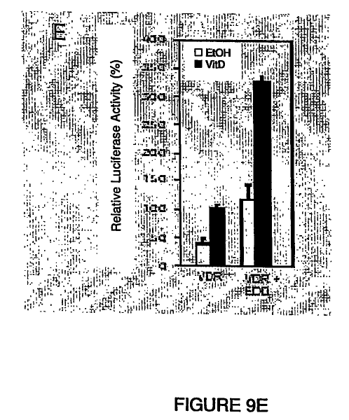

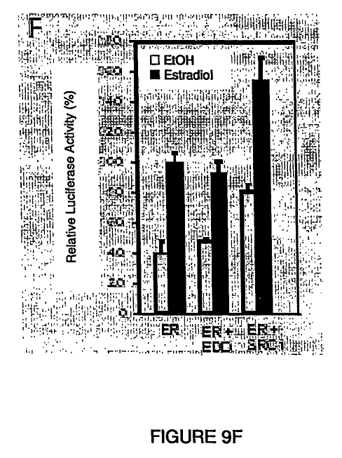

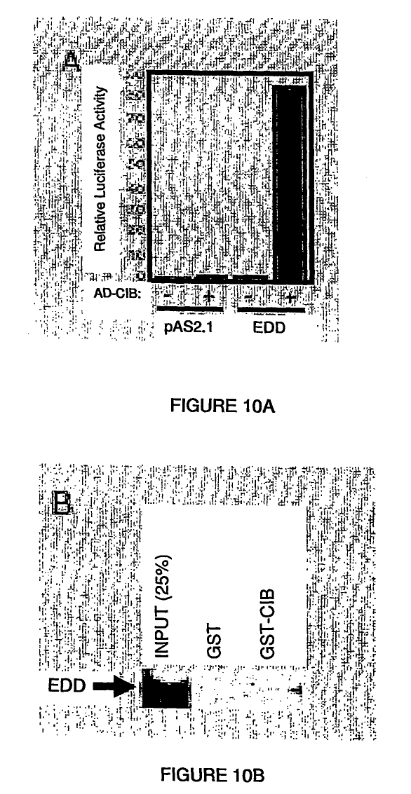

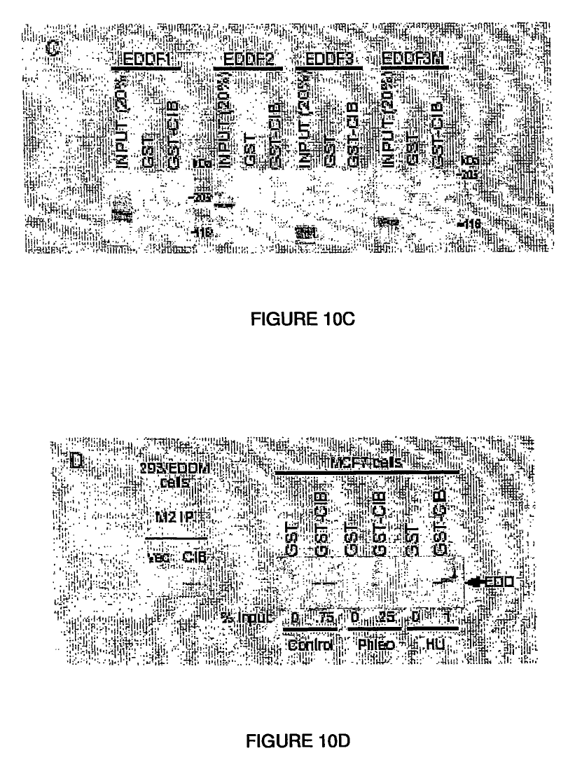

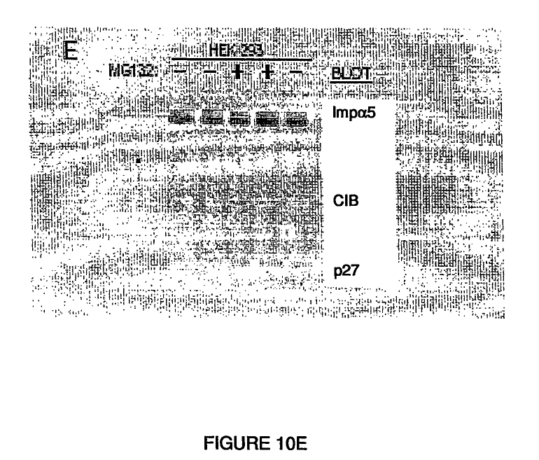

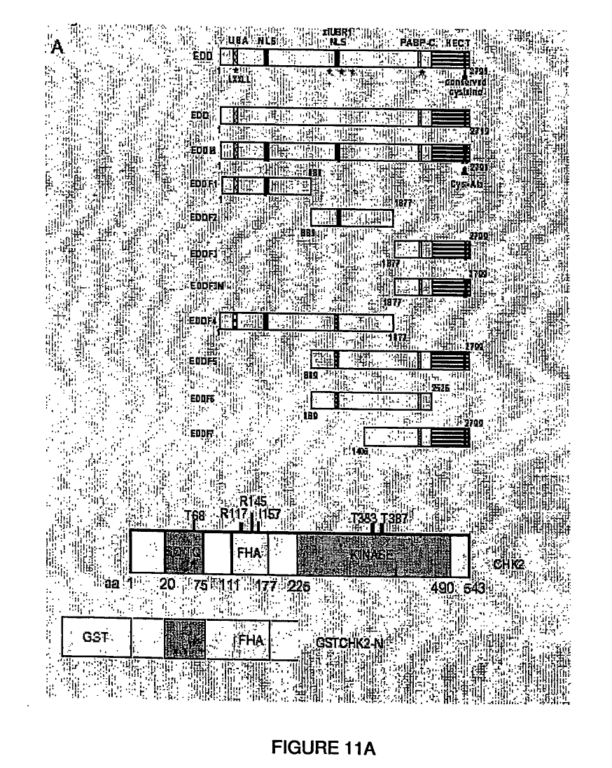

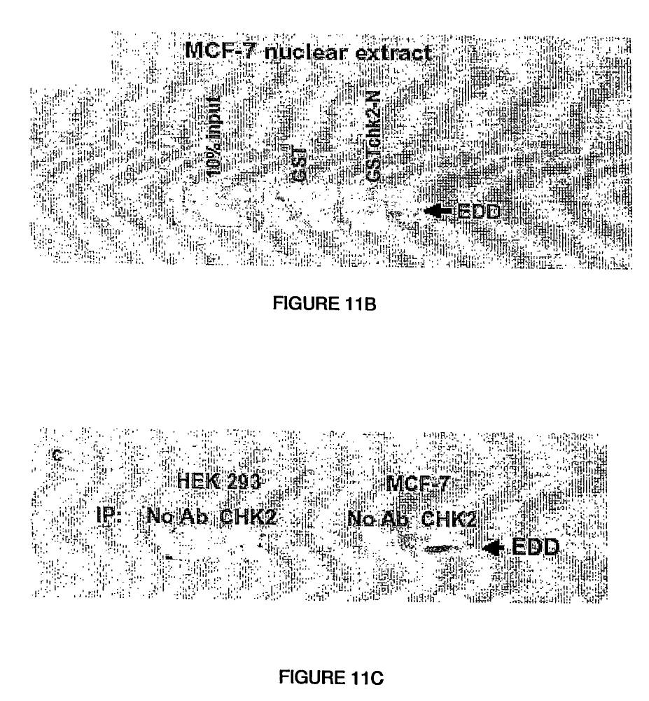

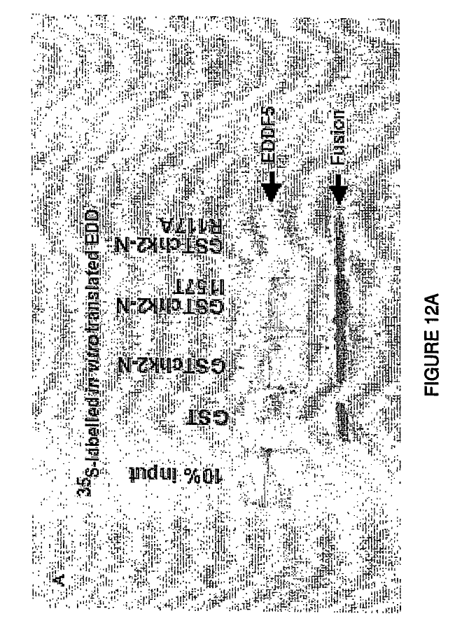

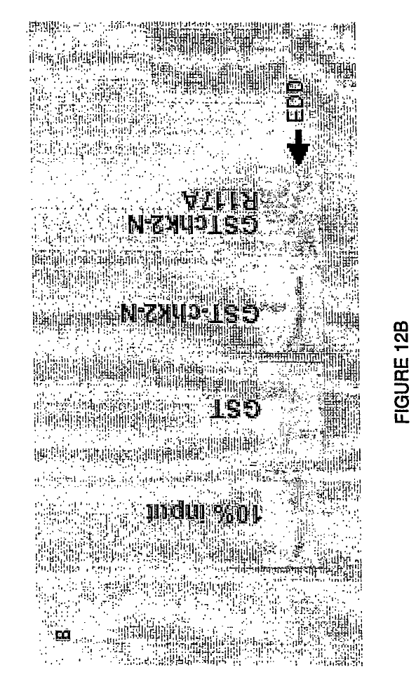

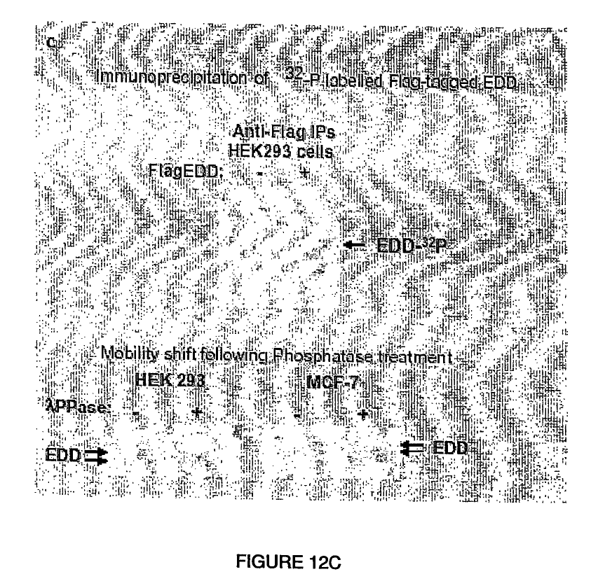

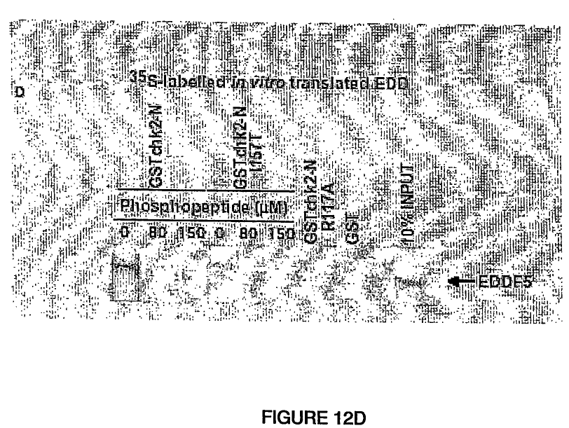

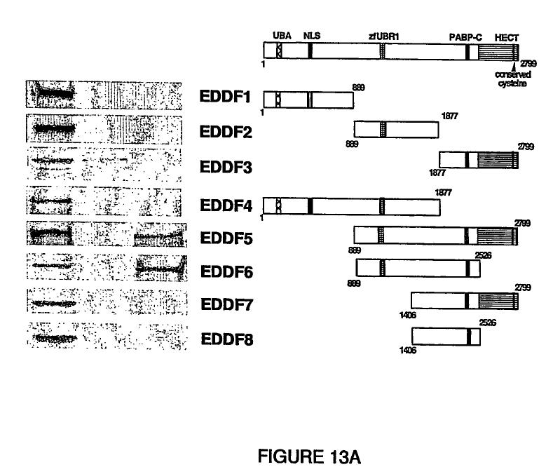

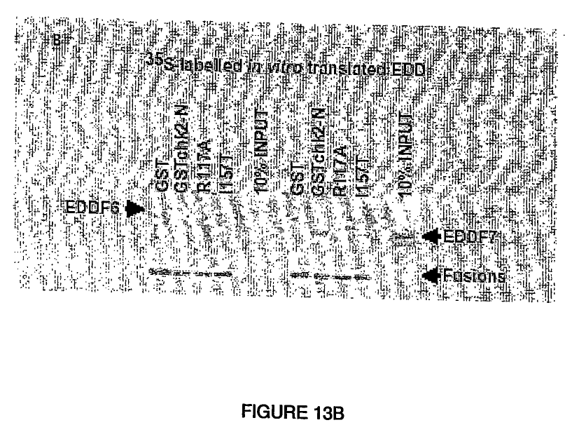

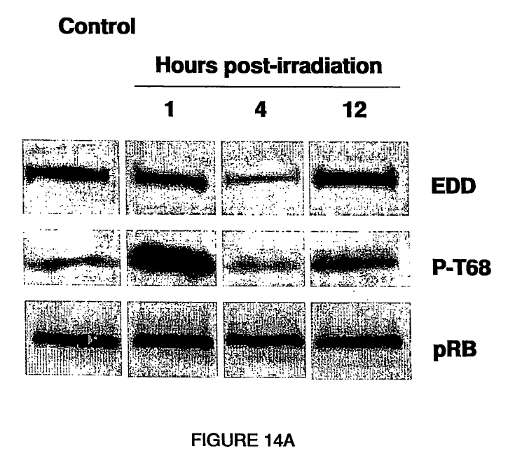

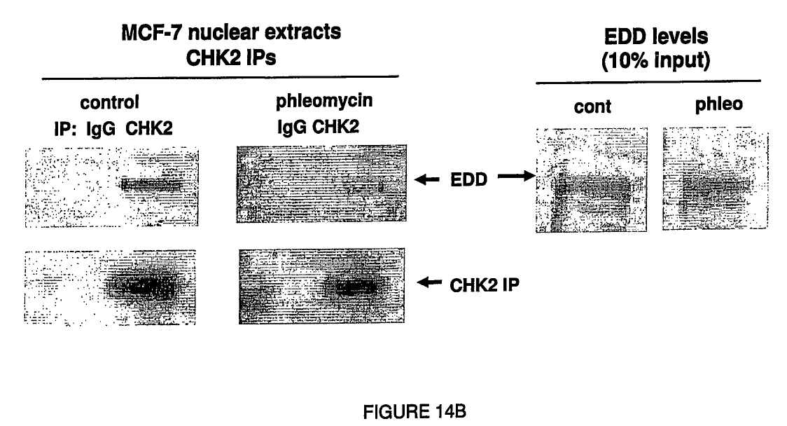

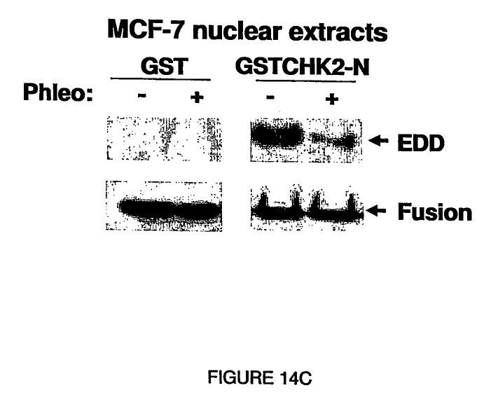

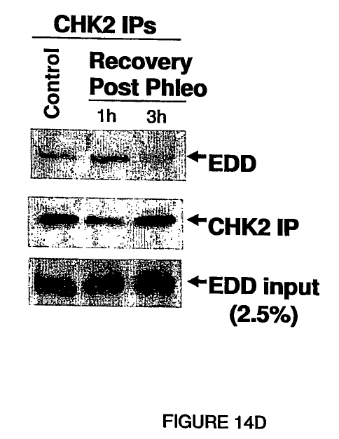

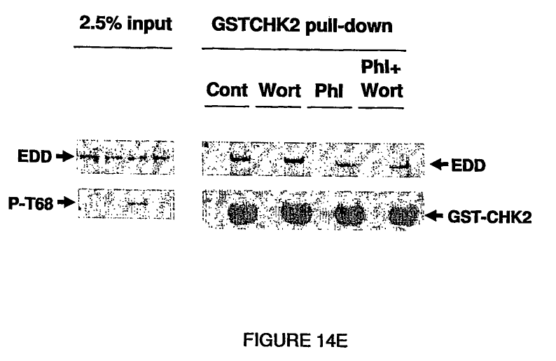

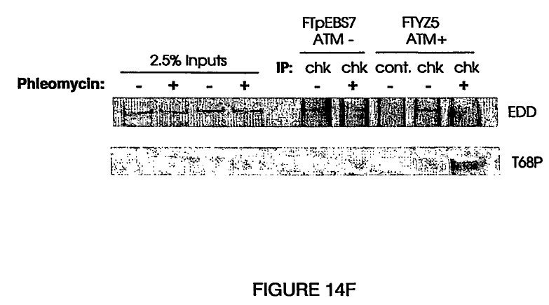

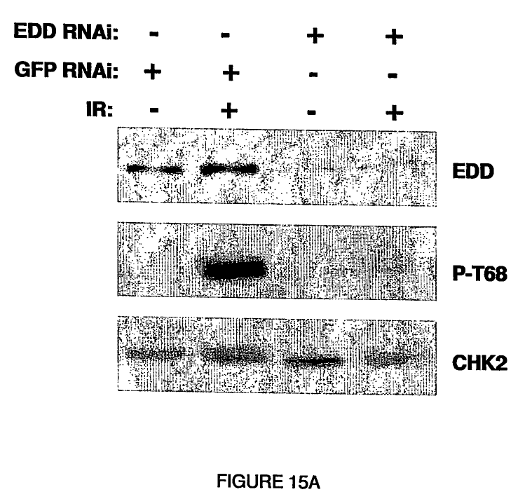

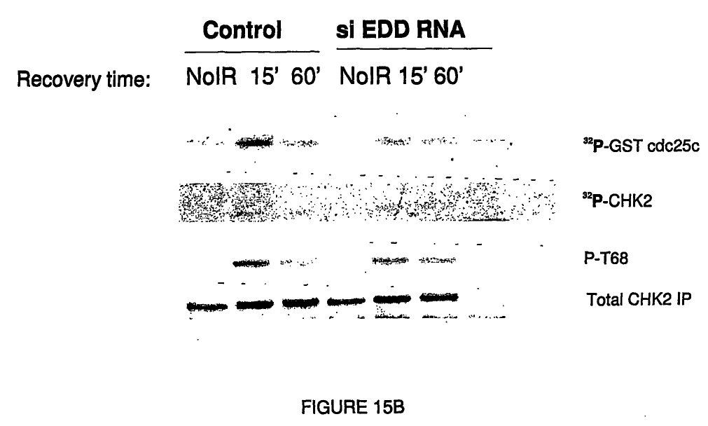

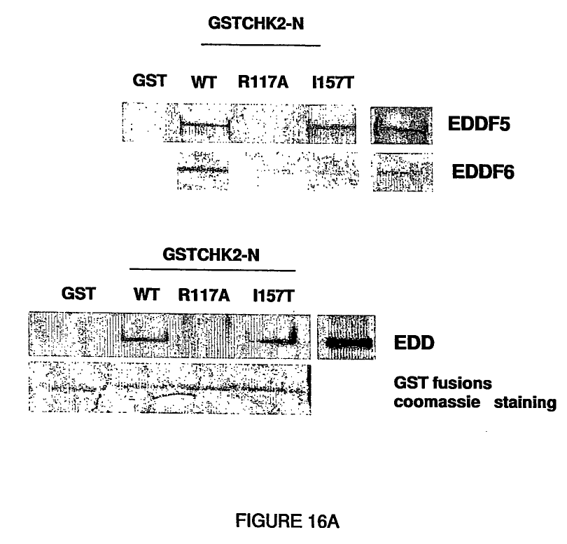

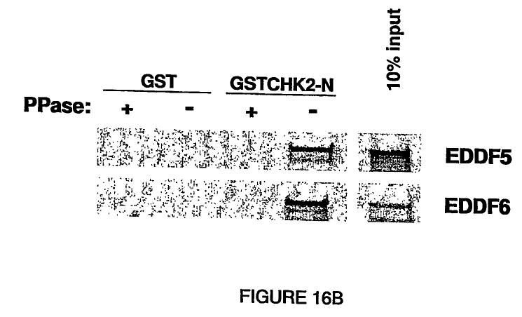

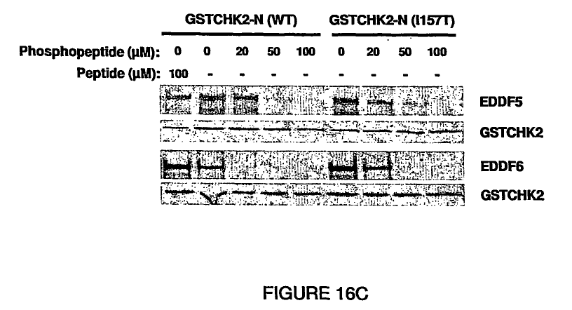

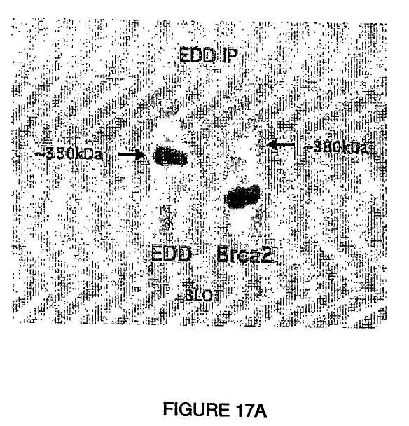

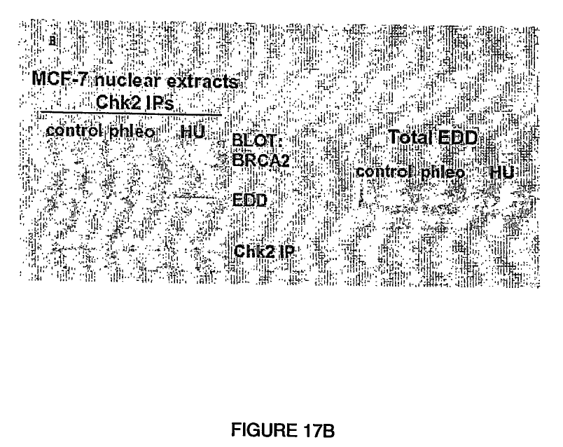

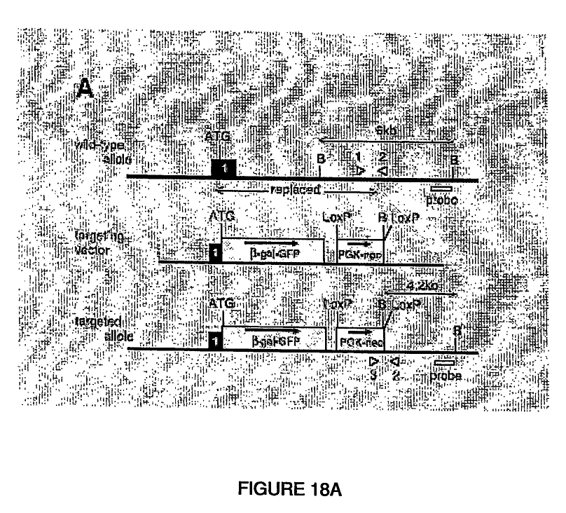

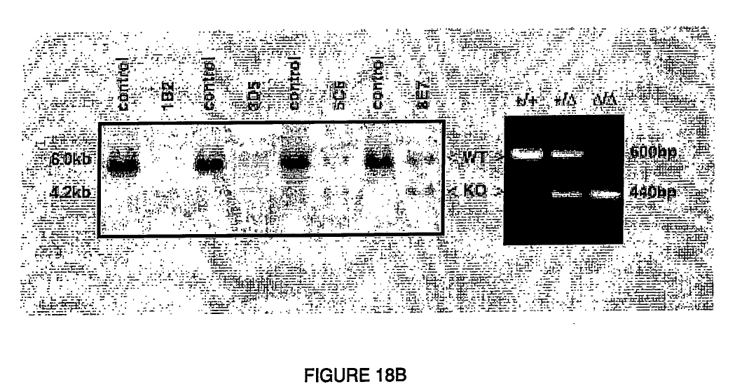

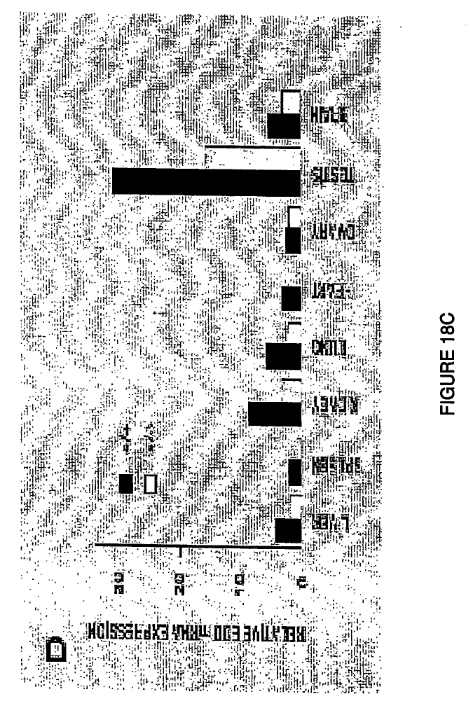

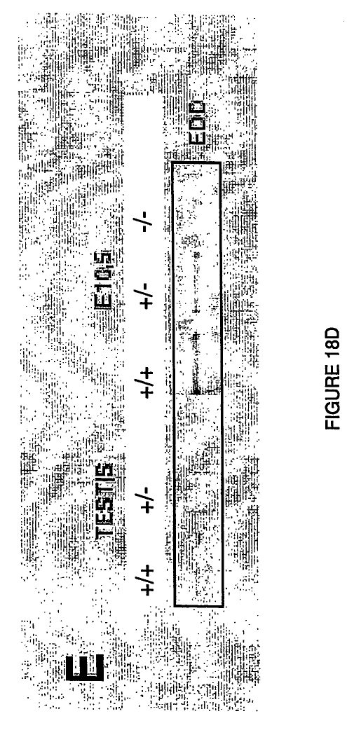

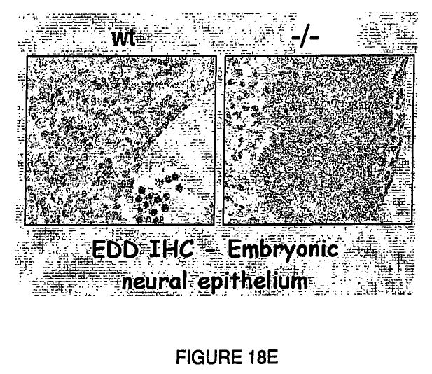

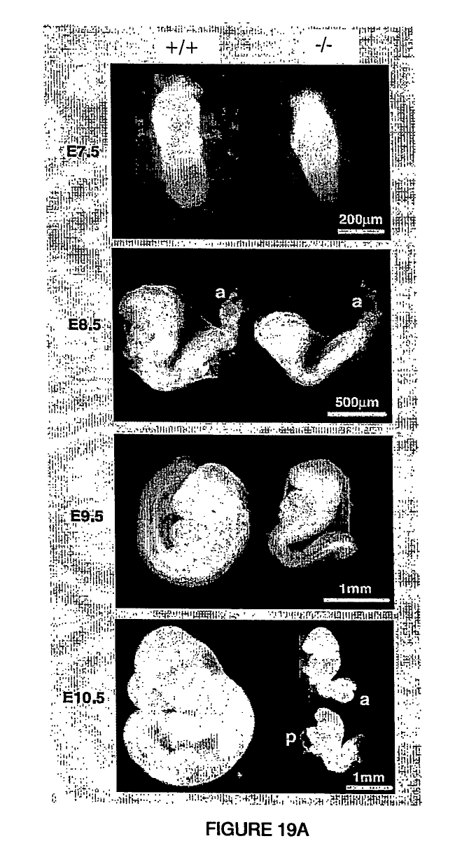

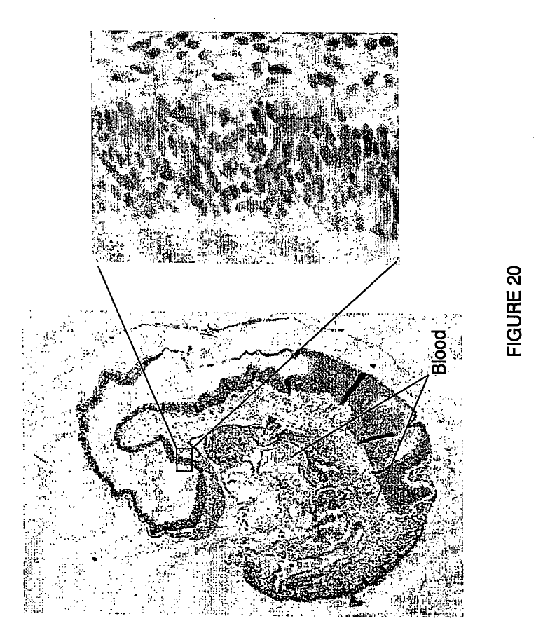

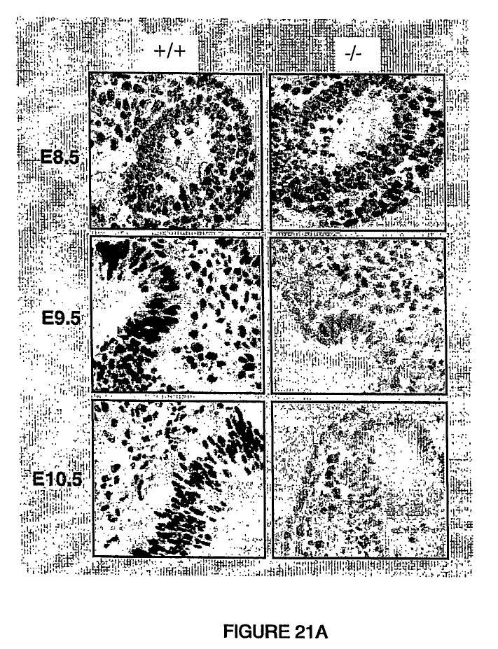

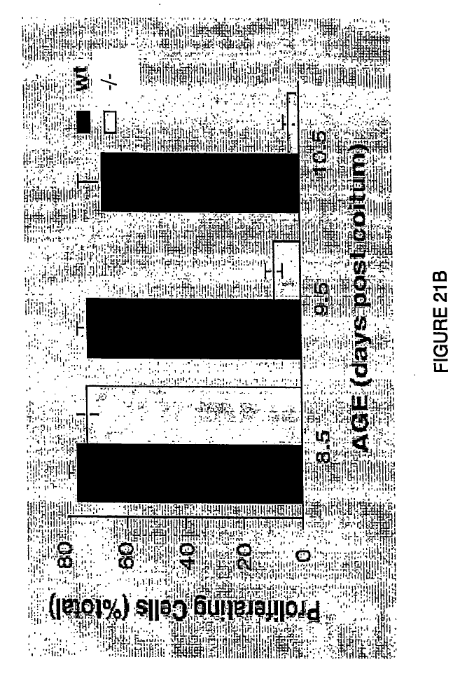

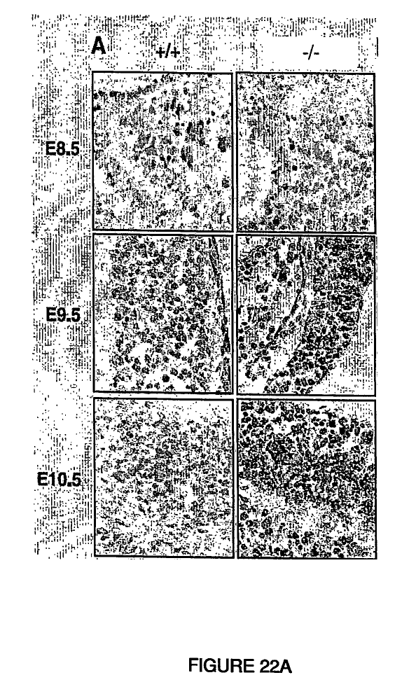

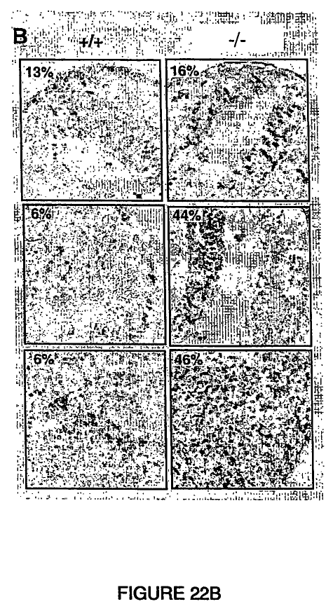

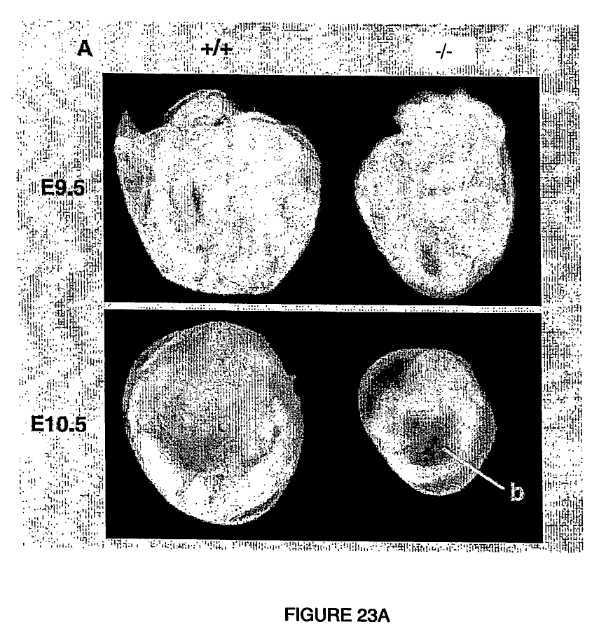

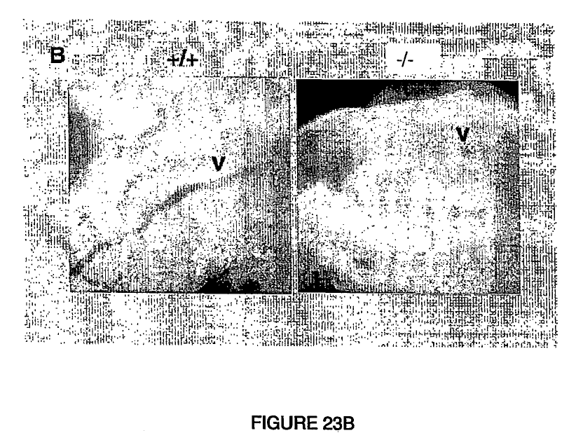

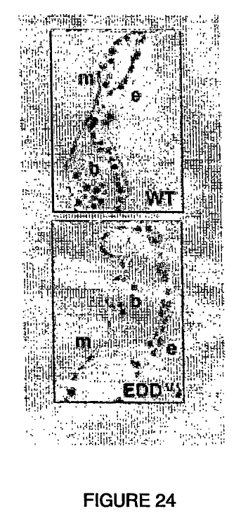

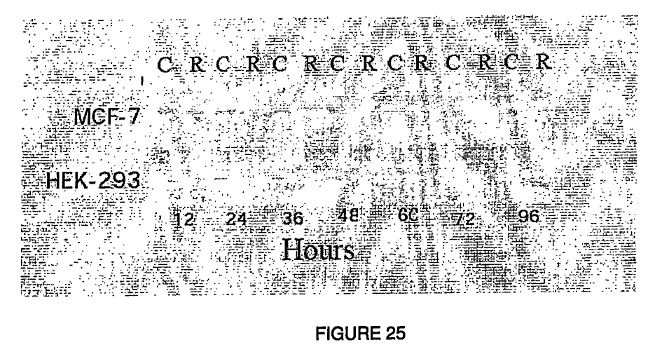

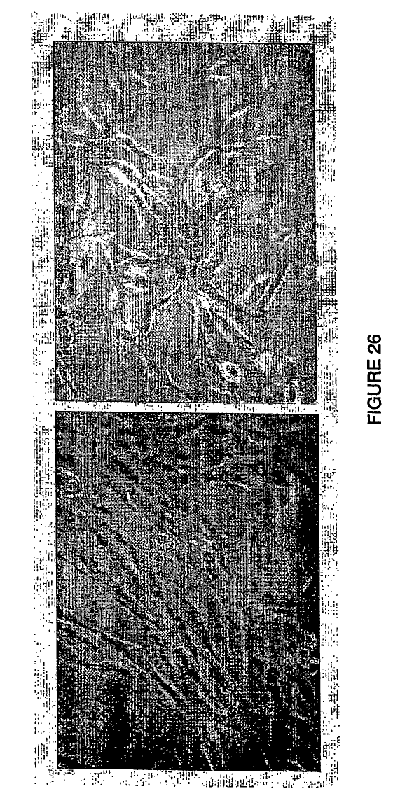

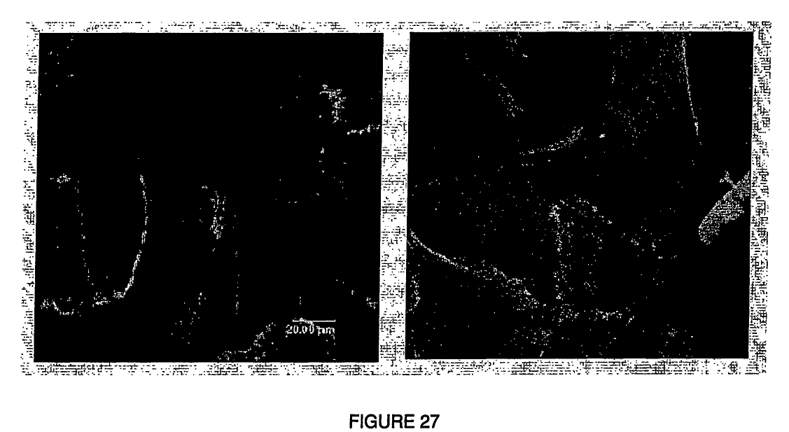

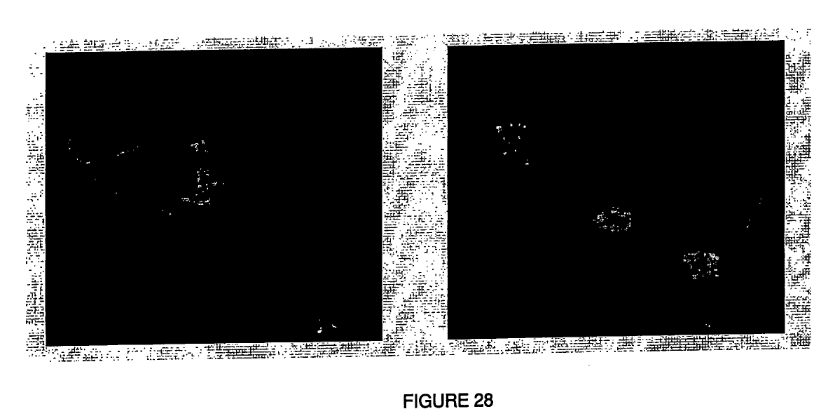

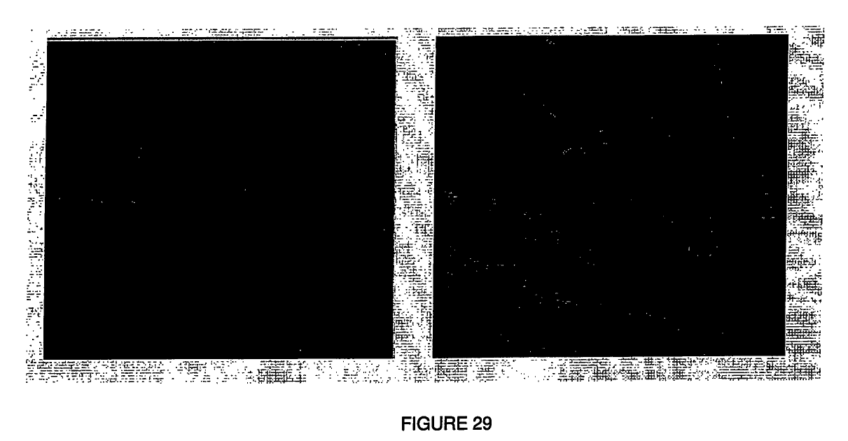

EDD mRNA expression levels were determined by quantitative RT-PCR in 41 breast cancers, and in matched normal breast tissue controls for 14 of these cases (FIG. 3A). Although the majority of cancers expressed EDD mRNA within the normal range, a significant number 11/41 (27%) had higher expression. Elevated expression was even more apparent when cancers were compared to their matched normal tissue controls, such that 6/14 (43%) cancers had >4-fold increase in EDD expression, including one cancer with a 159-fold increase (Inset FIG. 3A). Samples were also analysed for expression of several control genes, which controlled for epithelial content (CK18), cellular metabolism (GAPDH), general transcriptional activity (IF2B) or proliferation rate (MCM3) of the tumors analysed. EDD expression was not significantly altered when normalised to the expression of these genes.

1.7 EDD Protein Expression in Breast and Ovarian Cancers.

EDD protein levels were determined by immunohistochemistry in 9 specimens of normal breast tissue, 46 breast carcinomas and 94 serous ovarian carcinomas. Positive controls of wild-type EDD embryonic mouse neural tissue (FIG. 4B) and WT-30 cells (not shown) demonstrated intense nuclear staining while no expression was seen in EDD null embryonic mouse neural tissue (FIG. 4A). Of 9 normal breast samples, 4 had no detectable expression and 5 had low level expression of EDD (FIG. 4C). Of 46 breast carcinomas, all expressed EDD and demonstrated either low intensity (37%) or high intensity (63%) nuclear staining (FIG. 4D). Among the 94 serous ovarian carcinomas, 2% failed to express EDD while 59% had low (FIG. 4E) and 39% had high expression (FIG. 4F).

Univariate analysis of clinicopathological parameters of 165 patients with serous ovarian cancer shows that disease stage and cancer grade, overall patient health (performance status) and residual disease following surgical debulking all predict final patient outcome (ie whether or not a subject with serous ovarian cancer will survive) (Table 3). Analysis also showed that disease stage, presence of residual disease and EDD overexpression were predictive of disease relapse. Accordingly, EDD expression are clearly prognostic/diagnostic of relapse of serous ovarian cancer.

Multivariate analysis of clinicopathological parameters of 165 patients with serous ovarian cancer shows that disease stage and residual disease following surgical debulking are predictive of a poor outcome (ie patient death or cancer relapse) (Table 4). Analysis also showed that disease stage, presence of residual disease and EDD overexpression were predictive of disease relapse. Accordingly, EDD expression is clearly an independent prognostic/diagnostic marker of relapse of serous ovarian cancer.

1.8 EDD, p53R2 and CA II mRNA Expression and Genomic CopV Number in Breast Cancer Cell Lines

Expression profiling of 18 breast cancer cell lines with known DNA copy number showed a strong trend for EDD to be overexpressed at the mRNA level when EDD was amplified at the DNA level (FIG. 3B). Almost all cell lines where EDD was overexpressed had amplified EDD at the genomic level (⅞), whereas EDD was rarely overexpressed when the gene copy number was two or less. This was also true for the expression of p53 ribonucleotide reductase (p53R2), the gene adjacent to EDD at chromosome position 8q23.1. All cell lines where p53R2 was overexpressed had increased gene copy number (FIG. 3B). The relative expression of EDD and p53R2 was well correlated (R2=0.62) suggesting a similar mechanism of overexpression for both genes. In contrast, the expression of carbonic anhydrase II (CA II), located at 8q22, showed no relationship with DNA copy number (data not shown), with 15/18 cell lines having less than 10% of the expression levels of CA II mRNA of 184 normal breast epithelial cells.

1.9 Mutational Analysis of EDD in Cell Lines and Tumors

To assess the frequency of mutations in the EDD gene in cancer, the complete coding region of the EDD mRNA (8397 nucleotides) was sequenced using cDNA derived from 26 breast, ovarian and prostate cancer cell lines. Three normal breast epithelial cell lines were also sequenced. A list of the sequence variants identified is shown in Table 5. Only 2/25 cancer cell lines had variations in EDD mRNA that resulted in a change to the translated amino acid sequence. These putative missense mutations were confirmed in the genomic sequence. The amino acid changes were not in regions of the EDD protein having functional motifs and only the His>Asn change in the SK-Br-3 line alters amino acid polarity. In the absence of matched normal DNA from the individuals from which the cell lines were derived it is not possible to determine whether these represent true somatic mutations or rare polymorphisms. Few polymorphisms or silent substitutions were observed. Of the six conservative sequence variants, at least five are likely to be polymorphisms as they are either found in RNA derived from normal cell lines or tissues, or in multiple cell lines. A splice variant was also observed in all cell lines. The variant differs from the full length EDD mRNA by deletion of 18 bp of sequence from nt 884-901, removing the amino acid sequence VLLLPL. Although this motif is not located in any of the putative functional domains of EDD, removal of this sequence does have the potential to disrupt protein structure and might alter enzymatic function or localization of the protein.

SSCP analysis of twenty nine ovarian cancers displaying AI (FIG. 2), 37 breast cancers and 29 colon cancers confirmed the low frequency of mutation of the EDD gene, although only eight exons of EDD (covering approximately 13% of the coding sequence) were studied. These exons encode a nuclear localization signal (nt 1482-1598), a zinc finger motif (nt 3573-3812) and the majority of the HECT domain (nt 7602-8339) (Henderson et al, J. Biol. Chem. 277, 26468-26478, 2002). No mutations were found in the coding regions or splice junction sites of any cancer (data not shown).

1.10 Discussion

Microsatellite allelotyping shows that a narrow region of chromosome 8q that encompasses the locus for EDD (8q22.3) is a significant and specific area of chromosomal abnormality in ovarian cancer, hepatocellular carcinoma, squamous cell carcinoma of the tongue, breast cancer and metastatic melanoma. This was particularly evident in serous carcinoma of the ovary, where AI of the EDD locus occurred in approximately 70% of cancers. Overexpression of EDD mRNA was observed in a significant proportion of breast cancers and breast cancer cell lines, suggesting the possibility that EDD may be a target for amplification in this region of 8q.

Evidence that the region of 8q containing EDD is a specific focus of chromosomal abnormality is particularly clear for serous carcinoma of the ovary in which the frequency of AI at the EDD gene-specific microsatellite CEDD (73%) was almost twice that of the most telomeric microsatellite examined. Ovarian carcinomas commonly show a high level of chromosomal rearrangement in comparison with other tumor types. This is subtype specific with serous tumors displaying the greatest changes, endometrioid intermediate, and mucinous cancers the least (Pieretti et al, Int J. Cancer 64, 434-440, 1995), as was the case for AI at 8q in our study. In at least one study the invasive potential of the serous subtype has been correlated with changes to chromosome 8 (Diebold et al, ab. Invest 75, 473-485, 1996), although no correlation between clinical data (including grade and stage) and AI at 8q was found in our study. Although the very low rates of AI at EDD in benign and borderline ovarian tumors might indicate that this region is perturbed only at late stages in tumor progression, it is debatable whether such tumors are actually precursor lesions for malignant ovarian tumors.

Analysis of EDD expression clearly shows that EDD mRNA levels correlate with patient survival in all type of ovarian cancer studied. Furthermore, EDD protein levels correlate significantly with disease relapse. Accordingly, EDD expression levels (mRNA or protein) are clearly predictors of patient survival and disease relapse.

Despite the frequency of gross aberrations at the EDD locus and the common overexpression of the gene, EDD coding region mutations are apparently rare in cancer. Sequencing of the large EDD mRNA (8397 nt) revealed only 2/25 cancer cell lines with single nucleotide changes that result in amino acid changes. These alterations might represent rare polymorphisms rather than mutations as none of the changes were clearly disruptive and occurred in regions of the protein without any predicted functional significance. A splicing variant was found but this was present along with the unspliced transcript in every normal and cancer cell type examined. Similarly, SSCP analysis of a limited number of exons covering 13% of the coding region, and which code for putative functional domains, found no evidence of mutations in a series of breast, ovarian and colon cancers. This sequence conservation may point to a critical requirement for this gene in cell function, a conclusion reinforced by the hyd knockout lethality (Mansfield et al, Dev. Biol. 165, 507-526, 1994) and by our recent finding that targeted deletion of EDD in mice results in embryonic lethality (unpublished data).

### EXAMPLE 2

### Nuclear Function of EDD HECT Ligase

2.1 Experimental Procedures

Plasmid constructs

EDD cDNAs used for in vitro translation, transfection and yeast two-hybrid screening, are shown in FIG. 5A. cDNAs encoded the full length protein EDD (aa 1-2799), the N-terminal domain EDDF1 (aa 1-889), the central domain EDDF2 (aa 889-1877), the carboxy domain EDDF3 (aa 1877-2799), the N-terminal plus central domains EDDF4 (aa 1-1877) and the central plus C-terminal domains EDDF5 (aa 889-2799). EDDM, EDDF3M and EDDF5M contain a mutation (Cys2768 to Ala) at the active site cysteine necessary for E3 ligase activity in HECT proteins. For mapping of the EDD N-terminus, restriction fragment cloning was used to generate in vitro translation constructs expressing EDD aa 1-577 (EDDF1a), 578-889 (EDDF1b), 1-419 (EDDF1c), and 420-889 (EDDF1d) (FIG. 5A). For yeast two-hybrid screening, EDD cDNA fragments used as baits were cloned from pBluescript-EDD (Callaghan et al, Oncogene 17, 3479-3491, 1998) in frame with the Gal4 DNA binding domain (DBD) of the pAS2.1 vector (Clontech Laboratories, Palo Alto, Calif., USA). For in vitro translation, EDD-derived cDNAs were transcribed from pBluescript (Amersham Pharmacia Biotech, UK), pSG5 (Stratagene, La Jolla, Calif., USA) or pRcCMV (Invitrogen, Gronigen, Netherlands) vectors. For EDD protein expression in mammalian cells, constructs in pRcCMV have been previously described (Callaghan et al, Oncogene 17, 3479-3491, 1998) and additional constructs for expression of FLAG epitope tagged EDD were generated in the pSG5 vector. A GFP reporter vector (pGFP20, Dr S. Aota, Osaka, Japan) was co-transfected to monitor transfection efficiency. A bacterial plasmid expressing GST fused to amino acids 263-538 of human importin α5 (NPI-1) was obtained from Peter Palese (Mount Sinai School of Medicine, NY, USA). A GST fusion of mouse importin β1 (PTAC97), was expressed and purified as described previously (Hubner et al, 1997). Full-length CIB was cloned from the pACT2 vector into the pGEX2T vector for GST-CIB fusion protein expression in bacteria and into the pCMVTag2C vector for mammalian expression of FLAG-tagged protein. For expression of GFP-tagged EDD in mammalian cells, full length EDD was cloned into pEGFP-C1 (for N-terminal EGFP tag) and pEGFP-N1 (for C-terminal EGFP tag, Clontech Laboratories). For transient expression of progesterone receptor, phPR1 vector encoding human PR B was obtained from P. Chambon (INSERM, France). A PRE-luciferase reporter vector (pMSGIuc) was constructed by insertion of a MMTV-LTR promoter in the pGL3-Basic vector (Promega Corp.). The phPR1 vector was used to clone the PR(AB) (aa 1-546) and PR(CDE) (aa 456-933) regions into pGEX4T2 for GST fusion protein expression. For transient expression of vitamin D receptor pCMV-VDR along with pOS2-luc reporter vector were obtained from G. Leong (Garvan Institute, Australia). Estrogen receptor was expressed from pCMV-ER (V. C. Jordan, Northwestern University Medical School, Chicago, USA) and pERE-TK-GL3 reporter vector was obtained from M. Parker (ICRF, UK). For in vitro translation, SRC-1 was cut from pCR3.1-SRClA (B. O'Malley, Baylor College, Texas) and cloned into pBluescript.

Yeast Two-Hybrid Assay for EDD Interacting Proteins

The cDNAs for full length EDD mutant and carboxy domain mutants (EDDM and EDD5M) were screened against a human placenta cDNA library in the pACT2 vector by the yeast two-hybrid method (Matchmaker 2, Clontech, Palo Alto, Calif., USA). Stable transformants of Saccharomyces cerevisiae strain Y190 expressing EDD fusion protein were transformed with the library using the lithium acetate method and 2-3×10⁶ primary transformants selected on his-leu-trp-plates. Following a second round of selection on the same medium, colonies were assayed for β.galactosidase activity using a filter-based assay. Interacting plasmids that were positive for β.galactosidase only in the presence of the EDD bait plasmids were transformed into E. coli DH5α cells for further analysis. Manual sequencing was carried out using ³³P end-labelled primer in conjunction with the fmol Cycle sequencing kit (Promega Corp., Madison, Wis., USA). Sequences were analysed by Blast searches of the Genbank and EMBL databases and predicted proteins analysed for motifs using the ISREC Profile Scan Server (www.isrec.isb-sib.ch).

For semi-quantitation of protein interactions, CG1945 yeast cells containing pAS2.1-EDD constructs were mated with Y187 yeast cells harbouring pACT2-derived plasmids. Diploids were selected on leu-trp-plates and used to inoculate cultures which were grown to saturation, diluted 1:10 and grown for 16 h. Yeast cells were harvested for protein and β.galactosidase activities were determined in a liquid chemiluminescence assay (Tropix Galacto-Light System, Applied Biosystems, CA, USA).

Recombinant Protein Binding Assays

GST-tagged fusion proteins were prepared from E. coli strain BL21 according to established protocols (Pharmacia Protocol Handbook). Soluble fusion proteins were bound to glutathione agarose and quantitated via Coomassie blue staining against protein standards. 35S-labelled EDD protein and mutants or SRC-1 were synthesised in a coupled in vitro transcription/translation system (TNT Quick, Promega) and 10-20 μl reaction mixture was diluted in 1% Triton X-100 lysis buffer (Callaghan et al, Oncogene 17, 3479-3491, 1998) and incubated with 5 μg of GST, GST-importin α5, GST-PR(CDE) or GST-CIB coupled to glutathione agarose beads at 4° C. for 2 h. Beads were collected by centrifugation, washed extensively in lysis buffer and resuspended in SDS-PAGE sample buffer. After boiling, bound protein was visualised following SDS-PAGE and autoradiography.

Cell Culture and Transient Transfection

HEK 293 and T-47D were maintained as previously described (Callaghan et al, Oncogene 17, 3479-3491, 1998). MCF-7 cells were maintained in RPMI medium (Life Biotechnologies) containing 10% serum in 5% CO2. For overexpression by transient transfection, 3×10⁶ HEK 293 cells were plated in HMEM containing 10% serum in 15 cm petri dishes. The following day pRcCMV-EDD (10 μg) was added to the cells along with 30 μl Fugene reagent (Roche Diagnostics, Castle Hill, NSW, Australia).

Localisation of GFP-Tagged and Endogenous EDD Protein

HEK 293, CHO, MCF-7 or T-47D cells were seeded in 6-well plates at 1-2×10⁵ cells/well. Cells were transfected with 2 μg pEGFP-EDD or empty vector DNA and the following day split to chamber slides for 24-48 h. Slides were washed in PBS, fixed in 3.7% paraformaldehyde, washed in PBS and mounted in 90% glycerol. GFP was visualised by fluorescence microscopy. For immunostaining, HEK 293 cells or EDD transfected HEK 293 cells (WT30) were embedded in paraffin. Sections were de-waxed and rehydrated before unmasking in EDTA/Citrate buffer and then stained with goat anti-EDD antibody N19 (Santa Cruz, Calif., USA). EDD signal was detected using DAKO LSAB Plus Link and Label (DAKO Corporation, CA, USA) with liquid 3,3′-diaminobenzidine Plus (DAKO Corporation, CA, USA) as substrate. Counterstaining was performed with hematoxylin.

Protein Interactions in Cell Lysates

For extracts of total cellular protein, cells were harvested in 1% Triton X-100 lysis buffer as previously described (Callaghan et al, Oncogene 17, 3479-3491, 1998). Extraction of nuclear proteins and cytoplasmic s100 fractions from T-47D and MCF-7 cells was carried out according to published methods (Dignam et al, Nucleic Acids Res. 11, 1475-1489, 1983).

For GST fusion protein pull-down of endogenous or recombinant EDD from cell lysates, 0.5 to 1 mg total protein was incubated with 5 μg GST or GST fusion protein bound to glutathione beads for 1-2 h at 4° C. Beads were washed extensively in 1% Triton X-100 lysis buffer and bound proteins resolved by SDS-PAGE and detected by immunoblotting with EDD antisera (Callaghan et al, Oncogene 17, 3479-3491, 1998). For immunoprecipitation, 10 μl importin α5 antisera (Peter Palese, Mount Sinai School of Medicine) was incubated with 0.5 to 1 mg cell lysate (4° C. for 1 h). Antibody conjugates were captured on protein A sepharose beads (4° C. for 1 h) and washed extensively in lysis buffer. Bound proteins were resolved by SDS-PAGE followed by immunoblotting with EDD antisera.

Stable HEK 293 cells overexpressing EDDM were transfected with pCMVTag2C-CIB or empty vector using Fugene 6 reagent (Roche) for 24 h. Following 6 h incubation in the presence of MG132, cells were harvested and lysates prepared and one mg total protein incubated with anti-FLAG antibody M2 coupled to Sepharose (Sigma Chemical Co., St Louis, USA) for 2 h at 4° C. Beads were recovered by centrifugation and washed extensively in lysis buffer. Western blotting for EDD has been described (Callaghan et al, Oncogene 17, 3479-3491, 1998).

Nuclear Receptor Transactivation Assays

HEK 293 or COS7 cells were plated in 6-well plates (2×10⁵ cells/well) and the medium changed to 2% charcoal-stripped FCS the following day. Transfection was carried out using 3-4 μl Fugene 6 Reagent (Roche) with 1-2 μg DNA comprised of 90 ng receptor expression vector, 450 ng luciferase reporter vector and 1.2 μg EDD, EDDM or SRC-1 cDNAs in either pRcCMV or pSG5, or empty vector, and 200 ng GFP expression vector pGFP20. The following day cells were split into 96-well plates (7×10³ cells/well) or 6-well plates (1.4×10⁵ cells/well), and drugged 24 h later. After a further 24 h cells in 96-well plates were assayed for luciferase activity (Luclite reagent, Packard Bioscience, Meriden, Conn., USA) and cell number (Wst-1 reagent, Roche). In some experiments cell number was monitored using the CellTiter96® Proliferation Assay (Promega Corp., Madison, USA). Cells in 6-well plates were analysed for GFP expression by fluorescence microscopy to determine transfection efficiency and also used for preparation of protein lysates so that protein expression levels of various constructs could be compared. In experiments where there was significant variation in cell number or GFP expression these parameters were used to normalise luciferase activity. In some experiments pRSV-β-gal or pRL-TK (Promega Corp., Madison, USA) vectors were transfected in place of pGFP20 and transfection efficiency monitored by assaying for β-galactosidase or Renilla luciferase activity respectively.

Proteasome Inhibition Experiments

For proteasome inhibition experiments, HEK 293 cells were plated at 3×10⁶ cells/well of a 6-well dish in minimal essential medium with Hanks' salts (HMEM) supplemented with 10% fetal bovine serum (Life Technologies, Gaithersburg, Md., USA). After 48 h, medium was replaced with medium containing 20 μM MG132 (Calbiochem, CA, USA) or DMSO vehicle for 2-6 h. A monoclonal antibody for western blotting against CIB was kindly provided by U. P. Naik (University of North Carolina, NC, USA).

Treatment of Cells with DNA Damaging Agents

MCF-7 cells were incubated in RPMI containing 0.5% foetal bovine serum for 18 h before addition of phleomycin (Cayla, France) at 100 μg/ml, hydroxyurea (Calbiochem, CA, USA) at 2 mM or PBS vehicle, for 6 h. Cells were harvested for total protein or nuclear protein extracts as described above.

2.2 Domain structure of EDD

Previous analysis of the EDD sequence showed the presence of a carboxy-terminal HECT domain, identifying EDD as a member of the HECT family of ubiquitin protein ligases (Callaghan et al, Oncogene 17, 3479-3491, 1998; and Huibregtse et al, 1995) (FIG. 5A). Further examination of the central domain of EDD that is also highly conserved with HYD revealed a stretch of 68 amino acids (aa 1177-1245) that is 95% and 100% conserved with HYD and mouse EDD, respectively (FIG. 5B) and which shows a high degree of alignment with calossin (pushover), a calmodulin-binding protein important for neurotransmission and male fertility in Drosophila (Richards et al, Genetics 142, 1215-1223, 1996; Xu et al, J. Biol. Chem. 273, 31297-31307, 1998). Contained within this region is a cysteine/histidine-rich putative zinc finger domain, zf-UBR1 (Pfam PF02207, (Bateman et al, Nucleic Acids Res 28, 263-266, 2000)), originally identified in the N-end rule E3 ubiquitin ligase UBR1p/N-recognin from a range of species (Bartel et al, EMBO J. 9, 3179-3189, 1990; Varshavsky, Cell 69, 725-735, 1992; Kwon et al, Proc. Natl. Acad. Sci. USA 95, 7898-7903, 1998). The consensus sequence CX₁₂₋₁₆CX₂CX₈₋₁₀CX₂CX₄₋₅HX₂HX₁₁₋₁₄CXCX₄₋₁₄C is reminiscent of the more common RING domain, which is also found in a distinct region of UBR1p. Both types of zinc-binding domains are proposed to have roles in protein-protein interaction, with the RING domain having an established role in ubiquitinylation (Freemont, Curr. Biol. 10, 84-87, 2000; Lorick et al, Proc. Natl. Acad. Sci. USA 96, 11364-11369, 1999). This central region of EDD also contains a potential bipartite nuclear localisation sequence (NLS), (KLKRTSPTAYCDCWEKCKCK, aa 1222-1241; SEQ ID NO: 43) while another putative NLS resides in the N-terminal region (RKKMLEKARAKNKKPK, aa 502-517; SEQ ID NO: 44) upstream of a potential SV40 large T antigen-like NLS (PYKRRR, aa 630-635; SEQ ID NO: 45) (Dingwall et al, Trends Biochem Sci 16, 478-781, 1991). Also within the N-terminal region of EDD is another region conserved with HYD, designated as a “UBA domain” (aa 188-225), which may form a protein-protein interaction interface (Hofmann et al, TIBS 21, 172-173, 1996). Amino terminal to the HECT domain (aa 2391-2455) lies still another region that may mediate protein interactions. This 60 amino acid stretch shows 50% homology to a region within the carboxy terminus of polyA binding proteins (PABP-C) from a range of species (Callaghan et al, Oncogene 17, 3479-3491, 1998; Kozlov et al, Proc. Natl. Acad. Sci. USA 98, 4409-4413, 2001). The X-ray structure of this domain in both PABP and EDD has recently been determined and forms a protein interaction interface consisting of four alpha helices (Kozlov et al, Proc. Natl. Acad. Sci. USA 98, 4409-4413, 2001; Deo et al, Proc. Natl. Acad. Sci. USA 98, 4414-4419, 2001).

2.3 Interaction Between EDD and Importin α-5

Yeast two-hybrid approaches were used to identify interacting proteins that may be ubiquitinylation targets of EDD or other associating proteins with a role in EDD function. First, yeast two-hybrid screening of a human placental cDNA expression library was performed against baits encoding full length EDD or fragments containing one or more potential interaction domains of EDD (see FIG. 5A). Screening with full length EDDM (C2768A mutant) identified two independent cDNAs encoding the nuclear import protein importin α5 (NPI-1, (O'Neill et al, Virology 206, 116-125, 1995; Kohler et al, Mol. Cell Biol. 19, 7782-7791, 1999)), one full-length and the other encoding amino acid 229 to the carboxy terminus (amino acid 538). Importin a has a specific role in nuclear import, by recognising NLSs, implying both that one or more of the potential NLSs within EDD are indeed functional, and that EDD may have a role in the nucleus, with importin α involved in transporting EDD from the cytoplasm to the nucleus.

2.4 lmportin α-5 Interacts with a Region of EDD Containing Nuclear Localisation Signals Results

A strong interaction was found between NPI-1/importin α5 and full length EDD, with full length importin α5 interacting more strongly than the amino-truncated protein isolated by two hybrid screening (FIG. 6A). This difference might be explained if EDD, like other proteins that contain basic NLSs, is recognised by the armadillo repeats of importin α: only four of seven such repeats are present in the truncated importin α5 clone. Pull-down experiments showed that a GST importin α5 fusion protein encoding amino acids 263-538 was able to bind to in vitro translated EDD and mutants encoding the N-terminal one-third of EDD but not to the central or carboxy terminal regions of EDD (FIG. 6B, 6C). This suggested that the putative NLS in the central region of EDD was not functional in nuclear import. GST-importin α5 also interacted with endogenous EDD (T47D cells) and recombinant EDD expressed in HEK 293 cells (FIG. 6D), precipitating a considerable proportion of the available EDD protein. Further, anti-importin α5 antisera also immunoprecipitated EDD protein from these lysates showing that EDD and importin α5 interact in vivo (FIG. 6D).

The amino terminal one-third of the EDD protein contains two potential basic NLSs, one bipartite and one simple. To determine the relative contributions of these motifs to importin α binding, a set of constructs for in vitro translation were made that contained one, both or neither NLS. GST-importin α interacted with each NLS to some degree and no interaction was seen in the absence of both signals (FIG. 6E). We therefore conclude that both N-terminal signals are required for full binding potential.

We expected that EDD might be in a nuclear import complex with importins α and β so binding to the nuclear import partner importin β/p97 was also tested (FIG. 6F). GST-importin β bound to in vitro translated EDD and the amino two-thirds or one-third of the EDD protein, resulting in pull-down of approximately 5% of the available EDD protein. As the extracts used for in vitro translation contain endogenous importin α, binding of EDD and importin β is most likely mediated by the importin α/β heterodimer. Yeast two-hybrid analysis also indicated EDD interaction with both importin α1 (Rch1) and importin α3 (Qip1), although the interaction between EDD and importin α5 was markedly stronger (data not shown). Overall, the interaction between EDD and several importin α isoforms and importin β, point to a role for EDD within the nucleus.

2.5 EDD is a Nuclear Protein

To determine the cellular localisation of EDD, mammalian expression vectors were made for EDD fused to the N or C terminus of green fluorescent protein (GFP). Transfection of HEK 293 cells or MCF-7 breast cancer cells with the N-terminal EDD-GFP fusion showed that fluorescence was restricted to the nucleus (FIG. 7A). Identical results were obtained with the C-terminal fusion (not shown). In contrast, when cells from either line were transfected with pEGFP vector only, a 30 diffuse pattern of staining throughout the whole cell was observed. Nuclear localisation was confirmed when EDD-specific antibodies were used to stain sections of HEK 293 cells which endogenously express EDD or WT30 derivative of HEK 293 cells, which overexpress EDD (FIG. 7B). The same pattern of staining was seen for a second EDD-specific antibody (not shown).

2.6 EDD Interacts with Progesterone Receptor B

Previous studies have demonstrated that the HECT-domain protein E6-AP interacts directly with PR-B through a region containing LXXLL motifs (Nawaz et al, Mol. Cell Biol. 19, 1182-1189, 1999). These motifs which are present in other transcriptional coactivators are potentially involved in nuclear receptor interaction and coactivation (Heery et al, Nature 387, 733-736, 1997). Since the EDD protein is nuclear and contains five LXXLL domains (at amino acids 248, 1102, 1255, 1398 and 2428) we tested the ability of EDD to interact with PR-B and regulate its function. First we performed GST-PR fusion protein pull-downs of EDD or in vitro synthesised EDD fragments. The N-terminal AB region of PR contains a ligand-independent activation function 1 while the C-terminal CDE region of PR contains the hinge and DNA binding domains and a ligand-dependent activation function 2. The CDE region of PR, PR(CDE), interacted with endogenously expressed EDD from T-47D cells (FIG. 8A). This interaction was mapped using in vitro translated EDD protein fragments. A strong interaction was detected between the amino terminal region of EDD (EDDF1, aa 1-889) and the CDE region of PR, being greater than that seen for SRC-1 (FIG. 8B). In these in vitro assays, interactions between PR(CDE) and either SRC-1 or EDDF1 were not affected by the PR ligand ORG2058 (data not shown). No significant binding was observed between PR and other fragments of EDD (FIG. 8B and data not shown).

The N-terminal region of EDD contains one of the five LXXLL motifs so the interaction was mapped further to assess the involvement of this motif. EDD fragments EDDF1a-EDDF1d were tested for their ability to bind GST-PR(CDE). Although EDDF1a (aa 1-577) and EDDF1c (aa 1-419) contained the LXXLL motif, the strongest binding occurred between EDDF1b (aa 578-889) or EDDF1d (aa 420-889) and PR(CDE) (FIG. 8C), suggesting that binding is mediated by the region of EDD consisting of amino acids 420-889, which includes both NLSs but not the LXXLL motif. This also ruled out the involvement of the UBA domain in this interaction. Taken together, these data demonstrate an interaction between EDD and PR.

2.7 EDD Acts as a Transcrintional Coactivator for Nuclear Receptors

The nuclear localisation of EDD and the observed interaction between EDD and PR-B, together with evidence from separate studies that other HECT-domain proteins such as yeast Rsp5, its human homolog hRPF1 (Imhof et al, Mol. Cell. Biol. 16, 2594-2605, 1996), and E6-AP (Hubner et al, J. Biol. Chem. 272, 17137-17195, 1997) have coactivator activity for nuclear receptors, prompted an investigation of whether EDD could enhance transcriptional activation by PR-B. To this end, HEK 293 and COS7 cells, which lack endogenous PR, were transfected with a PR expression vector (pSG5/hPRB-1) and the progestin-responsive MMTV-luciferase reporter construct together with expression vectors for EDD, or SRC-1 as a positive control. EDD consistently increased progestin (ORG2058)-induced luciferase activity three- to five-fold above control levels in both lines, an effect comparable to that of SRC-1 (FIG. 9A). In the absence of added ORG2058, EDD and SRC-1 also slightly increased the basal activity of the luciferase MMTV-LTR promoter, an effect more apparent in the COS7 cell line. We next tested whether the observed transcriptional effect of EDD was due to the ubiquitin ligase activity of EDD. When the ligase-defective EDD mutant (EDDM) was transfected, a comparable coactivator activity was observed, suggesting that the coactivator activity of EDD is independent of its ubiquitin ligase activity (FIG. 9B).

Importantly no effect of EDD on PR transactivation is seen in the presence of the progestin antagonist RU486 (not shown), indicating specificity for ligand-bound receptor. Also, there was no effect of EDD on reporter gene activity in the absence of PR, indicating a specific effect on transactivation by PR (not shown). Transfection of increasing amounts of pRcCMV-EDD showed a clear dose response for effects on progestin-induced luciferase activity (FIG. 9C), and ORG2058 at all concentrations between 10 pM and 100 nM stimulated luciferase activity to a much greater extent when EDD was co-expressed (FIG. 9D). EDD co-expression resulted in a greatly enhanced response to low concentrations of progestins such that without EDD transfection, 10 nM ORG2058 gave a maximal response, whereas this was exceeded at a 100-fold lower concentration, 100 pM, with EDD overexpression.

These data reveal for the first time a cellular function for EDD as a nuclear receptor coactivator. Interestingly, EDD also enhanced transactivation by the vitamin D receptor (VDR) three-fold (FIG. 9E). However estrogen receptor (ER) activity was not enhanced by EDD, whereas in the same experiment SRG-1 acted as a coactivator (FIG. 9F), demonstrating that EDD discriminates between steroid receptors. Together these data demonstrate that EDD serves as a coactivator in PR- and VDR-mediated transcription.

2.8 EDD Interacts with CIB, a Protein Potentially Involved in DNA Damage Responses

Further yeast two-hybrid screening was aimed at identifying other proteins involved in the ubiquitinylation or coactivation functions of EDD. When full length EDDM or EDDF5M (aa 889-2799) were used as baits, three clones encoding calcium- and integrin-binding protein/DNA dependent protein kinase interacting protein (CIB/KIP) were isolated: two full-length and another encoding CIB/KIP aa 5-191. CIB is a protein with possible dual roles in the cytoplasm and nucleus (Wu et al, Mutation Research 385, 13-20, 1997; Naik et al, J. Biol. Chem. 272, 4651-4654, 1997; Kauselmann et al, EMBO J 18, 5528-5539, 1999; Shock et al, Biochem. J. 342, 729-735, 1999). The interaction between CIB and full length EDD initially detected in the yeast two-hybrid system (FIG. 10A) was confirmed by pull-down of in vitro translated EDD proteins with GST-CIB (FIG. 10B). Mapping of this interaction using in vitro translated EDD fragments showed that CIB interacts with the carboxy terminal portion of the EDD protein (EDDF3, EDDF3M, FIG. 10C). To obtain evidence for interaction in cells, FLAG-tagged CIB was expressed in HEK 293 cells overexpressing EDD and protein extracts were prepared. EDD protein was detected in FLAG immunoprecipitates from these lysates but not from those of vector transfected cells (FIG. 10D, left panel). GST-CIB fusion protein also interacted with EDD in cell lysates prepared from nuclei of MCF-7 cells expressing endogenous levels of EDD (FIG. 10D, right panel, ‘Control’).

Because we observed EDD in nuclei, a possible nuclear role for CIB was investigated. As CIB was previously found to interact with the DNA damage-sensing enzyme DNA PK (Wu et al, Mutation Research 385, 13-20, 1997), lysates from MCF-7 cells treated with the radiomimetic phleomycin, which induces double strand breaks in DNA, were incubated with GST-CIB fusion protein. Capture of the bound protein revealed significantly less association between EDD and CIB when cells had, been treated with phleomycin, when compared to untreated cells or cells treated with hydroxyurea, Which causes DNA crosslinking (FIG. 10D, right panel). The change in binding was not due to decreases in EDD protein levels which were unchanged (not shown).

These studies show that EDD interacts with a potential ubiquitinylation substrate, CIB, and that this interaction is sensitive to DNA damage. This is the first indication of protein interactions involving eitherEDD or CIB being responsive to DNA damage.

2.9 The Chk2 N-Terminus Interacts with EDD in HeLa Nuclear Extracts

When GSTchk2-N (aa 1-225) (FIG. 11A) was incubated with HeLa nuclear extract, one of the interacting proteins was found by MS sequencing to be EDD (Steve Jackson and Brandi Williams, Wellcome/CRC, Cambridge, UK). We confirmed this by GSTchk2-N pull-downs from MCF-7 cells followed by western blotting to detect EDD protein (FIG. 11B). Immunoprecipitation of chk2 from whole cell extracts prepared from either HEK 293 cells or MCF-7 cells, both of which contain endogenous levels of chk2 and EDD, followed by immunoblotting for EDD confirmed an in vivo association between EDD and chk2 (FIG. 11C).

2.10 EDD Interacts with the FHA Domain of Chk2

When various fragments of ³⁵S-labelled in vitro translated EDD were tested for binding to GSTchk2-N, the carbox-terminal two-thirds of EDD (EDDF5, aa 889-2799) was found to interact. To determine the requirement for the chk2 FHA domain in this interaction, the binding between EDD and GSTchk2-N and two mutants was assessed. The R117A mutant contains a substitution within the FHA domain and involves a residue directly required for phosphothreonine binding. This mutant did not bind EDD (FIG. 12A). The I157T Li-Fraumeni-associated mutant retains the ability to bind phosphothreonine but has been shown to be unable to bind substrates of chk2 such as p53 (Falck et al, Oncogene 20, 5506-5510, 2001) cdc25A (Falck et al, Nature 410, 842-847, 2001), BRCA1 and cdc25C (Li et al, Mol Cell 9, 1045-1054, 2002). Interestingly, the I157T substitution had no effect on binding to EDD (FIG. 12A), suggesting that EDD may instead bind through a phosphothreonine residue. These data were supported by further GSTchk2-N pull-down experiments using MCF-7 nuclear extracts (FIG. 12B).

The requirement of an intact phosphopeptide-binding motif within the FHA domain for EDD binding to the chk2 N-terminal region suggested that the interaction is mediated by a phosphorylated threonine residue within the EDD protein. Although the residue involved has not yet been identified, EDD is phosphorylated in cells as when Flag-tagged EDD was overexpressed in HEK-293 cells in the presence of ³²P-labelled orthophosphate and cell lysates prepared, the resulting Flag immunoprecipitates contained a labelled species corresponding to EDD (FIG. 11C). Further, when HEK-293 or MCF-7 extracts were incubated in the presence of lambda protein phosphatase to remove phosphates, the electrophoretic mobility of EDD was increased, indicating the presence of phosphorylated amino acid residues within the EDD protein.

A phosphopeptide (RWFDT(P)YLIRR) (SEQ ID NO: 46) that binds strongly to the FHA domain was used to further investigate the affinity of the chk2 FHA domain for EDD. Pre-incubation of the GSTchk2-N fusion protein with this peptide at concentrations greater than 50 μM abolished binding between EDD and chk(2-N (FIG. 12D).

2.11 DNA Damage Causes Disruption of the EDD-chk2 Interaction

The role of the EDD-chk2 interaction in the DNA damage response was investigated. MCF-7 cells were treated with the radiomimetic drug phleomycin, which causes double strand breaks in DNA and activation of chk2 via ATM kinase (Matsuoka et al, Science 282, 1893-1897, 1998). A phosphorylation-induced mobility shift of chk2 in response to DNA damage was observed (FIG. 14A). In contrast, no change in either the mobility or levels of EDD protein in MCF-7 nuclear extracts was detectable. However when chk2 was immunoprecipitated from these extracts the amount of EDD associating was considerably reduced following DNA damage and these results were in agreement with GSTchk2-N pull downs from these same lysates (FIG. 14B).

2.12 Depletion of EDD Impairs CHK2 Activation

To examine the role of the EDD-CHK2 interaction in the DNA damage response, cells deficient in EDD were observed for CHK2 activation on DNA damage. RNA interference was used to deplete cells of EDD (as described herein) which were then irradiated and allowed to recover for up to 1.5 h. In cells lacking EDD, DNA damage-induced phosphorylation of CHK2 on T68 was considerably diminished compared to control cells (FIG. 15A). Next, CHK2 kinase activity towards a GST-cdc25C substrate was assayed at various times post-IR in the presence and absence of EDD. These assays showed that activation of CHK2 was delayed in cells lacking EDD and these cells were impaired in their ability to reach maximal CHK2 kinase activity compared to control cells (FIG. 15B).

2.13 EDD and BRCA2 Interactions with chk2 May Have Distinct Roles in the DNA Damage Response

EDD also interacts with BRCA2. EDD was identified along with BRCA2 in a 2 MDa protein complex isolated from nuclear extracts of HeLa cells (R. Shiekhaftar, Wistar Institute, U. Penn, personal communication). We confirmed this association was confirmed by co-immunoprecipitation of EDD and BRCA2 from HEK-293 cell lysates (FIG. 17A). Therefore we were interested to test the possibility of an interaction between chk2 and BRCA2. Indeed when chk2 was immunoprecipitated from MCF-7 nuclear extracts, both EDD and BRCA2 were detected (FIG. 17B). While the interaction between EDD and chk2 was diminished on phleomycin treatment, there was no effect on the amount of BRCA2 associated with chk2. An opposite effect was seen when MCF-7 cells were exposed to the replication block-inducing agent hydroxyurea (HU). Replication block-induced activation of chk2 had no effect on the association with EDD, while the levels of associating BRCA2 were considerably reduced (FIG. 17B). It is possible that these three proteins exist in a common complex. If so, it appears that on DNA damage that results in double strand breaks, EDD tends to dissociate from the complex whereas when the replication block pathway is activated, BRCA2 dissociates. Therefore if EDD and BRCA2 are together in complex with chk2, this complex appears to be differentially affected in response to different types of DNA damage. A complex between BRCA2 and chk2 has not previously been reported and is of great significance in the DNA damage response.

2.14 Mapping the Interaction Between EDD and chk2

The EDD sequence contains a number of threonine residues in a favourable context for FHA binding when phosphorylated (Durocher & Jackson, FEBS Left 513, 58-66, 2002). To pinpoint regions of EDD containing actual binding sites, binding of GSTchk2-N to various ³⁵S-labelled in vitro translated EDD deletion mutants (see FIG. 13A) was examined. In initial experiments, full binding of EDD to chk2 required the carboxyl two-thirds of EDD (EDDF5)—Neither EDDF2 (central third) nor EDDF3 (carboxy third) were sufficient for binding (FIG. 13A).

Removal of either the Hect domain or the RING-like domain from EDDF5 (EDD6 AND EDD7 respectively) did not significantly affect binding (FIG. 13B), suggesting that the region of EDD that binds to the chk2 FHA domain lies between amino acids 1406 and 2526.

2.15 Mapping the Interaction Between EDD and BRCA2

To pinpoint regions of EDD containing actual binding sites for BRCA2, binding of in vitro translated BRCA2 to various ³⁵S-labelled in vitro translated EDD deletion mutants (see FIG. 13A) is examined.

Additionally deletion mutants of BRCA2 are generated and labelled with 35S. The ability of the full length EDD protein is tested to determine the region of BRCA2 to which EDD binds. Such information facilitates the analysis of the BRCA2-EDD complex and the Chk2-EDD complexes in order to determine whether these interactions form a single complex (ie BRCA2-EDD-Chk2), or instead whether these interactions form distinct complexes.

In order to further characterise the interactions between EDD and BRCA2, these proteins are both expressed in HCT-15 cells, that lack Chk2 expression. Following expression of both proteins in HCT-15 cells, antibodies to EDD are used in immunoprecipitation experiments. The captured protein complexes are separated using SDS-PAGE and BRCA2 levels determined using an anti-BRCA-2 antibody.

HCT-15 cells expressing EDD and BRCA2 are then transfected with an expression vector that expresses Chk2 and the coimmunoprecitptation experiments repeated, in order to determine the effect of Chk2 on the association of EDD and BRCA2.

2.16 Functional Consequences of EDD-BRCA2-CHK2 Interaction

Ubiquitinylation assays are be used to investigate whether EDD is activated in response to DNA damage and whether EDD targets BRCA2 for ubiquitinylation and subsequent degradation. The assay utilizes in vitro translated or immunoprecipitated EDD with reactions performed in the presence of GST- or His-tagged ubiquitin, E1 and the F2 UbcH5b with TopBP1 as positive control substrate. EDD activity is determined in the presence and absence of ionizing radiation. This assay is also used to test other candidate substrates for ubiquitinylation by EDD such as CIB, or the Gli1, −2 and −3 proteins.

Depletion of EDD from MCF-7 breast cancer cells using established RNAi protocols (described herein) is used to determine the degree to which BRCA2 is targeted by EDD for ubiquitn-mediated degradation. The effects of DNA damage and proteasome inhibitors on BRCA2 protein levels are monitored using western blotting.

In order to determine the role of EDD in BRCA2-mediated repair of double strand DNA is lesions the frequency of radiation-induced BRCA2-RAD51 nuclear foci formation in MCF-7 or HEK-293 cells with either normal EDD levels or those transfected with EDD short interfering RNAs (siRNAs) is determined. Complex formation between BRCA2 and RAD51 is also monitored by co-immunoprecipitation experiments.

2.17 The EDD-BRCA2-CHK2 Interaction in the Context of DNA Damage.

To examine the interaction in the context of DNA damage signaling via the ATM-mediated pathway, cells are treated with ionising radiation at doses of between 4 and 12 Gy, and the cells allowed to recover for periods of 0-6 h. Activation of CHK2 and other downstream responses to DNA damage are monitored by immunoblotting to detect the levels of CHK2 phosphorylated at T68 and also for p53 accumulation where appropriate.

Furthermore, hydroxyurea is used to study the ATR-mediated response pathway of CHK2 stimulabon and its effects on the EDD-BRCA2-CHK2 interactions.

2.18 Significance of EDD-BRCA2-chk2 Interaction—a Working Model

EDD dissociates from chk2 on DNA damage. Therefore EDD may normally regulate chk2 and needs to be removed for chk2 activation. Overexpression of EDD, as observed in breast cancers for example, might then inhibit the activation of chk2 in response to DNA damage, thus leading to either replication of defective DNA or continued proliferation of cells with an abnormal chromosomal complement. These conditions could foster further mutation and lead to cancer or tumour progression. Conversely, in EDD knockout mouse embryos or cells subjected to EDD siRNAs, we propose that chk2, in the absence of the inhibitory effects of EDD, might be dysregulated and thus cause prolonged inappropriate cell cycle arrest.

It is therefore important to understand the precise pathways impinging upon the chk2-EDD-BRCA2 interactions. In any case, the interaction of EDD with these two tumour suppressor proteins underscores a potential role for EDD in cancer development and progression. Specifically, the well established role of CHK2 and BRCA2 in DNA damage repair and cell cycle arrest strongly suggest that EDD may play a role in such pathways.

2.19 Discussion

This study demonstrates a new functional role for the nuclear protein EDD. A progestin regulated gene, EDD itself has the ability to potentiate PR transcriptional activity. In addition, EDD may play a role in DNA damage signalling as suggested by complex formation with CIB, a DNA PK-binding protein, an interaction that is sensitive to DNA damage.

The present study showed that EDD is a nuclear protein, most likely arising from a direct interaction with importin α via two NLSs within the N-terminus of EDD. The HECT domain, which has reversible ubiquitin binding activity in EDD and other E3 ligases (Callaghan et al, Oncogene 17, 3479-3491, 1998), is also associated with a separate role in transcriptional coactivation in related proteins: Rsp5/hRPF1 and E6-AP coactivate ligand-dependent nuclear receptor activity (Imhof et al, Mol. Cell Biol. 16, 2594-2605, 1996; Nawaz et al, Mol. Cell Biol 19, 1182-1189, 1999), while Tom1p is required for transcriptional regulation of certain yeast genes (Saleh et al, J. Mol. Biol. 282, 933-946, 1998) and UREB1 enhances transcription from the rat preprodynorphin gene (Gu et al, Mol. Brain Res. 24, 77-88, 1994) but suppresses p53 transactivation of target genes (Gu et al, Oncogene 11, 2175-2178, 1995).

EDD potentiates PR transactivation to a level comparable to that seen for the p160 coactivator SRC-1. EDD has a distinct selectivity profile, being able to coactivate PR and VDR but not ER, in a ligand-dependent manner. This is in contrast to the HECT ligase E6-AP which coactivates a range of hormone receptors including ER, PR, AR and GR (Nawaz et al, Mol. Cell Biol 19, 1182-1189, 1999). Rsp5 also shows some selectivity, coactivating transcription by PR and GR but not ER (Imhof et al, Mol. Cell Biol. 16, 2594-2605, 1996). EDD is unique among HECT ligases however in that enhancement of PR transactivation by EDD raises the intriguing possibility of a positive feedback loop, as EDD itself is a progesterone-regulated gene (Callaghan et al, Oncogene 17, 3479-3491, 1998). Thus, overexpression of EDD seen in some breast cancers could increase the sensitivity of PR-positive tumours to lower levels of progestins.

Coactivation by EDD, like E6-AP, Rsp5/hRPF1 and Tom1p, is independent of ubiquitin binding ability of the HECT domain (Saleh et al, J. Mol. Biol. 282, 933-946, 1998; Imhof et al, Mol. Cell Biol. 16, 2594-2605, 1996; Nawaz et al, Mol. Cell Biol 19, 1182-1189, 1999). These findings are somewhat surprising in the light of evidence that ubiquitinylation is intimately involved in the process of transcriptional activation. Like many other transcription factors, several nuclear receptors including ER, PR and VDR are down-regulated by the 26S proteasome (Nawaz et al, Proc. Natl. Acad. Sci. USA 96, 1858-1862, 1999; Alarid et al, Mol. Endocrinol 13, 1522-1534, 1999; Lange et al, Proc. Natl. Acad. Sci. USA 97, 1032-1037, 2000; Lonard et al, Mol. Cell 5, 939-948, 2000; Masuyama et al, Biochem 71, 429-440, 1998) and coactivator binding appears necessary for this degradation. Inhibition of the proteasome diminishes transcriptional activity by steroid receptors ER and PR (Dennis et al, Front Biosci. 6, 954-959, 2001) and more general implications come from studies showing that the 19S proteasome subunit is required for transcription elongation (Ferdous et al, Mol. Cell 7, 981-991, 2001). Furthermore, the carboxy terminal tail of RNA polymerase II itself is a target of ubiquitin-mediated proteolysis (Zhu et al, Nature 400, 687-693, 1999; Huibregtse et al, Proc. Natl. Acad. Sci. USA 94, 3656-3661, 1997). It may be that EDD and these other HECT ubiquitin ligases can still perform some function in the ubiquitinylation cascade without themselves having a catalytically active HECT domain. EDD appears to be the only E3 ligase to possess both a RING-like zinc finger domain and a HECT domain and we cannot rule out the possibility that coactivation by EDD is mediated through the RING-like or UBA domains.

The mechanism of coactivation by E6-AP, as for the p160 family, has been attributed to direct coactivator-receptor interaction, providing either bridging or enzymatic activities to the transcriptional complex. Many steroid receptor coactivators possess histone acetyl transferase (HAT) activity, but when compared to p300 little or no HAT activity was associated with EDD (our unpublished data). Two regions of E6-AP contain LXXLL receptor-binding motifs and both of these regions interact with PR (Nawaz et al, Mol. Cell Biol 19, 1182-1189, 1999). A search of the EDD sequence revealed one N-terminal, one C-terminal and three centrally located LXXLL domains (FIG. 5A). Furthermore, the N-terminal and centrally located motifs lie in regions of high homology to HYD. However, the N-terminal motif, in the region with the strongest binding to PR(CDE), was not required for the interaction. Nevertheless, direct interaction between other N-terminal sequences of EDD and PR may partially explain the observed effects of EDD on PR transactivation. In their studies on the role of ubiquitinylation in transcriptional enhancement, Salghetti et al found that mono-ubiquitinylation of the VP16 transcriptional activation domain was sufficient for transcriptional activity (Salghetti et al, Science 293, 1651-1653, 2001). Interestingly, another study found that the UBA domain might bind such mono-ubiquitinylated proteins and thus prevent the formation of multi-ubiquitin chains (Bertolaet et al, Nat. Struct. Biol. 8, 417-422, 2001), raising a possible mechanism for coactivation via PR stabilisation by EDD. However, we found that the UBA domain was not required for interaction between EDD and PR in vitro, although we cannot rule out a separate role for the UBA domain in PR coactivation by EDD.

In addition to the UBA domain, the zf-UBR1 domain of EDD is also likely to be involved in protein-protein interactions. The zf-UBR1 domain coincides with the type 1 site in UBR1 proteins, a binding site specific for N-end rule substrates with basic N-terminal residues (Kwon et al, Proc. Natl. Acad. Sci. USA 95, 7898-7903, 1998). This zf-UBR1 domain is critical for function of the calossin-like RING-H2 finger protein, BIG and it therefore may also have a role in substrate recognition and binding in EDD family members. Other HECTs have substrate interaction domains distinct from the HECT domain (eg. the WW domain (Huibregtse et al, Proc. Natl. Acad. Sci. USA 94, 3656-3661, 1997)). Unfortunately attempts to use the UBA and zf-UBR1 as baits for yeast two-hybrid analysis were unsuccessful due to autoactivation and indiscriminate binding respectively, so the precise functions of these regions in the EDD protein and their role, if any, in coactivation remain elusive.

Using cell lysates EDD and CIB were shown to interact in MCF-7 and HEK 293 cells. CIB is 58% and 56% homologous with other EF-hand proteins calmodulin and calcineurin B, respectively and may function as a calcium-dependent regulatory subunit of a kinase or phosphatase (Naik et al, J. Biol. Chem 272, 4651-4654, 1997). Interactions of CIB with five other proteins have been described: DNA PK (Wu et al, Mutation Research 385, 13-20, 1997), integrin allb (Naik et al, J. Biol. Chem 272, 4651-4654, 1997), the cell cycle regulatory polo-like kinases, Snk and Fnk (Kauselmann et al, EMBO J. 18, 5528-5539, 1999; Tsuboi, J. Biol. Chem. 277, 1919-1923, 2002) and presenilin 2 (Stabler et al, J. Cell Biol. 145, 1277-1292, 1999). Snk and Fnk are activated by progesterone in maturing frog oocytes (Duncan et al, Exp. Cell Res. 270, 78-87, 2001) and have roles in both G1 and mitotic phases of the cell cycle and CIB could affect the activity of these kinases. CIB is found in both the nucleus and cytoplasm and its subcellular localisation can be influenced by association with its interacting partners and by calcium levels (Kauselmann et al, EMBO J. 18, 5528-5539, 1999; Stabler et al, , J. Cell Biol. 145, 1277-1292, 1999) but its role in the nucleus is unexplored. Interaction with DNA PK would implicate CIB in the response to DNA double strand break sensing and repair. We found that GST-CIB pull-down of EDD after treatment with the radiomimetic phleomycin caused a decrease in the amount of associating EDD while the levels of EDD remained unchanged. Interestingly a recent report links EDD and ubiquitinylation of another protein involved in DNA repair, the topoisomerase II associated protein, TopBP1 (Honda et al, J. Biol. Chem 277, 3599-3605, 2002). CIB interacts with PLK in the activation of CHK2. In the light of these data and our findings that CIB is a potential target of the proteasome, experiments are currently under way to determine the possible involvement of EDD and CIB in the cellular response to DNA damage.

These data, identifying a role for EDD in transcriptional control and DNA damage (Honda et al, J. Biol. Chem 277, 3599-3605, 2002), together with our other data demonstrating embryonic lethality in EDD^{-/-} mice and frequent allelic imbalance at the EDD locus in diverse human cancers, provide strong evidence that EDD plays a pivotal role in normal cellular physiology and when disregulated has important consequences for development and potentially tumourigenesis.

### EXAMPLE 3

### Targeted Disruption Of Edd In Mice Causes Embryonic Lethality Due To A Generalised Failure Of Cell Proliferation, Generalised Apoptosis and Defective Vascularization of the Yolk Sac

3.1 Targeted Disruption of the Mouse Edd Gene

Edd-deficient (Edd^{-/-}) mice were generated by homologous recombination in embryonic stem (ES) cells. The Edd targeting construct was designed to delete 3.4 kb of Edd genomic DNA containing 61 bp of exon 1 (immediately downstream of the ATG translation start site) and 3.3 kb from the following intron, and replace it with a 6.5 kb βGal-GFP-Neo^{r} expression cassette (FIG. 18A). This replacement was designed to produce an effectively null Edd allele and express a βGal-GFP fusion protein under control of the normal Edd gene upstream regulatory elements. The targeting construct was electroporated into 129/SvJ ES cells and several neomycin-resistant clones were isolated and screened for disruption of the Edd gene by Southern blot analysis (FIG. 18B). Restriction of wild-type genomic DNA with BamHl produced a 6 kb fragment. Following homologous recombination with the targeting vector, and replacement of the 5′ BamHl site, a smaller 4.2 kb hybridising fragment was generated following restriction. Therefore, a correctly targeted ES cell clone produced 6 kb and 4.2 kb fragments following digestion with BamH1, representing the wild type and mutated alleles respectively (FIG. 18B). Two independently targeted ES cell clones (1 B2 and 5C6) were used to generate chimeric mice by injection into blastocyst stage C57BL/6 embryos. Following backcrossing with C57BL/6 mice, F1 animals were again analysed by Southern blotting to confirm germline transmission of the mutated Edd allele. Routine genotyping of adult tail and embryo yolk sac DNA by PCR generated an amplicon of 600 bp from wild-type DNA (using primers 1 (SEQ ID NO: 50) and 2 (SEQ ID NO: 51)). However, the primer 1 recognition sequence is deleted following insertion of the targeting vector and hence the mutated allele is detected as a 440 bp amplicon (using primers 2 (SEQ ID NO: 51) and 3 (SEQ ID NO: 52)) (FIG. 18B).

Northern blot analysis showed expression of Edd mRNA in a wide range of wild-type (WT) adult mouse tissues, similar to the expression pattern of human EDD. Reduced Edd mRNA expression was observed in most tissues analysed from heterozygous (Edd^{±}) animals (FIG. 18D). However, western blot analysis showed only slightly decreased Edd protein expression in Edd^{±} adult testis and E10.5 embryonic tissue (FIG. 15D). Edd expression was not detected in tissue from Eddy^{-/-} E10.5 embryos by either western blotting or IHC (FIGS. 18D and 18E), confirming the efficacy of the targeting strategy. Edd was detectable by IHC in most cells of developing WT embryos (FIG. 18E), with the exception of hematopoietic cells. Neither GFP or LacZ expression was detectable in Edd^{±} or Edd^{-/-} embryos, most likely due to either very low βGal/GFP expression, or production of a non-functional fusion protein.

3.2 Phenotype of Heterozygous (Edd^{±}) Mice

Both male and female Edd^{±} mice on a mixed 129 SV/J×C57BL/6 background were aged up to 80 weeks and showed no apparent tumorigenic phenotype, with comparable fertility and growth rates to wild-type controls.

3.3 Homozvgous Null Edd Mutation (Edd^{-/-}) Results in Embrvonic Lethality

Edd^{±} mice were interbred, and genotypes of offspring determined by PCR analysis of ear, tail or embryo yolk sac (FIG. 18B). No homozygous mutant (Edd^{-/-}) animals were detected from 274 offspring analysed, indicating that complete Edd deficiency (homozygosity for the Edd null mutation) causes embryonic lethality (Table 6). To characterize the developmental stage at which Edd deficiency caused embryonic lethality we analysed embryos from heterozygous intercrosses at various stages of gestation (Table 6). Mendelian ratios of wild-type, Edd^{±} and Edd^{-/-} embryos were observed up to E10.5. No viable Edd^{-/-} embryos were observed beyond E10.5. Some Edd^{-/-} embryos were present in litters at E11.5 but these were partially resorbed and scored as non-viable. A number of empty decidua were observed at E11.5-12.5 and these may have arisen from resorption of Edd^{-/-} embryos at earlier stages of gestation. These data demonstrate that homozygosity for the Edd null mutation results in embryonic lethality before E11.5 and indicate that Edd is essential for post-implantation embryonic development.

Heterozygous mice and Edd^{-/-} embryos generated from the two independently targeted ES cell lines showed identical phenotypes (data not shown). Furthermore, no difference was observed in Edd^{-/-} embryos following excision of the neomycin resistance (Neo^{r}) gene from the targeted allele by crossing Edd^{±} mice with mice transgenic for Cre recombinase.

3.3 Morphology of Edd^{-/-} Embryos

Edd^{-/-} embryos are slightly growth retarded as early as E7.5 when compared to wild-type and Edd^{+/-} littermates (FIG. 19). By E8.5 Edd^{-/-} embryos are clearly developmentally retarded, lagging at least 0.5 days behind the development of WT and Edd^{±} littermates. By E9.5 and E10.5, Edd^{-/-} embryos display severe growth defects, the most apparent being the absence of turning which occurs in WT embryos around E9 (FIG. 19). In addition, the head is small and embryonic structures forming the jaw region (branchial arch) are significantly under-developed. Edd^{-/-} embryos are frequently observed with a swollen pericardium, indicating osmotic imbalance within the embryo. Many Edd^{-/-} embryos also display a bulbous allantois indicating failure of chorioallantoic fusion and placentation (FIG. 19). Upon histological examination, blood cell pools can be seen in several regions of Edd^{-/-} embryos. Specifically, a pool of blood cells can be seen within the pericardial cavity, indicating pericardial effusion (data not shown). Edd^{-/-} embryos also appear to contain far less neural epithelium than WT and the epithelial structure is disorganised. In short, defects in Edd^{-/-} embryonic tissue are widespread and do not appear to be restricted to a specific organ system or cell type.

3.4 Failure of Proliferation in Edd^{-/-} Embryos

To assess the cause of growth retardation of Edd^{-/-} embryos, cellular proliferation in developing embryos was examined by measuring BrdU incorporation into DNA during S-phase. Pregnant females were injected with 10 μg/g BrdU 1 hr before sacrifice, embryos collected and fixed in 4% paraformaldehyde, and IHC using anti-BrdU (Dako) antibodies was then performed on paraffin sections. Positive staining indicates incorporation of BrdU into newly synthesised DNA in proliferating cells. At E8.5, the earliest stage at which a clear defect is observed in Edd^{-/-} embryos, there was no significant difference in mitotic index between WT (76%±2.9) and Edd^{-/-} embryos (73%±3.8) (FIG. 21). By E9.5 a marked decrease in proliferation was observed in Edd^{-/-} embryos. Some proliferating cells were detected (9%±2.8) but this number was dramatically lower than levels observed in WT littermates (73%±3.1) (FIG. 21). Interestingly, BrdU incorporation in Edd^{-/-} embryos after E9.5 was restricted almost exclusively to hematopoletic cells. Significantly, these were the only cells found not to express EDD in wild-type embryos (FIG. 20). Proliferation remained high in WT embryos at E10.5 (67%±2.9) while almost no proliferation was detected in Edd^{-/-} embryos (3%±1.8). Hence, complete Edd deficiency results in a widespread proliferative defect in most cells of the developing mouse embryo after E8.5 (FIG. 21), reflecting the widespread expression of Edd in WT embryos.

3.5 Increased Apoptosis in Edd^{-/-} embryos

The observation of numerous condensed nuclei in histological sections from E10.5 Edd^{-/-} embryos, suggested that along with a proliferative block, apoptosis was also contributing to the embryonic lethality observed in Edd-deficient embryos. TUNEL staining was used to examine apoptosis in histological sections from embryos between E8.5-10.5 and no significant difference was observed in numbers of TUNEL-positive nuclei at E8.5 (FIG. 22A). However, numerous condensed nuclei were observed in Edd^{-/-} tissue at E9.5 and E10.5 (data not shown) and significantly higher levels of TUNEL-positive cells were visible in Edd^{-/-} embryos compared to WT littermates at E9.5 and E10.5 (FIG. 22A).

To further characterise the apparent activation of apoptosis in Edd^{-/-} embryos, IHC staining using an antibody that detected the cleaved (i.e. active) form of caspase-3 (R&D Systems, Minneapolis, USA) was also performed. As with BrdU and TUNEL staining, no significant difference in staining for active caspase-3 was observed between WT and Edd^{-/-} embryos at E8.5 (13% and 16% respectively). However, significantly higher staining for active caspase-3 was observed in Edd^{-/-} embryos compared to WT littermates at both E9.5 (44% vs 6%) and E10.5 (46% vs 6%) (FIG. 22B), coinciding with the increased presence of both condensed nuclei and TUNEL staining in these embryos. Hence, activation of caspase-3 mediated apoptosis coincides with the block in proliferation observed in Edd^{-/-} embryos, indicating that the retarded development observed at E9.5-E10.5 results from a combination of decreased cell proliferation and increased cell death.

3.6 Defective Yolk Sac Vascularisation in Edd^{-/-} Embryos

The widespread defects observed in Edd^{-/-} embryos indicate a potential failure of yolk sac vasculogenesis or placentation, leading to disrupted nutrient exchange. We therefore examined the morphology of Edd^{-/-} and WT yolk sacs. From E9.5 onwards, Edd^{-/-} yolk sacs are clearly less well vascularised than their Edd^{+/-} or WT littermates, suggesting a defect in yolk sac vasculogenesis. Numerous large vessels are visible in WT and heterozygous yolk sacs at E10.5, whereas very few, smaller vessels are visible in Edd^{-/-} yolk sacs at this stage (FIG. 23).

High magnification of histological sections from WT and Edd^{-/-} yolk sacs at E9.5 (FIG. 24) shows distinct vascular channels containing blood cells (b) are visible in WT yolk sac, whereas EDD-null yolk sacs display enlarged channels with unusual separation of mesoderm (m) and endoderm (e) and few blood cells. These data indicate that the differentiation of visceral mesoderm to vascular endothelium may be disrupted by EDD deficiency.

3.7 The Edd^{-/-} Phenotype is p53 Independent

Given the potential role of Edd in DNA damage response, decreased cell proliferation and increased apoptosis in Edd^{-/-} embryos, we attempted to ascertain whether a genetic relationship exists between Edd and p53. Edd^{±} mice were crossed with mice heterozygous for a truncating mutation in p53 (as described by Jacks et al, Curr Biol 4:1-7, 1994) and Edd/p53 double heterozygous animals were subsequently produced. Embryos from intercrosses of these double heterozygotes were examined and no difference was observed in the survival of Edd^{-/-} embryos on a p53^{-/-} background (Table 7). Edd^{-/-/p}53^{-/-} embryos at E10.5 showed similar morphology to Edd^{-/-}/p53^{+/+} embryos and all Edd^{-/-}/p53^{-/-} embryos observed at E11.5 were partially resorbed, demonstrating that decreased cell proliferation and activation of apoptosis in Edd^{-/-} embryos are p53-independent.

### EXAMPLE 4

### The Role of EDD in Mammary Gland Development and Tumorigenesis

4.1 Production of a Conditional Knock-Out of the Mouse EDD Gene

Conditional Edd-deficient (Edd^{-/-}) mice are generated by homologous recombination in embryonic stem (ES) cells. The Edd targeting construct is designed to flank exon 1 (which contains the ATG translation start site for EDD) of the EDD gene with Cre recombinase recognition sites (loxP). Expression of Cre recombinase in the tissue/s of interest causes excision of exon 1, thereby silencing EDD in those tissue/s.

The targeting construct is electroporated into 129/SvJ ES cells. A portion of these cells are then electroporated with an expression vector that expresses Cre recombinase and clones that show disruption of the Edd gene by Southern blot analysis determined. A portion of the selected cells, that did not express Cre recombinase (ie those cell with an intact EDD gene) are used to generate chimeric mice by injection into blastocyst stage C57BL/6 embryos. Following backcrossing with C57BL/6 mice, F1 animals are analysed by Southern blotting and/or PCR (to detect the presence of the loxP sites) to confirm germline transmission of the mutated Edd allele.

Mice expressing Cre recombinase only in mammary tissue are then generated. An expression construct with the nucleic acid encoding Cre recombinase (SEQ ID NO: 49) under control of the MMTV LTR (SEQ ID NO: 48), which drives expression only in mammary tissue (Wagner et al, Nucl. Acid Res. 25(21): 4323-4330, 1997 and Ahmed et al, Cancer Res., 62(24): 7166-7169, 2002). This gene construct is then microinjected into the pronucleus of a fertilised oocyte and the oocyte introduced into the uterus of a pseudopregnant female C57BL/6 mouse. All mice born are screened for presence of the transgene by Southern hybridisation and/or PCR to detect the nucleic acid that encodes Cre. Those mice that are found to carry the transgene are bred with WTr C57BL/6 mice, and subsequent generations are screened for expression of Cre recombinase in mammary tissue using an anti-Cre antibody (Novagen, Madison, Wis., USA). Multiple tissues of positive mice are subsequently screened to ensure that the expression of Cre is limited to the mammary tissue.

Transgenic mice expressing Cre and mice carrying f/oxed EDD are then crossed to generate mice that have had exon 1 of EDD excised in mammary tissue. Expression of EDD in several tissues (including mammary tissue) is determined by both Western blotting and immunohistochemistry as described in Example 3. Those mice that do not express EDD in mammary tissue, but maintain expression of EDD in all other tissues in which EDD is known to be expressed. These mice are then analysed to establish the role of EDD in mammary cell development and tumorigenesis.

4.2 Role of EDD in Mammary Gland Development

Female mice deficient for EDD in mammary tissue are analysed to determine mammary gland architecture and function. Pregnant female mice (wt and EDD^{-/-}) are sacrificed every 5 days during pregnancy (where day 0 is considered the date on which a post-coital plug is first detected). Mammary tissue is then dissected from these animals and analysed using whole-mount microscopy to determine terminal end bud density, intemodal duct length, branching pattern and alveolar bud formation, and to determine the effect of EDD on these parameters (essentially as described in Hovey et al, Mol. Endocrinol. 17(3): 460-473, 2003 and references therein). Additionally, the effect of EDD are analysed at weekly intervals for three weeks during lactation and at 5 day intervals for 6 weeks following weaning.

At all time points mammary glands are also sectioned and stained with haemotoxylin and eosin to facilitate examination of the cellular structure of the glands. Additionally, glands are analysed for expression of EDD and EDD interacting proteins, such as, for example, BRCA2, CHK2, TopB1 and CIB.

As recent studies have suggested that the drosophila homolog of EDD (hyd) is capable of regulating the expression of hedgehog and decapentaplegic, mammary tissue isolated from EDD^{-/-} mice are analyzed for hedgehog signaling pathway components. eg ihh, shh, dhh, Gli1, Gli2, Gli3, BMP-2, BMP-4 and patched proteins.

In order to determine the effect of EDD in the stromal and epithelial compartments of mammary tissue, epithelial tissue from an EDD−/− mice are transplanted into a fat pads of a WT mouse, and epithelial tissue from a wt mouse is transplanted into a fatpad of a EDD^{-/-} mouse (essentially as described in Naylor and Ormandy. Dev. Dyn, 225(1): 100-105, 2002 and references cited therein).

4.3 Role of EDD in Mammary Tumorigenesis

In order to assess carcinogenesis in mice carrying a targeted disruption of EDD in the mammary gland, mice are aged for up to a period of 2 years. At various stages mice are sacrificed and analyzed for the development of spontaneous mammary tumors. The rate of tumor development in EDD^{-/-} mice and wt mice at these stages are then compared to determine the effect of EDD on the development of mammary tumors in ageing mice.

Additionally, EDD^{-/-} mice are crossed with the MMTV/c-myc transgenic mice (Romieu-Mourez et al, Mol. Cell Biol, 23(16): 5738-5754, 2003). or the MMTV/wnt-1 transgenic mice (Bocchinfuso et al, Cancer Res 59(8): 1869-1876, 1999) (both of which show a high incidence of mammary tumor formation). WT, transgenic and transgenic/knockout mice are then analyzed to determine the effect of EDD on latency and frequency of tumor formation. Mammary glands are isolated from the mice at various stages of pregnancy and lactation and various ages and analyzed using whole mount analysis to determine the number of hyperplastic alveolar nodules, hyperplasias and tumors.

4.4 Overexpression of EDD in Mammary Tissue

In order to determine the effect of EDD on the development of mammary tissue and mammary tumorigenesis mice overexpressing EDD in this tissue are generated. An expression construct with the nucleic acid encoding EDD (SEQ ID NO: 1) under control of the MMTV LTR (SEQ ID NO: 48), which drives expression only in mammary tissue is produced. This gene construct is then microinjected into the pronucleus of a fertilised oocyte and the oocyte introduced into the uterus of a pseudopregnant female C57BL/6 mouse. All mice born are screened for presence of the transgene by Southern hybridisation and/or PCR to detect the transgenic construct encodes mammary tissue specific EDD. Those mice that are found to carry the transgene are bred with WT C57BL/6 mice, and subsequent generations are screened for overexpression of EDD in mammary tissue using Western Blotting as described in Example 3. Multiple tissues of positive mice are subsequently screened to ensure that the overexpression of EDD is limited to the mammary tissue.

4.5 Role of EDD in Mammary Development

Female mice overexpressing EDD in mammary tissue are analysed to determine mammary gland architecture and function. Mammary tissue is isolated from pregnant female mice (wt and tgEDD^{±}) every 5 days during pregnancy, in addition to weekly intervals for three weeks during lactation and at 5 day intervals for 6 weeks following weaning Dissected mammary tissue is then analysed using whole-mount microscopy to determine terminal end bud density, intemodal duct length, branching pattern and alveolar bud formation, and to determine the effect of EDD on these parameters (essentially as described in Example 4.2).

At all time points mammary glands are also sectioned and stained with aemotoxylin and eosin to facilitate examination of the cellular structure of the lands. Additionally, glands are analysed for expression of EDD and EDD interacting proteins, such as, for example, BRCA2, CHK2, TopB1 and CIB, in addition to hedgehog signaling pathway components. eq ihh, shh, dhh, Gli1, Gli2, Gli3, BMP-2, BMP-4 and patched proteins.

4.3 Role of EDD in Mammary Tumorigenesis

In order to assess carcinogenesis in mice overexpressing EDD in the mammary gland, mice are aged for up to a period of 2 years. At various stages mice are sacrificed and analyzed for the development of spontaneous mammary tumors. The rate of tumor development in tgEDD^{±} mice and wt mice at these stages are then compared to determine the effect of EDD on the development of mammary tumors in ageing mice.

### EXAMPLE 6

### Targeted Downregulation of EDD Expression Using siRNA Inhibits Cell Proliferation and Disrupts Cell-Cell Contacts

5.1 Method

An siRNA was designed to inhibit the expression of EDD (sequence: sense 5′-GCAGUGUUCCUGCCUUCUUdTdT-3′ (SEQ ID NO: 47), anti-sense 5′-dTdTCGUCACMGGACGGMGM-3′ (SEQ ID NO:48)), siRNA was synthesised, annealed and HPLC purified by Xeragon (Zurich, Switzerland). 2.2×10⁶ MCF-7 cells or 2.6×10⁸ HEK-293 cells were plated in a 15 cm² dish and grown overnight. The normal breast epithelial cell lines HMEC 184 and MCF-10-A were grown in MCDB 170 media (Gibco) with 1% pituitary extract as an additive.

For transfection of MCF-7 or HEK-293 cells were washed with serum-free medium and 15 ml of serumfree medium was placed on the cells. 29 μ1 of Oligofectamine (Life Sciences) was diluted in 220 μl Optimem (Life Sciences) (incubated for 5 min) and 20.5 μl siRNA (20 μM) was diluted in 2.5 ml Optimem. The siRNA solution and Oligofectamine solutions were gently mixed and incubated for 24 min. The combined solution was added to the 15 cm² plate of cells in serum-free medium. After 4 hrs 15 ml of medium containing 30% fetal calf serum was added to the cells. After another 24 hrs cells were split into 10 cm² plates for each time point treatment.

For transfection, HMEC 184 and MCF10-A cells were plated at 1.2×10⁶ cells/15 cm plate. Transfection conditions were the same as above except HMEC 184 and MCF10-A cells were transfected in pituitary extract-free medium and after 4 h 2% pituitary extract-containing medium is added. 8 hrs after transfection this medium was replaced with 1% pituitary extract-containing medium

A siRNA targeted against green fluorescence protein (GFP) was used as a negative control.

5.2 Results

Transfection of EDD RNAi in all cell lines resulted in substantial loss of EDD protein as assessed by Western blotting with an anti-EDD antibody (FIG. 25). After transfection with EDD siRNA, changes in cell morphology were seen in HMEC 184 cells and MCF-10A cells. This change was first observed two days after RNA interference was performed. Cells transfected with EDD siRNA showed reduced and altered cell-cell contacts, cell shape was altered and cells were disorganised, compared to control cells (FIG. 26). Control cells tended to be elongated, making cell-cell contacts with neighbouring cells along the length of the cells, forming a sheet. Cells transfected with EDD siRNA made fewer connections with neighbouring cells, while their more rounded shape prevents them from making connections along the length of the cells.

Cell morphology was further analysed using immunofluorescent antibodies to detect proteins involved in cell-cell contacts (β-catenin) and organisation of the cytoskeleton (actin). -catenin staining in HMEC 184 cells showed differences at cell-cell contacts after depletion of EDD. Staining in control cells was even along the cell-cell contact (FIG. 27), whereas this region in cells transfected with EDD siRNA showed reduced expression of β-catenin and a patchier staining (FIG. 27). 67% of cell-cell contacts between 184 cells depleted of EDD had patchy expression (compared with 36% in control). Further examination showed that EDD RNAi causes a relocalization of β-catenin from the cell periphery to the cell nucleus (FIG. 28).

Actin filaments in HMEC 184 control cells were coordinated with actin in adjacent cells (FIG. 29), whereas this organisation between neighbouring cells was not seen in cells transfected with EDD siRNA (FIG. 29).

Similar alterations of β-catenin and actin were seen in MCF-10A cells depleted of EDD.

### EXAMPLE 6

### Identification of Downstream Effects of EDD Silencing

To ascertain biochemical pathways that are affected by EDD activity in cells, transcript profiling experiments were performed using Affymetrix DNA microarrays. HMEC 184 and MCF-7 cells were transfected with siRNA (EDD or GFP) as described in Example 5.1. Loss of EDD was confirmed by Western or Northern blot.

Cells were harvested after 24 hours and 48 hours and RNA purified. RNA was then reverse transcribed using an oligo(dT) anchored oligonucleotide that additionally comprised a T7 promoter sequence. Isolated cDNA was then transcribed in vitro using the T7 MEGAscript kit (Ambion, Austin, Tex., USA) according to manufacturer's instructions. Transcription was performed with biotinylated nucleotides (Bio-11-CTP and Bio-16-UTP) to facilitate detection of the transcribed nucleic acid.

Experiments were performed in triplicate and pooled for the Affymetrix probe. Affymetrix U133A chips were used in all experiments.

Data analysis identified genes which had decreased or increased mRNA expression in response to EDD depletion by RNAi.

As shown in Table 8 the expression of a variety of genes that are associated with cell cycle control (eg. Cyclin D3), oncogenes (eg R-ras) and genes associated with cell-cell contact and cell morphology (eg Collagen IVα1) is modulated by the silencing of EDD. These changes in gene expression potentially explain the morphological changes observed in HMEC 184 and MCF-7 cells as a result of EDD silencing (ie. altered cell proliferation, altered cell-cell contact, and altered β-catenin and actin localization).

TABLE 1
Allelic status of polymorphic microsatellites at 8q22.3-24.1 in five tumor types.¹

| | D8S326 | CEDD | D8S257 | D8S300 | D8S545 | D8S85 | MYC.PCR3 | D8S198 |
|---|---|---|---|---|---|---|---|---|
| Ovarian Malignant | 22/55 (40) | 22/46 (48) | 17/41 (41) | 12/31 (37) | 13/45 (29) | 13/39 (33) | | |
| Serous | 12/25 (48) | 16/22 (73) | 11/19 (58) | 7/14 (50) | 6/18 (33) | 5/14 (36) | | |
| Endometrioid | 7/17 (41) | 4/11 (40) | 4/11 (40) | 2/9 (22) | 5/16 (31) | 5/14 (36) | | |
| Mucinous | 1/7 (14) | 1/8 (13) | 0/5 (0) | 1/3 (33) | 1/6 (16) | 1/5 (20) | | |
| Other² | 2/6 (33) | 1/5 (33) | 2/6 (33) | 2/5 (40) | 1/5 (16) | 2/6 (33) | | |
| Ovarian Benign | 0/17 (0) | 0/10 (0) | 0/12 (0) | 0/9 (0) | 1/16 (6) | 1/9 (11) | | |
| Ovarian Borderline | 1/4 (25) | 0/3 (0) | 0/3 (0) | 0/2 (0) | 0/3 (0) | 1/4 (25) | | |
| Breast | 7/11 (64) | 6/16 (38)⁴ | 4/20 (20) | 3/8 (38) | 5/9 (56) | 3/14 (21) | 5/12 (42) | 4/13 (31) |
| Hepatocellular | 7/14 (50) | 6/13 (46) | 2/15 (13) | 8/15 (53) | 6/16 (38) | 4/13 (31) | | |
| Melanoma | 7/16 (44) | 2/11 (18) | 6/13 (46) | 4/15 (27) | 4/13 (31) | 4/15 (27) | | |
| Tongue | 4/7 (57) | 2/4 (50) | 0/7 (0) | N/A | 0/4 (0) | 0/9 (0) | | |
| Total³ | 47/103 (46) | 38/90 (42) | 29/96 (30) | 27/69 (39) | 28/87 (32) | 24/90 (27) | | |

¹Results are expressed as cases of allelic imbalance/informative cases with percentages in bold in brackets.
²Includes adenocarcinoma, germ cell tumors and tumors of mixed histology.
³Excludes benign and borderline ovarian tumors.
⁴Includes CEDD and 586F18b Al
N/A: not available

TABLE 2
Clinicopathological characteristics of
a serous ovarian cancer patient cohort

Parameter

| | |
|---|---|
| Age | 59.6 (26.4-86.4) |
| Grade of Disease | G1 = 7; G2 = 73; G3 = 80 (n = 160) |
| FIGO Stage | S1 = 10; S2 = 8; S3 = 121; S4 = 26 (n = 165) |
| Length of Follow up | 28.2 months (0-133.4) |
| Outcome | 101 died related to malignancy |
| | 5 died unrelated to malignancy |
| | 59 alive |

TABLE 3
Univariate analysis of clinicopathological parameters and EDD immunoreactivity to patient outcome

| | | Disease-specific survival | | Relapse-free survival | |
|---|---|---|---|---|---|
| Variable | Subgroup | Hazards Ratio (95% C.I.) | P | Hazards Ratio (95% C.I.) | P |
| Age | <65 vs ≧65 yrs | 1.069 (0.706-1.618) | 0.7538 | 0.889 (0.572-1.380 | 0.5991 |
| Stage | continuous | 1.642 (1.204-2.240) | 0.0017 | 1.447 (1.060-1.794) | 0.0198 |
| Grade | 1 vs 2, 3 | 4.393 (1.076-17.939) | 0.0392 | 2.726 (0.858-8.660) | 0.089 |
| CA-125 | <500 vs ≧500 | 1.101 (0.703-1.724) | 0.6738 | 1.271 (0.794-2.034) | 0.3173 |
| Performance status | 0, 1 vs 2, 3 | 3.999 (2.106-7.594) | <0.0001 | 1.360 (0.494-3.744) | 0.5511 |
| Residual Disease | <2 cm vs ≧2 cm | 2.974 (1.908-4.637) | <0.0001 | 1.942 (1.173-3.214) | 0.0098 |
| Menopausal status | Pre/peri vs post | 1.456 (0.870-2.439) | 0.1528 | 1.127 (0.677-1.874) | 0.6456 |
| EDD expression | EDD negative vs EDD positive | 1.411 (0.934-2.131) | 0.102 | 2.081 (1.328-3.262) | 0.0014 |

TABLE 4
Multivariate analysis of clinicopathological parameters
and EDD immunoreactivity to patient outcome.

| | | Hazards Ratio | |
|---|---|---|---|
| Variable | Subgroup | (95% C.I.) | P |
| Disease-specific Survival | | | |
| Stage | continuous | 1.635 | 0.0045 |
| | | (1.165-2.295) | |
| Grade | 1 vs 2, 3 | 3.745 | 0.0669 |
| | | (0.912-15.375) | |
| Performance status | 0, 1 vs 2, 3 | 2.692 | 0.0038 |
| | | (1.376-5.268) | |
| Residual Disease | <2 cm vs ≧2 cm | 2.149 | 0.0019 |
| | | (1.327-3.481) | |
| EDD expression | EDD negative | 1.374 | 0.1578 |
| | vs EDD positive | (0.884-2.135) | |
| Relapse-free Survival | | | |
| Stage | continuous | 1.494 | 0.0089 |
| | | (1.106-2.019) | |
| Residual Disease | <2 cm vs ≧2 cm | 1.674 | 0.0468 |
| | | (1.007-2.781) | |
| EDD expression | EDD negative | 2.301 | 0.0004 |
| | vs EDD positive | (1.454-3.643) | |

TABLE 5
Summary of EDD sequence variants detected in
26 normal epithelial and cancer cell lines.

| | | | Predicted | |
|---|---|---|---|---|
| Nucleotide | | | aa² | |
| positioⁿ | Codon | Base change | change | Cell line(s) |
| 4753 | 1584 | C→A | His→Asn | SK-BR-3 |
| 6279 | 2093 | A→G | Asn→Ser | IGROV-1 |
| 886-902 | 296-300 | Splice variant | VLLPL | Detected |
| | | | removed | in all cell |
| | | | | lines |
| 7689 | | A→C | No change | Hs 578T |
| 4055 | | C→T | No change | BT-20 |
| | | | | Human mRNA³ |
| 4390 | | A→G | No change | Human mRNA³ |
| 4556 | | A→G | No change | T-47D |
| | | | | MDA-MB-134 |
| 3956 | | A→G | No change | HMEC-184 |
| | | | | T-47D |
| | | | | MCF-7 |
| | | | | BT-549 |
| | | | | ZR-75-1 |
| | | | | MDA-MB-134 |
| | | | | DU-145 |
| | | | | LnCap |
| | | | | Hs 578T |
| 7634 | | C→A | No change | HMEC-184 |
| | | | | T-47D |
| | | | | MCF-7 |
| | | | | BT-549 |
| | | | | DU-145 |
| | | | | Hs 578T |

Base changes in bold confirmed at the genomic level.
¹Nucleotide numbering starts at the A of the initiation codon
²aa, amino acid
³David Anderson (unpublished data)

TABLE 6
Genotype analysis of embryos from Edd^{+/-}Intercrosses.
Observed and expected genotype frequencies (in brackets)
at various stages of gestation.

| | Age (days post coitum) | | | | |
|---|---|---|---|---|---|
| Genotype | 8.5 | 9.5 | 10.5 | 11.5 | 12.5 |
| Edd^{+/+} | 14(11) | 3(4) | 10(7) | 6(4) | 9(7) |
| Edd^{+/-} | 18(22) | 10(9) | 12(10) | 8(9) | 20(15) |
| Edd^{-/-} | 11(11) | 4(4) | 7(7) | 0(4) | 0(7) |

TABLE 7
Genotypic analysis of offspring from Edd^{+/Δ}/p53^{+/Δ}
double heterozygous intercrosses.

| | | AGE (days post coitum) | | |
|---|---|---|---|---|
| GENOTYPE | | 9.5 | 10.5 | 11.5 |
| p53^{+/+} | Edd^{+/+} | 1 | 2 | 3 |
| | Edd^{+/Δ} | 3 | 8 | 4 |
| | Edd^{Δ/Δ} | 4 | 0 | 0 |
| p53^{+/Δ} | Edd^{+/+} | 4 | 4 | 3 |
| | Edd^{+/Δ} | 4 | 12 | 11 |
| | Edd^{Δ/Δ} | 1 | 4 | 1 |
| p53^{Δ/Δ} | Edd^{+/+} | 2 | 4 | 2 |
| | Edd^{+/Δ} | 2 | 4 | 2 |
| | Edd^{Δ/Δ} | 1 | 2 | 6* |
| | Total | 22 | 40 | 32 |

*Embryos were partially resorbed and scored as non-viable.

TABLE 8
Gene Expression Changes in HMEC 184 and
MCF-7 Cells as a Result of EDD Silencing

| Gene expression | | |
|---|---|---|
| modulated in | Gene expression | Gene expression |
| HMEC 184 and | modulated in | modulated in |
| MCF-7 cells | HMEC 184 cells | and MCF-7 cells |
| R-ras (↑) | Coronin (↑) | Id-1 (↑) |
| WAVE-2 (↑) | Neuregulin1 (↑) | paxillin (↑) |
| AP1mu2 (↑) | Syndecan-4 (↑) | |
| Twinfilin (↑) | SFRP-1 (↓) | |
| Raf1 (↑) | FK506BP5 (↓) | |
| Cyclin D (↑)3 | SERPIN H1 (↓) | |
| RhoB (↑) | Collagen IVα1 (↓) | |
| ARP 2/3 complex (↑) | Wnt5a (↓) | |
| Core binding | | |
| factor beta (↑) | | |

(↑) indicates that gene expression is increased
(↓) indicates that gene expression is decreased

1. A method for detecting a cancer cell in a subject, said method comprising determining the level of nucleic acid that is linked to map position 8q22.3 of the human genome or an expression product thereof in a sample of said subject, wherein an elevated level of said nucleic acid or said polypeptide is indicative of cancer in the subject.

2. The method according to claim 1 wherein the cancer cell is epithelial in origin.

3. The method of claim 1 wherein the cancer cell is from a cancer selected from the group consisting of ovarian cancer, melanoma, metastatic melanoma, squamous cell carcinoma of the head and neck, squamous cell carcinoma of the tongue, hepatocellular carcinoma, breast cancer, a metastases of ovarian cancer, a metastases of melanoma, a metastases of metastatic melanoma, a metastases of squamous cell carcinoma of the head and neck, a metastases of squamous cell carcinoma of the tongue, a metastases of hepatocellular carcinoma and a metastases of breast cancer.

4. The method of claim 1 wherein the nucleic acid that is linked to map position 8q22.3 of the human genome comprises the genomic Edd and p53R2 genes or a portion thereof.

5. The method of claim 1 wherein the nucleic acid that is linked to map position 8q22.3 of the human genome comprises a genomic gene encoding an EDD protein.

6. The method of claim 5 wherein the EDD protein is a polypeptide that comprises an amino acid sequence having at least 80% identity to the sequence set forth in SEQ ID Nos: 2 or 4.

7. The method of claim 1, said method comprising:
(i) determining the level of nucleic acid linked to map position 8q22.3 of the human genome in a test sample from said subject; and
(ii) comparing the level of the nucleic acid at (i) to the level of the nucleic acid in a reference sample from a healthy or normal individual,
wherein a level of the nucleic acid at (ii) that is enhanced in the test sample relative to the reference sample from the normal or healthy individual is indicative of the presence of a cancer cell in said subject.

8. The method of claim 7 wherein the test sample and the reference sample comprise a cell from a tissue selected from the group consisting of skin, an oral cavity tissue, breast, liver, spleen, ovary, prostate, kidney, uterus, placenta, cervix, omentum, rectum, brain, bone, lung, lymph, urine, semen, blood, abdominal fluid, and serum.

9. The method of claim 1 wherein the level of nucleic acid linked to map position 8q22.3 of the human genome is determined by hybridizing a nucleic acid probe to genomic DNA encoding an EDD protein in the sample under stringency hybridization conditions and detecting the hybridization using a detection means.

10. The method of claim 9 wherein the detection means is nucleic acid hybridization or amplification reaction.

11. The method of claim 1 wherein the level of nucleic acid that is linked to map position 8q22.3 of the human genome is determined by hybridizing a nucleic acid probe or primer to genomic DNA and detecting the hybridization, wherein the probe or primer comprises a nucleotide sequence selected from the group consisting of:
(i) the sequence set forth in SEQ ID NO: 5;
(ii) the sequence set forth in SEQ ID NO: 6;
(iii) the sequence set forth in SEQ ID NO: 7;
(iv) the sequence set forth in SEQ ID NO: 24;
(v) the sequence set forth in SEQ ID NO: 25; and
(vi) the sequence of a nucleic acid fragment produced by amplification using any one of (i) to (v) as amplification primers in PCR.

12. The method of claim 1 wherein the sample has been obtained previously from the subject.

13. The method of claim 1 wherein the level of nucleic acid that is linked to map position 8q22.3 of the human genome is determined by hybridizing a nucleic acid probe or primer to genomic DNA and detecting the hybridization, wherein the probe or primer comprises a nucleotide sequence selected from the group consisting of:
(i) the sequence set forth in SEQ ID NO: 3;
(ii) the sequence set forth in SEQ ID NO: 5;
(iii) the sequence set forth in SEQ ID NO: 6;
(iv) the sequence set forth in SEQ ID NO: 7;
(v) the sequence set forth in SEQ ID NO: 24;
(vi) the sequence set forth in SEQ ID NO: 25;
(vii) the sequence of a nucleic acid fragment produced by amplification using (v) and (vi) as amplification primers in PCR;
(viii) the sequence set forth in SEQ ID NO:26;
(ix) the sequence set forth in SEQ ID NO: 27;
(x) the sequence of a nucleic acid fragment produced by amplification using (viii) and (ix) as amplification primers in PCR;
(xi) the sequence set forth in SEQ ID NO: 28;
(xii) the sequence set forth in SEQ ID NO: 29;
(xiii) the sequence set forth in SEQ ID NO: 30;
(xiv) the sequence of a nucleic acid fragment produced by amplification using (xii) and (xiii) as amplification primers in PCR;
(xv) the sequence set forth in SEQ ID NO: 33;
(xvi) the sequence set forth in SEQ ID NO: 34;
(xvii) the sequence of a nucleic acid fragment produced by amplification using (xv) and (xvi) as amplification primers in PCR;
(xviii) the sequence set forth in SEQ ID NO: 37;
(xix) the sequence set forth in SEQ ID NO: 38;
(xx) the sequence of a nucleic acid fragment produced by amplification using (xvii) and (xix) as amplification primers in PCR;
(xxi) the sequence set forth in SEQ ID NO: 40; and
(xxii) a sequence that is complementary to any one of (i) to (xxi).

14. A method for detecting a cancer cell in a subject, said method comprising:
(i) determining the level of mRNA encoded by nucleic acid linked to map position 8q22.3 of the human genome that is expressed in a test sample from said subject; and
(ii) comparing the level of the mRNA determined at (i) to the level of mRNA encoded by nucleic acid linked to map position 8q22.3 of the human genome that is expressed in a reference sample from a healthy or normal individual,
wherein a level of the mRNA at (i) that is enhanced in the test sample relative to the reference sample from the normal or healthy individual is indicative of the presence of a cancer cell in said subject.

15-18. (canceled)

19. A method for diagnosing a cancer or predicting recurrence of a cancer in a subject comprising determining the level of mRNA or protein encoded by nucleic acid linked to map position 8q22.3 of the human genome in a sample of said subject, wherein an elevated level of said mRNA or protein is indicative of relapse of a cancer in said subject.

20-25. (canceled)

26. An isolated nucleic acid molecule for detecting a cancer cell comprising a nucleotide sequence selected from the group consisting of:
(i) a sequence that encodes the amino acid sequence set forth in SEQ ID NO: 4 wherein said amino acid sequence lacks the sequence VLLLPL;
(ii) the sequence set forth in SEQ ID NO: 3;
(iii) the sequence set forth in SEQ ID NO: 5;
(iv) the sequence set forth in SEQ ID NO: 6;
(v) the sequence set forth in SEQ ID NO: 7;
(vi) the sequence set forth in SEQ ID NO: 24;
(vii) the sequence set forth in SEQ ID NO: 25;
(viii) the sequence of a nucleic acid fragment produced by amplification using (vi) and (vii) as amplification primers in PCR;
(ix) the sequence set forth in SEQ ID NO:26;
(x) the sequence set forth in SEQ ID NO: 27;
(xi) the sequence of a nucleic acid fragment produced by amplification using (ix) and (x) as amplification primers in PCR;
(xii) the sequence set forth in SEQ ID NO: 28;
(xiii) the sequence set forth in SEQ ID NO: 29;
(xiv) the sequence set forth in SEQ ID NO: 30;
(xv) the sequence of a nucleic acid fragment produced by amplification using (xiii) and (xiv) as amplification primers in PCR;
(xvi) the sequence set forth in SEQ ID NO: 33;
(xvii) the sequence set forth in SEQ ID NO: 34;
(xviii) the sequence of a nucleic acid fragment produced by amplification using (xvi) and (xvii) as amplification primers in PCR;
(xix) the sequence set forth in SEQ ID NO: 37;
(xx) the sequence set forth in SEQ ID NO: 38;
(xxi) the sequence of a nucleic acid fragment produced by amplification using (xix) and (xx) as amplification primers in PCR;
(xxii) the sequence set forth in SEQ ID NO: 40; and
(xxiii) a sequence that is complementary to any one of (i) to (xxi).

27. An isolated or recombinant protein complex comprising:
(i) an EDD protein or a portion of an EDD protein sufficient to bind to a protein selected from the group consisting of a protein having tumor suppressor activity, a protein having cell cycle modulatory activity, a protein associated with DNA repair or damage, a nuclear targeting protein, and a progesterone receptor protein; and
(ii) a nuclear protein selected from the group consisting of a protein having tumor suppressor activity, a protein having cell cycle modulatory activity, a protein associated with DNA repair or damage, a nuclear targeting protein and a progesterone receptor protein or a portion of said protein sufficient to bind to said EDD protein or said portion of an EDD protein.

28-36. (canceled)

37. An isolated antibody that binds to a protein complex comprising an EDD protein.

38-41. (canceled)

42. An isolated antibody that binds to the antibody of claim 3.

43. A kit for detecting or producing a protein complex, said kit comprising an EDD polypeptide or a portion of an EDD polypeptide and a second polypeptides selected from the group consisting of a protein having tumor suppressor activity, a protein having cell cycle modulatory activity, a protein associated with DNA repair or damage, a nuclear targeting protein, and a progesterone receptor protein or a portion thereof, wherein the portion of the second polypeptide is sufficient to bind to said EDD polypeptide or said portion of an EDD polypeptide.

44-46. (canceled)

47. A kit for detecting or producing a protein complex comprising:
(i) a first compartment comprising an EDD protein or a portion thereof sufficient to form a protein complex selected from the group consisting of: a protein having tumor suppressor activity, a protein having cell cycle modulatory activity, a protein associated with DNA repair or damage, a nuclear targeting protein, and a progesterone receptor protein or a portion thereof, wherein the portion of the second polypeptide is sufficient to bind to said EDD polypeptide or said portion of an EDD polypeptide; and
(ii) a second compartment comprising an antibody or ligand that binds to a protein selected from the group consisting of a protein having tumor suppressor activity, a protein having cell cycle modulatory activity, a protein associated with DNA repair or damage, a nuclear targeting protein, and a progesterone receptor protein or a portion thereof.
wherein said antibody or ligand that binds to a protein complex does not bind to the individual protein binding partners.

48. A kit for detecting or producing a protein complex comprising:
(i) a first compartment comprising an EDD protein or a portion thereof sufficient to form a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor; and
(ii) a second compartment comprising an antibody or ligand that binds to a protein selected from the group consisting of (i) a CHK2 protein; (ii) a BRCA2 protein; (iii) a CIBCIB protein; (iv) an importin alpha-1 protein; (v) an importin alpha-3 protein; (vi) an importin alpha-5 protein; and (vii) a progesterone receptor protein, or an antibody or ligand that binds to a protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor.
wherein said antibody or ligand that binds to a protein complex does not bind to the individual protein binding partners.

49. A kit for detecting or producing a protein complex comprising:
(i) a first compartment comprising an antibody or ligand that binds to an EDD protein; and
(ii) a second compartment comprising a protein selected from the group consisting of (i) a CHK2 protein; (ii) a BRCA2 protein; (iii) a CIB protein; (iv) an importin alpha-1 protein; (v) an importin alpha-3 protein; (vi) an importin alpha-5 protein; and (vii) a progesterone receptor protein, or a portion thereof sufficient to bind to an EDD protein.

50. A kit for detecting or producing a protein complex comprising:
(i) a first compartment comprising an isolated or recombinant protein complex selected from the group consisting of: (i) a complex comprising EDD and CHK2; (ii) a complex comprising EDD and BRCA2; (iii) a complex comprising EDD and CIB; (iv) a complex comprising EDD and importin alpha-1; (v) a complex comprising EDD and importin alpha-3; (vi) a complex comprising EDD and importin alpha-5; and (vii) a complex comprising EDD and progesterone receptor; and
(ii) a second compartment comprising an (i) antibody or ligand that binds to a polypeptide selected from the group consisting of a CHK2 protein, a BRCA2 protein, a CIB protein, an importin alpha-1 protein, an importin alpha-3 protein, an importin alpha-5 protein, a progesterone receptor protein and an EDD protein; or (ii) an antibody or ligand that binds to one or more protein complexes (a).

51. A method for isolating a protein complex comprising an EDD protein or a protein of an EDD protein and a protein selected from the group consisting of a protein having tumor suppressor activity, a protein having cell cycle modulatory activity, a protein associated with DNA repair or damage, a nuclear targeting protein, and a progesterone receptor protein from a suitable cellular source, said method comprising contacting an extract of said cell with an EDD polypeptide or a portion thereof that binds to said protein for a time and under conditions sufficient for a protein complex to form and then isolating the protein complex formed.

52. A method of isolating the protein complex comprising an EDD protein or a protein of an EDD protein and a protein selected from the group consisting of a protein having tumor suppressor activity, a protein having cell cycle modulatory activity, a protein associated with DNA repair or damage, a nuclear targeting protein, and a progesterone receptor protein comprising:
(i) isolating a protein that is a component of the protein complex from a cell that expresses said protein;
(ii) isolating another protein that is a component of the protein complex from a cell that expresses said other protein; and
combining the proteins isolated at (i) and (ii) in an amount and under conditions sufficient to facilitate the formation of a protein complex.

53-54. (canceled)

55. A method for determining a predisposition for disease, or a disease state, said method comprising detecting a protein complex comprising:
(i) an EDD protein; and
(ii) a protein selected from the group consisting of a protein having tumor suppressor activity, a protein having cell cycle modulatory activity, a protein associated with DNA repair or damage, a nuclear targeting protein, a progesterone receptor protein and a protein associated with vascularization,
wherein an elevated level of said protein complex is indicative of a predisposition for disease, or a disease state in said subject.

56-57. (canceled)

58. A method for determining a modulator of the activity, formation or stability of an isolated or recombinant protein complex comprising:
(i) determining the activity, formation or stability of a protein complex comprising (a) an EDD protein or a portion of an EDD protein; and (b) a protein selected from the group consisting of a protein having tumor suppressor activity, a protein having cell cycle modulatory activity, a protein associated with DNA repair or damage, a nuclear targeting protein, and a progesterone receptor protein or a portion thereof, wherein the portion of the second polypeptide is sufficient to bind to said EDD polypeptide or said portion of an EDD polypeptide, in the absence of a candidate compound or candidate antibody; and
(ii) determining the level of said protein complex in the presence of a candidate compound or in the presence of said candidate antibody
wherein a difference in the level of said protein complex at (i) and (ii) indicates that the candidate compound or candidate antibody is a modulator of said interaction.

59. A method for determining a modulator of the level of protein complex formation comprising:
(i) determining the level of a protein complex comprising (a) an EDD protein or a portion of an EDD protein; and (b) a protein selected from the group consisting of a protein having tumor suppressor activity, a protein having cell cycle modulatory activity, a protein associated with DNA repair or damage, a nuclear targeting protein, and a progesterone receptor protein or a portion thereof, wherein the portion of the second polypeptide is sufficient to bind to said EDD polypeptide or said portion of an EDD polypeptide, in the absence of a candidate compound or candidate antibody; and
(ii) determining the level of said protein complex in the presence of a candidate compound or in the presence of said candidate antibody
wherein a difference in the level of said protein complex at (i) and (ii) indicates that the candidate compound or candidate antibody is a modulator of said interaction.

60-62. (canceled)

63. A method for treating a condition associated with elevated expression of an EDD protein in a cell, said method comprising administering an amount of a compound effective to reduce EDD expression in a cell.

64-68. (canceled)

69. An antisense nucleic acid, ribozyme, PNA, interfering RNA or siRNA comprising a sequence having at least 80% homology to SEQ ID NO: 47.

70-71. (canceled)

72. A pharmaceutical composition comprising the antisense nucleic acid, ribozyme, PNA, interfering RNA or siRNA of claim 69.

73-74. (canceled)

75. A method for determining the ability of a cell to phosphorylate CHK2 in response to a DNA damaging agent comprising determining the level of expression of EDD in said cell, wherein reduced or suppressed EDD expression indicates that the cell has reduced ability to phosphorylate CHK2 in response to a DNA damaging agent.


SEQUENCE LISTING (53 sequences)

SEQ ID NO: 1 - DNA, 8509 bp, human Edd
cgccctcgagtggaggacgagaaggaaagcaccatgacgtccatccatttcgtggttcacccgctgccgggcaccgaggaccagctcaatgacaggttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttaggaagccgctggagttctggagtgggtggaagtggtggaggatcctctggtaggtcatcagctggagctcgagattcccgccggcagactcgagttattcggacaggacgggatcgagggtctgggcttttgggcagtcagccccagccagttattccagcatctgtcattccagaggagctgatttcacaggcccaagttgttttacaaggcaaatccagaagtgtcattattcgagaacttcagagaacaaatcttgatgtgaaccttgctgtaaataatttacttagccgggatgatgaagatggagatgatggggatgatacagccagcgaatcttatttgcctggagaggatcttatgtctctccttgatgccgacattcattctgcccacccaagtgtcattattgatgcagatgccatgttttctgaagacattagctattttggttacccttcttttcgtcgttcatcactttccaggctaggctcatctcgagttctccttcttcccttagagagagactctgagctgttgcgtgaacgtgaatccgttttacgtttacgtgaacgaaggtggcttgatggagcctcatttgataatgaaaggggttctaccagcaaggaaggagagccaaacttggataagaagaatacacctgttcaaagtccagtatctctaggagaagatttgcagtggtggcctgataaggatggaacaaaattcatctgtattggggctctgtattctgaacttctggctgtcagcagtaaaggagaactttatcagtggaaatggagtgaatctgagccttacagaaatgcccagaatccttcattacatcatccacgagcaacatttttggggttaaccaatgaaaagatagtcctcctgtctgcaaatagcataagagcaactgtagctacagaaaataacaaggttgctacatgggtggatgaaactttaagttctgtggcttctaaattagagcacactgctcagacttactctgaacttcaaggagagcggatagtttctttacattgctgtgccctttacacctgcgctcagctggaaaacagtttatattggtggggtgtagttccttttagtcaaaggaagaaaatgttagagaaagctagagcaaaaaataaaaagcctaaatccagtgctggtatttcttcaatgccgaacatcactgttggtacccaggtatgcttgagaaataatcctctttatcatgctggagcagttgcattttcaattagtgctgggattcctaaagttggtgtcttaatggagtcagtttggaatatgaatgacagctgtagatttcaacttagatctcctgaaagcttgaaaaacatggaaaaagctagcaaaactactgaagctaagcctgaaagtaagcaggagccagtgaaaacagaaatgggtcctccaccatctccagcatccacgtgtagtgatgcatcctcaattgccagcagtgcatcaatgccatacaaacgacgacggtcaacccctgcaccaaaagaagaggaaaaggtgaatgaagagcagtggtctcttcgggaagtggtttttgtggaagatgtcaagaatgttcctgttggcaaggtgctaaaagtagatggtgcctatgttgctgtaaaatttccaggaacctccagtaatactaactgtcagaacagctctggtccagatgctgacccttcttctctcctgcaggattgtaggttacttagaattgatgaattgcaggttgtcaaaactggtggaacaccgaaggttcccgactgtttccaaaggactcctaaaaagctttgtatacctgaaaaaacagaaatattagcagtgaatgtagattccaaaggtgttcatgctgttctgaagactggaaattgggtgcgatactgtatctttgatcttgctacaggaaaagcagaacaggaaaataattttcctacaagcagcattgctttccttggtcagaatgagaggaatgtagccattttcactgctggacaggaatctcccattattcttcgagatggaaatggtaccatctacccaatggccaaagattgcatgggaggaataagggatcccgattggctggatcttccacctattagtagtcttggaatgggtgtgcattctttaataaatcttcctgccaattcaacaatcaaaaagaaagctgctgttatcatcatggctgtagagaaacaaaccttaatgcaacacattctgcgctgtgactatgaggcctgtcgacaatatctaatgaatcttgagcaagcggttgttttagagcagaatctacagatgctgcagacattcatcagccacagatgtgatggaaatcgaaatattttgcatgcttgtgtatcagtttgctttccaaccagcaataaagaaactaaagaagaagaggaagcggagcgttctgaaagaaatacatttgcagaaaggctttctgctgttgaggccattgcaaatgcaatatcagttgtttcaagtaatggcccaggtaatcgggctggatcatcaagtagccgaagtttgagattacgggaaatgatgagacgttcgttgagagcagctggtttgggtagacatgaagctggagcttcatccagtgaccaccaggatccagtttcaccccccatagctccccctagttgggttcctgaccctcctgcgatggatcctgatggtgacattgattttatcctggcccccgctgtgggatctcttaccacagcagcaaccggtactggtcaaggaccaagcacctccactattccaggtccttccacagagccatctgtagtagaatccaaggatcgaaaggcgaatgctcattttatattgaaattgttatgtgacagtgtggttctccagccctatctacgagaacttctttctgccaaggatgcaagagggatgaccccatttatgtcagctgtaagtggccgagcttatcctgctgcaattaccatcttagaaactgctcagaaaattgcaaaagctgaaatatcctcaagtgaaaaagaggaagatgtattcatgggaatggtttgcccatcaggtaccaaccctgatgactctcctttatatgttttatgttgtaatgacacttgcagttttacatggactggagcagagcacattaaccaggatatttttgagtgtcgaacttgtggcttgctggagtcactgtgttgttgtacggaatgtgcaagggtttgtcataaaggtcatgattgcaaactcaaacggacatcaccaacagcctactgtgattgttgggagaaatgtaaatgtaaaactcttattgctggacagaaatctgctcgtcttgatctactttatcgcctgctcactgctactaatctggttactctgccaaacagcaggggagagcacctcttactattcttagtacagacagtcgcaaggcagacggtggagcattgtcaatacaggccacctcgaatcagggaagatcgtaaccgaaaaacagccagtcctgaagattcagatatgccagatcatgatttagagcctccaagatttgcccagcttgcattggagcgtgttctacaggactggaatgccttgaaatctatgattatgtttgggtcgcaggagaataaagaccctcttagtgccagcagtagaataggccatcttttgccagaagagcaagtatacctcaatcagcaaagtggcacaattcggctggactgtttcactcattgccttatagttaagtgtacagcagatattttgcttttagatactctactaggtacactagtgaaagaactccaaaacaaatatacacctggacgtagagaagaagctattgctgtgacaatgaggtttctacgttcagtggcaagagtttttgttattctgagtgtggaaatggcttcatccaaaaagaaaaacaactttattccacagccaattggaaaatgcaagcgtgtattccaagcattgctaccttacgctgtggaagaattgtgcaacgtagcagagtcactgattgttcctgtcagaatggggattgctcgtccaactgcaccatttaccctggctagtactagcatagatgccatgcagggcagtgaagaattattttcagtggaaccactaccaccacgaccatcatctgatcagtctagcagctccagtcagtctcagtcatcctacatcatcaggaatccacagcagaggcgcatcagccagtcacagcccgttcggggcagagatgaagaacaggatgatattgtttcagcagatgtggaagaggttgaggtggtggagggtgtggctggagaagaggatcatcatgatgaacaggaagaacacggggaagaaaatgctgaggcagagggacaacatgatgagcatgatgaagacgggagtgatatggagctggacttgttagcagcagctgaaacagaaagtgatagtgaaagtaaccacagcaaccaagataatgctagtgggcgcagaagcgttgtcactgcagcaactgctggttcagaagcaggagcaagcagtgttcctgccttcttttctgaagatgattctcaatcgaatgactcaagtgattctgatagcagtagtagtcagagtgacgacatagaacaggagacctttatgcttgatgagccattagaaagaaccacaaatagctcccatgccaatggtgctgcccaagctccccgttcaatgcagtgggctgtccgcaacacccagcatcagcgagcagccagtacagccccttccagtacatctacaccagcagcaagttcagcgggtttgatttatattgatccttcaaacttacgccggagtggtaccatcagtacaagtgctgcagctgcagcagctgctttggaagctagcaacgccagcagttacctaacatctgcaagcagtttagccagggcttacagcattgtcattagacaaatctcggacttgatgggccttattcctaagtataatcacctagtatactctcagattccagcagctgtgaaattgacttaccaagatgcagtaaacttacagaactatgtagaagaaaagcttattcccacttggaactggatggtcagtattatggattctactgaagctcaattacgttatggttctgcattagcatctgctggtgatcctggacatccaaatcatcctcttcacgcttctcagaattcagcgagaagagagaggatgactgcgcgagaagaagctagcttacgaacacttgaaggcagacgacgtgccaccttgcttagcgcccgtcaaggaatgatgtctgcacgaggagacttcctaaattatgctctgtctctaatgcggtctcataatgatgagcattctgatgttcttccagttttggatgtttgctcattgaagcatgtggcatatgtttttcaagcacttatatactggattaaggcaatgaatcagcagacaacattggatacacctcaactagaacgcaaaaggacgcgagaactcttggaactgggtattgataatgaagattcagaacatgaaaatgatgatgacaccaatcaaagtgctactttgaatgataaggatgatgactctcttcctgcagaaactggccaaaaccatccatttttccgacgttcagactccatgacattccttgggtgtatacccccaaatccatttgaagtgcctctggctgaagccatccccttggctgatcagccacatctgttgcagccaaatgctagaaaggaggatctttttggccgtccaagtcagggtctttattcttcatctgccagtagtgggaaatgtttaatggaggttacagtggatagaaactgcctagaggttcttccaacaaaaatgtcttatgctgccaatctgaaaaatgtaatgaacatgcaaaaccggcaaaaaaaagaaggggaagaacagcccgtgctgccagaagaaactgagagttcaaaaccagggccatctgctcatgatcttgctgcacaattaaaaagtagcttactagcagaaataggacttactgaaagtgaagggccacctctcacatctttcaggccacagtgtagctttatgggaatggttatttcccatgatatgctgctaggacgttggcgcctttctttagaactgttcggcagggtattcatggaagatgttggagcagaacctggatcaatcctaactgaattgggtggttttgaggtaaaagaatcaaaattccgcagagaaatggaaaaactgagaaaccagcagtcaagagatttgtcactagaggttgatcgggatcgagatcttctcattcagcagactatgaggcagcttaacaatcactttggtcgaagatgtgctactacaccaatggctgtacacagagtaaaagtcacatttaaggatgagccaggagagggcagtggtgtagcacgaagtttttatacagccattgcacaagcatttttatcaaatgaaaaattgccaaatctagagtgtatccaaaatgccaacaaaggcacccacacaagtttaatgcagagattaaggaaccgaggagagagagaccgggaaagggagagagaaagggaaatgaggaggagtagtggtttgcgagcaggttctcggagggaccgggatagagactttagaagacagctttccatcgacactaggccctttagaccagcctctgaagggaatcctagcgatgatcctgagcctttgccagcacatcggcaggcacttggagagaggctttatcctcgtgtacaagcaatgcaaccagcatttgcaagtaaaatcactggcatgttgttggaattatccccagctcagctgcttctccttctagcaagtgaggattctctgagagcaagagtggatgaggccatggaactcattattgcacatggacgggaaaatggagctgatagtatcctggatcttggattagtagactcctcagaaaaggtacagcaggaaaaccgaaagcgccatggctctagtcgaagtgtagtagatatggatttagatgatacagatgatggtgatgacaatgcccctttgttttaccaacctgggaaaagaggattttatactccaaggcctggcaagaacacagaagcaaggttgaattgtttcagaaacattggcaggattcttggactatgtctgttacagaatgaactctgtcctatcacattgaatagacatgtaattaaagtattgcttggtagaaaagtcaattggcatgattttgctttttttgatcctgtaatgtatgagagtttgcggcaactaatcctcgcgtctcagagttcagatgctgatgctgttttctcagcaatggatttggcatttgcaattgacctgtgtaaagaagaaggtggaggacaggttgaactcattcctaatggtgtaaatataccagtcactccacagaatgtatatgagtatgtgcggaaatacgcagaacacagaatgttggtagttgcagaacagcccttacatgcaatgaggaaaggtctactagatgtgcttccaaaaaattcattagaagatttaacggcagaagattttaggcttttggtaaatggctgcggtgaagtcaatgtgcaaatgctgatcagttttacctctttcaatgatgaatcaggagaaaatgctgagaagcttctgcagttcaagcgttggttctggtcaatagtagagaagatgagcatgacagaacgacaagatcttgtttacttttggacatcaagcccatcactgccagccagtgaagaaggattccagcctatgccctcaatcacaataagaccaccagatgaccaacatcttcctactgcaaatacttgcatttctcgactttacgtcccactctattcctctaaacagattctcaaacagaaattgttactcgccattaagaccaagaattttggttttgtgtagagtataaaaagtgtgtattgctgtgtaatattactagcaaattttgtagatttttttccatttgtctataaaagtt

SEQ ID NO: 2 - protein, 2799 aa, human EDD protein
MTSIHFVVHPLPGTEDQLNDRLREVSEKLNKYNLNSHPPLNVLEQATIKQCVVGPNHAAFLLEDGRVCRIGFSVQPDRLELGKPDNNDGSKLNSNSGAGRTSRPGRTSDSPWFLSGSETLGRLAGNTLGSRWSSGVGGSGGGSSGRSSAGARDSRRQTRVIRTGRDRGSGLLGSQPQPVIPASVIPEELISQAQVVLQGKSRSVIIRELQRTNLDVNLAVNNLLSRDDEDGDDGDDTASESYLPGEDLMSLLDADIHSAHPSVIIDADAMFSEDISYFGYPSFRRSSLSRLGSSRVLLLPLERDSELLRERESVLRLRERRWLDGASFDNERGSTSKEGEPNLDKKNTPVQSPVSLGEDLQWWPDKDGTKFICIGALYSELLAVSSKGELYQWKWSESEPYRNAQNPSLHHPRATFLGLTNEKIVLLSANSIRATVATENNKVATWVDETLSSVASKLEHTAQTYSELQGERIVSLHCCALYTCAQLENSLYWWGVVPFSQRKKMLEKARAKNKKPKSSAGISSMPNITVGTQVCLRNNPLYHAGAVAFSISAGIPKVGVLMESVWNMNDSCRFQLRSPESLKNMEKASKTTEAKPESKQEPVKTEMGPPPSPASTCSDASSIASSASMPYKRRRSTPAPKEEEKVNEEQWSLREVVFVEDVKNVPVGKVLKVDGAYVAVKFPGTSSNTNCQNSSGPDADPSSLLQDCRLLRIDELQVVKTGGTPKVPDCFQRTPKKLCIPEKTEILAVNVDSKGVHAVLKTGNWVRYCIFDLATGKAEQENNFPTSSIAFLGQNERNVAIFTAGQESPIILRDGNGTIYPMAKDCMGGIRDPDWLDLPPISSLGMGVHSLINLPANSTIKKKAAVIIMAVEKQTLMQHILRCDYEACRQYLMNLEQAVVLEQNLQMLQTFISHRCDGNRNILHACVSVCFPTSNKETKEEEEAERSERNTFAERLSAVEAIANAISVVSSNGPGNRAGSSSSRSLRLREMMRRSLRAAGLGRHEAGASSSDHQDPVSPPIAPPSWVPDPPAMDPDGDIDFILAPAVGSLTTAATGTGQGPSTSTIPGPSTEPSVVESKDRKANAHFILKLLCDSVVLQPYLRELLSAKDARGMTPFMSAVSGRAYPAAITILETAQKIAKAEISSSEKEEDVFMGMVCPSGTNPDDSPLYVLCCNDTCSFTWTGAEHINQDIFECRTCGLLESLCCCTECARVCHKGHDCKLKRTSPTAYCDCWEKCKCKTLIAGQKSARLDLLYRLLTATNLVTLPNSRGEHLLLFLVQTVARQTVEHCQYRPPRIREDRNRKTASPEDSDMPDHDLEPPRFAQLALERVLQDWNALKSMIMFGSQENKDPLSASSRIGHLLPEEQVYLNQQSGTIRLDCFTHCLIVKCTADILLLDTLLGTLVKELQNKYTPGRREEAIAVTMRFLRSVARVFVILSVEMASSKKKNNFIPQPIGKCKRVFQALLPYAVEELCNVAESLIVPVRMGIARPTAPFTLASTSIDAMQGSEELFSVEPLPPRPSSDQSSSSSQSQSSYIIRNPQQRRISQSQPVRGRDEEQDDIVSADVEEVEVVEGVAGEEDHHDEQEEHGEENAEAEGQHDEHDEDGSDMELDLLAAAETESDSESNHSNQDNASGRRSVVTAATAGSEAGASSVPAFFSEDDSQSNDSSDSDSSSSQSDDIEQETFMLDEPLERTTNSSHANGAAQAPRSMQWAVRNTQHQRAASTAPSSTSTPAASSAGLIYIDPSNLRRSGTISTSAAAAAAALEASNASSYLTSASSLARAYSIVIRQISDLMGLIPKYNHLVYSQIPAAVKLTYQDAVNLQNYVEEKLIPTWNWMVSIMDSTEAQLRYGSALASAGDPGHPNHPLHASQNSARRERMTAREEASLRTLEGRRRATLLSARQGMMSARGDFLNYALSLMRSHNDEHSDVLPVLDVCSLKHVAYVFQALIYWIKAMNQQTTLDTPQLERKRTRELLELGIDNEDSEHENDDDTNQSATLNDKDDDSLPAETGQNHPFFRRSDSMTFLGCIPPNPFEVPLAEAIPLADQPHLLQPNARKEDLFGRPSQGLYSSSASSGKCLMEVTVDRNCLEVLPTKMSYAANLKNVMNMQNRQKKEGEEQPVLPEETESSKPGPSAHDLAAQLKSSLLAEIGLTESEGPPLTSFRPQCSFMGMVISHDMLLGRWRLSLELFGRVFMEDVGAEPGSILTELGGFEVKESKFRREMEKLRNQQSRDLSLEVDRDRDLLIQQTMRQLNNHFGRRCATTPMAVHRVKVTFKDEPGEGSGVARSFYTAIAQAFLSNEKLPNLECIQNANKGTHTSLMQRLRNRGERDREREREREMRRSSGLRAGSRRDRDRDFRRQLSIDTRPFRPASEGNPSDDPEPLPAHRQALGERLYPRVQAMQPAFASKITGMLLELSPAQLLLLLASEDSLRARVDEAMELIIAHGRENGADSILDLGLVDSSEKVQQENRKRHGSSRSVVDMDLDDTDDGDDNAPLFYQPGKRGFYTPRPGKNTEARLNCFRNIGRILGLCLLQNELCPITLNRHVIKVLLGRKVNWHDFAFFDPVMYESLRQLILASQSSDADAVFSAMDLAFAIDLCKEEGGGQVELIPNGVNIPVTPQNVYEYVRKYAEHRMLVVAEQPLHAMRKGLLDVLPKNSLEDLTAEDFRLLVNGCGEVNVQMLISFTSFNDESGENAEKLLQFKRWFWSIVEKMSMTERQDLVYFWTSSPSLPASEEGFQPMPSITIRPPDDQHLPTANTCISRLYVPLYSSKQILKQKLLLAIKTKNFGFV

SEQ ID NO: 3 - DNA, 8491 bp, human Edd cDNA splice variant
  CDS (34)..(8412)
cgccctcgagtggaggacgagaaggaaagcaccatgacgtccatccatttcgtggttcacccgctgccgggcaccgaggaccagctcaatgacaggttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttaggaagccgctggagttctggagtgggtggaagtggtggaggatcctctggtaggtcatcagctggagctcgagattcccgccggcagactcgagttattcggacaggacgggatcgagggtctgggcttttgggcagtcagccccagccagttattccagcatctgtcattccagaggagctgatttcacaggcccaagttgttttacaaggcaaatccagaagtgtcattattcgagaacttcagagaacaaatcttgatgtgaaccttgctgtaaataatttacttagccgggatgatgaagatggagatgatggggatgatacagccagcgaatcttatttgcctggagaggatcttatgtctctccttgatgccgacattcattctgcccacccaagtgtcattattgatgcagatgccatgttttctgaagacattagctattttggttacccttcttttcgtcgttcatcactttccaggctaggctcatctcgagagagagactctgagctgttgcgtgaacgtgaatccgttttacgtttacgtgaacgaaggtggcttgatggagcctcatttgataatgaaaggggttctaccagcaaggaaggagagccaaacttggataagaagaatacacctgttcaaagtccagtatctctaggagaagatttgcagtggtggcctgataaggatggaacaaaattcatctgtattggggctctgtattctgaacttctggctgtcagcagtaaaggagaactttatcagtggaaatggagtgaatctgagccttacagaaatgcccagaatccttcattacatcatccacgagcaacatttttggggttaaccaatgaaaagatagtcctcctgtctgcaaatagcataagagcaactgtagctacagaaaataacaaggttgctacatgggtggatgaaactttaagttctgtggcttctaaattagagcacactgctcagacttactctgaacttcaaggagagcggatagtttctttacattgctgtgccctttacacctgcgctcagctggaaaacagtttatattggtggggtgtagttccttttagtcaaaggaagaaaatgttagagaaagctagagcaaaaaataaaaagcctaaatccagtgctggtatttcttcaatgccgaacatcactgttggtacccaggtatgcttgagaaataatcctctttatcatgctggagcagttgcattttcaattagtgctgggattcctaaagttggtgtcttaatggagtcagtttggaatatgaatgacagctgtagatttcaacttagatctcctgaaagcttgaaaaacatggaaaaagctagcaaaactactgaagctaagcctgaaagtaagcaggagccagtgaaaacagaaatgggtcctccaccatctccagcatccacgtgtagtgatgcatcctcaattgccagcagtgcatcaatgccatacaaacgacgacggtcaacccctgcaccaaaagaagaggaaaaggtgaatgaagagcagtggtctcttcgggaagtggtttttgtggaagatgtcaagaatgttcctgttggcaaggtgctaaaagtagatggtgcctatgttgctgtaaaatttccaggaacctccagtaatactaactgtcagaacagctctggtccagatgctgacccttcttctctcctgcaggattgtaggttacttagaattgatgaattgcaggttgtcaaaactggtggaacaccgaaggttcccgactgtttccaaaggactcctaaaaagctttgtatacctgaaaaaacagaaatattagcagtgaatgtagattccaaaggtgttcatgctgttctgaagactggaaattgggtgcgatactgtatctttgatcttgctacaggaaaagcagaacaggaaaataattttcctacaagcagcattgctttccttggtcagaatgagaggaatgtagccattttcactgctggacaggaatctcccattattcttcgagatggaaatggtaccatctacccaatggccaaagattgcatgggaggaataagggatcccgattggctggatcttccacctattagtagtcttggaatgggtgtgcattctttaataaatcttcctgccaattcaacaatcaaaaagaaagctgctgttatcatcatggctgtagagaaacaaaccttaatgcaacacattctgcgctgtgactatgaggcctgtcgacaatatctaatgaatcttgagcaagcggttgttttagagcagaatctacagatgctgcagacattcatcagccacagatgtgatggaaatcgaaatattttgcatgcttgtgtatcagtttgctttccaaccagcaataaagaaactaaagaagaagaggaagcggagcgttctgaaagaaatacatttgcagaaaggctttctgctgttgaggccattgcaaatgcaatatcagttgtttcaagtaatggcccaggtaatcgggctggatcatcaagtagccgaagtttgagattacgggaaatgatgagacgttcgttgagagcagctggtttgggtagacatgaagctggagcttcatccagtgaccaccaggatccagtttcaccccccatagctccccctagttgggttcctgaccctcctgcgatggatcctgatggtgacattgattttatcctggcccccgctgtgggatctcttaccacagcagcaaccggtactggtcaaggaccaagcacctccactattccaggtccttccacagagccatctgtagtagaatccaaggatcgaaaggcgaatgctcattttatattgaaattgttatgtgacagtgtggttctccagccctatctacgagaacttctttctgccaaggatgcaagagggatgaccccatttatgtcagctgtaagtggccgagcttatcctgctgcaattaccatcttagaaactgctcagaaaattgcaaaagctgaaatatcctcaagtgaaaaagaggaagatgtattcatgggaatggtttgcccatcaggtaccaaccctgatgactctcctttatatgttttatgttgtaatgacacttgcagttttacatggactggagcagagcacattaaccaggatatttttgagtgtcgaacttgtggcttgctggagtcactgtgttgttgtacggaatgtgcaagggtttgtcataaaggtcatgattgcaaactcaaacggacatcaccaacagcctactgtgattgttgggagaaatgtaaatgtaaaactcttattgctggacagaaatctgctcgtcttgatctactttatcgcctgctcactgctactaatctggttactctgccaaacagcaggggagagcacctcttactattcttagtacagacagtcgcaaggcagacggtggagcattgtcaatacaggccacctcgaatcagggaagatcgtaaccgaaaaacagccagtcctgaagattcagatatgccagatcatgatttagagcctccaagatttgcccagcttgcattggagcgtgttctacaggactggaatgccttgaaatctatgattatgtttgggtcgcaggagaataaagaccctcttagtgccagcagtagaataggccatcttttgccagaagagcaagtatacctcaatcagcaaagtggcacaattcggctggactgtttcactcattgccttatagttaagtgtacagcagatattttgcttttagatactctactaggtacactagtgaaagaactccaaaacaaatatacacctggacgtagagaagaagctattgctgtgacaatgaggtttctacgttcagtggcaagagtttttgttattctgagtgtggaaatggcttcatccaaaaagaaaaacaactttattccacagccaattggaaaatgcaagcgtgtattccaagcattgctaccttacgctgtggaagaattgtgcaacgtagcagagtcactgattgttcctgtcagaatggggattgctcgtccaactgcaccatttaccctggctagtactagcatagatgccatgcagggcagtgaagaattattttcagtggaaccactaccaccacgaccatcatctgatcagtctagcagctccagtcagtctcagtcatcctacatcatcaggaatccacagcagaggcgcatcagccagtcacagcccgttcggggcagagatgaagaacaggatgatattgtttcagcagatgtggaagaggttgaggtggtggagggtgtggctggagaagaggatcatcatgatgaacaggaagaacacggggaagaaaatgctgaggcagagggacaacatgatgagcatgatgaagacgggagtgatatggagctggacttgttagcagcagctgaaacagaaagtgatagtgaaagtaaccacagcaaccaagataatgctagtgggcgcagaagcgttgtcactgcagcaactgctggttcagaagcaggagcaagcagtgttcctgccttcttttctgaagatgattctcaatcgaatgactcaagtgattctgatagcagtagtagtcagagtgacgacatagaacaggagacctttatgcttgatgagccattagaaagaaccacaaatagctcccatgccaatggtgctgcccaagctccccgttcaatgcagtgggctgtccgcaacacccagcatcagcgagcagccagtacagccccttccagtacatctacaccagcagcaagttcagcgggtttgatttatattgatccttcaaacttacgccggagtggtaccatcagtacaagtgctgcagctgcagcagctgctttggaagctagcaacgccagcagttacctaacatctgcaagcagtttagccagggcttacagcattgtcattagacaaatctcggacttgatgggccttattcctaagtataatcacctagtatactctcagattccagcagctgtgaaattgacttaccaagatgcagtaaacttacagaactatgtagaagaaaagcttattcccacttggaactggatggtcagtattatggattctactgaagctcaattacgttatggttctgcattagcatctgctggtgatcctggacatccaaatcatcctcttcacgcttctcagaattcagcgagaagagagaggatgactgcgcgagaagaagctagcttacgaacacttgaaggcagacgacgtgccaccttgcttagcgcccgtcaaggaatgatgtctgcacgaggagacttcctaaattatgctctgtctctaatgcggtctcataatgatgagcattctgatgttcttccagttttggatgtttgctcattgaagcatgtggcatatgtttttcaagcacttatatactggattaaggcaatgaatcagcagacaacattggatacacctcaactagaacgcaaaaggacgcgagaactcttggaactgggtattgataatgaagattcagaacatgaaaatgatgatgacaccaatcaaagtgctactttgaatgataaggatgatgactctcttcctgcagaaactggccaaaaccatccatttttccgacgttcagactccatgacattccttgggtgtatacccccaaatccatttgaagtgcctctggctgaagccatccccttggctgatcagccacatctgttgcagccaaatgctagaaaggaggatctttttggccgtccaagtcagggtctttattcttcatctgccagtagtgggaaatgtttaatggaggttacagtggatagaaactgcctagaggttcttccaacaaaaatgtcttatgctgccaatctgaaaaatgtaatgaacatgcaaaaccggcaaaaaaaagaaggggaagaacagcccgtgctgccagaagaaactgagagttcaaaaccagggccatctgctcatgatcttgctgcacaattaaaaagtagcttactagcagaaataggacttactgaaagtgaagggccacctctcacatctttcaggccacagtgtagctttatgggaatggttatttcccatgatatgctgctaggacgttggcgcctttctttagaactgttcggcagggtattcatggaagatgttggagcagaacctggatcaatcctaactgaattgggtggttttgaggtaaaagaatcaaaattccgcagagaaatggaaaaactgagaaaccagcagtcaagagatttgtcactagaggttgatcgggatcgagatcttctcattcagcagactatgaggcagcttaacaatcactttggtcgaagatgtgctactacaccaatggctgtacacagagtaaaagtcacatttaaggatgagccaggagagggcagtggtgtagcacgaagtttttatacagccattgcacaagcatttttatcaaatgaaaaattgccaaatctagagtgtatccaaaatgccaacaaaggcacccacacaagtttaatgcagagattaaggaaccgaggagagagagaccgggaaagggagagagaaagggaaatgaggaggagtagtggtttgcgagcaggttctcggagggaccgggatagagactttagaagacagctttccatcgacactaggccctttagaccagcctctgaagggaatcctagcgatgatcctgagcctttgccagcacatcggcaggcacttggagagaggctttatcctcgtgtacaagcaatgcaaccagcatttgcaagtaaaatcactggcatgttgttggaattatccccagctcagctgcttctccttctagcaagtgaggattctctgagagcaagagtggatgaggccatggaactcattattgcacatggacgggaaaatggagctgatagtatcctggatcttggattagtagactcctcagaaaaggtacagcaggaaaaccgaaagcgccatggctctagtcgaagtgtagtagatatggatttagatgatacagatgatggtgatgacaatgcccctttgttttaccaacctgggaaaagaggattttatactccaaggcctggcaagaacacagaagcaaggttgaattgtttcagaaacattggcaggattcttggactatgtctgttacagaatgaactctgtcctatcacattgaatagacatgtaattaaagtattgcttggtagaaaagtcaattggcatgattttgctttttttgatcctgtaatgtatgagagtttgcggcaactaatcctcgcgtctcagagttcagatgctgatgctgttttctcagcaatggatttggcatttgcaattgacctgtgtaaagaagaaggtggaggacaggttgaactcattcctaatggtgtaaatataccagtcactccacagaatgtatatgagtatgtgcggaaatacgcagaacacagaatgttggtagttgcagaacagcccttacatgcaatgaggaaaggtctactagatgtgcttccaaaaaattcattagaagatttaacggcagaagattttaggcttttggtaaatggctgcggtgaagtcaatgtgcaaatgctgatcagttttacctctttcaatgatgaatcaggagaaaatgctgagaagcttctgcagttcaagcgttggttctggtcaatagtagagaagatgagcatgacagaacgacaagatcttgtttacttttggacatcaagcccatcactgccagccagtgaagaaggattccagcctatgccctcaatcacaataagaccaccagatgaccaacatcttcctactgcaaatacttgcatttctcgactttacgtcccactctattcctctaaacagattctcaaacagaaattgttactcgccattaagaccaagaattttggttttgtgtagagtataaaaagtgtgtattgctgtgtaatattactagcaaattttgtagatttttttccatttgtctataaaagtt

SEQ ID NO: 4 - protein, 2793 aa, human Edd cDNA splice variant
MTSIHFVVHPLPGTEDQLNDRLREVSEKLNKYNLNSHPPLNVLEQATIKQCVVGPNHAAFLLEDGRVCRIGFSVQPDRLELGKPDNNDGSKLNSNSGAGRTSRPGRTSDSPWFLSGSETLGRLAGNTLGSRWSSGVGGSGGGSSGRSSAGARDSRRQTRVIRTGRDRGSGLLGSQPQPVIPASVIPEELISQAQVVLQGKSRSVIIRELQRTNLDVNLAVNNLLSRDDEDGDDGDDTASESYLPGEDLMSLLDADIHSAHPSVIIDADAMFSEDISYFGYPSFRRSSLSRLGSSRERDSELLRERESVLRLRERRWLDGASFDNERGSTSKEGEPNLDKKNTPVQSPVSLGEDLQWWPDKDGTKFICIGALYSELLAVSSKGELYQWKWSESEPYRNAQNPSLHHPRATFLGLTNEKIVLLSANSIRATVATENNKVATWVDETLSSVASKLEHTAQTYSELQGERIVSLHCCALYTCAQLENSLYWWGVVPFSQRKKMLEKARAKNKKPKSSAGISSMPNITVGTQVCLRNNPLYHAGAVAFSISAGIPKVGVLMESVWNMNDSCRFQLRSPESLKNMEKASKTTEAKPESKQEPVKTEMGPPPSPASTCSDASSIASSASMPYKRRRSTPAPKEEEKVNEEQWSLREVVFVEDVKNVPVGKVLKVDGAYVAVKFPGTSSNTNCQNSSGPDADPSSLLQDCRLLRIDELQVVKTGGTPKVPDCFQRTPKKLCIPEKTEILAVNVDSKGVHAVLKTGNWVRYCIFDLATGKAEQENNFPTSSIAFLGQNERNVAIFTAGQESPIILRDGNGTIYPMAKDCMGGIRDPDWLDLPPISSLGMGVHSLINLPANSTIKKKAAVIIMAVEKQTLMQHILRCDYEACRQYLMNLEQAVVLEQNLQMLQTFISHRCDGNRNILHACVSVCFPTSNKETKEEEEAERSERNTFAERLSAVEAIANAISVVSSNGPGNRAGSSSSRSLRLREMMRRSLRAAGLGRHEAGASSSDHQDPVSPPIAPPSWVPDPPAMDPDGDIDFILAPAVGSLTTAATGTGQGPSTSTIPGPSTEPSVVESKDRKANAHFILKLLCDSVVLQPYLRELLSAKDARGMTPFMSAVSGRAYPAAITILETAQKIAKAEISSSEKEEDVFMGMVCPSGTNPDDSPLYVLCCNDTCSFTWTGAEHINQDIFECRTCGLLESLCCCTECARVCHKGHDCKLKRTSPTAYCDCWEKCKCKTLIAGQKSARLDLLYRLLTATNLVTLPNSRGEHLLLFLVQTVARQTVEHCQYRPPRIREDRNRKTASPEDSDMPDHDLEPPRFAQLALERVLQDWNALKSMIMFGSQENKDPLSASSRIGHLLPEEQVYLNQQSGTIRLDCFTHCLIVKCTADILLLDTLLGTLVKELQNKYTPGRREEAIAVTMRFLRSVARVFVILSVEMASSKKKNNFIPQPIGKCKRVFQALLPYAVEELCNVAESLIVPVRMGIARPTAPFTLASTSIDAMQGSEELFSVEPLPPRPSSDQSSSSSQSQSSYIIRNPQQRRISQSQPVRGRDEEQDDIVSADVEEVEVVEGVAGEEDHHDEQEEHGEENAEAEGQHDEHDEDGSDMELDLLAAAETESDSESNHSNQDNASGRRSVVTAATAGSEAGASSVPAFFSEDDSQSNDSSDSDSSSSQSDDIEQETFMLDEPLERTTNSSHANGAAQAPRSMQWAVRNTQHQRAASTAPSSTSTPAASSAGLIYIDPSNLRRSGTISTSAAAAAAALEASNASSYLTSASSLARAYSIVIRQISDLMGLIPKYNHLVYSQIPAAVKLTYQDAVNLQNYVEEKLIPTWNWMVSIMDSTEAQLRYGSALASAGDPGHPNHPLHASQNSARRERMTAREEASLRTLEGRRRATLLSARQGMMSARGDFLNYALSLMRSHNDEHSDVLPVLDVCSLKHVAYVFQALIYWIKAMNQQTTLDTPQLERKRTRELLELGIDNEDSEHENDDDTNQSATLNDKDDDSLPAETGQNHPFFRRSDSMTFLGCIPPNPFEVPLAEAIPLADQPHLLQPNARKEDLFGRPSQGLYSSSASSGKCLMEVTVDRNCLEVLPTKMSYAANLKNVMNMQNRQKKEGEEQPVLPEETESSKPGPSAHDLAAQLKSSLLAEIGLTESEGPPLTSFRPQCSFMGMVISHDMLLGRWRLSLELFGRVFMEDVGAEPGSILTELGGFEVKESKFRREMEKLRNQQSRDLSLEVDRDRDLLIQQTMRQLNNHFGRRCATTPMAVHRVKVTFKDEPGEGSGVARSFYTAIAQAFLSNEKLPNLECIQNANKGTHTSLMQRLRNRGERDREREREREMRRSSGLRAGSRRDRDRDFRRQLSIDTRPFRPASEGNPSDDPEPLPAHRQALGERLYPRVQAMQPAFASKITGMLLELSPAQLLLLLASEDSLRARVDEAMELIIAHGRENGADSILDLGLVDSSEKVQQENRKRHGSSRSVVDMDLDDTDDGDDNAPLFYQPGKRGFYTPRPGKNTEARLNCFRNIGRILGLCLLQNELCPITLNRHVIKVLLGRKVNWHDFAFFDPVMYESLRQLILASQSSDADAVFSAMDLAFAIDLCKEEGGGQVELIPNGVNIPVTPQNVYEYVRKYAEHRMLVVAEQPLHAMRKGLLDVLPKNSLEDLTAEDFRLLVNGCGEVNVQMLISFTSFNDESGENAEKLLQFKRWFWSIVEKMSMTERQDLVYFWTSSPSLPASEEGFQPMPSITIRPPDDQHLPTANTCISRLYVPLYSSKQILKQKLLLAIKTKNFGFV

SEQ ID NO: 5 - DNA, 30 bp, CEDD microsatellite primer 1
taccctgcagtaaatctcacatgtactccc

SEQ ID NO: 6 - DNA, 30 bp, CEDD microsatellite primer 2
agaatcgcttgaacctagtaggtgaaggtg

SEQ ID NO: 7 - DNA, 288 bp, CEDD amplified microsatellite sequence
taccctgcagtaaaatctcacatgtactcccaagcctaaaagttttttttttaaaaaaaaaaaaaagaaagaaagaaagaaatgaaaactctgcccctcccccccaaaaaacccttaaggatataggaaagaaagttattttttagatagctacacaatgtgtgtgtgtgtgtgtgtgtgtgtgtgtgtgtaagacagagtctcactctgtcgcccaggctggagtgcagtggcatgatctcagctcactgcaacctccaccttcacctactaggttcaagcgattct

SEQ ID NO: 8 - DNA, 1976 bp, human importin alpha-1 cDNA
gccacacggtctttgagctgagtcgaggtggaccctttgaacgcagtcgccctacagccgctgattccccccgcatcgcctcccgtggaagcccaggcccgcttcgcagctttctccctttgtctcataaccatgtccaccaacgagaatgctaatacaccagctgcccgtcttcacagattcaagaacaagggaaaagacagtacagaaatgaggcgtcgcagaatagaggtcaatgtggagctgaggaaagctaagaaggatgaccagatgctgaagaggagaaatgtaagctcatttcctgatgatgctacttctccgctgcaggaaaaccgcaacaaccagggcactgtaaattggtctgttgatgacattgtcaaaggcataaatagcagcaatgtggaaaatcagctccaagctactcaagctgccaggaaactactttccagagaaaaacagccccccatagacaacataatccgggctggtttgattccgaaatttgtgtccttcttgggcagaactgattgtagtcccattcagtttgaatctgcttgggcactcactaacattgcttctgggacatcagaacaaaccaaggctgtggtagatggaggtgccatcccagcattcatttctctgttggcatctccccatgctcacatcagtgaacaagctgtctgggctctaggaaacattgcaggtgatggctcagtgttccgagacttggttattaagtacggtgcagttgacccactgttggctctccttgcagttcctgatatgtcatctttagcatgtggctacttacgtaatcttacctggacactttctaatctttgccgcaacaagaatcctgcacccccgatagatgctgttgagcagattcttcctaccttagttcggctcctgcatcatgatgatccagaagtgttagcagatacctgctgggctatttcctaccttactgatggtccaaatgaacgaattggcatggtggtgaaaacaggagttgtgccccaacttgtgaagcttctaggagcttctgaattgccaattgtgactcctgccctaagagccatagggaatattgtcactggtacagatgaacagactcaggttgtgattgatgcaggagcactcgccgtctttcccagcctgctcaccaaccccaaaactaacattcagaaggaagctacgtggacaatgtcaaacatcacagccggccgccaggaccagatacagcaagttgtgaatcatggattagtcccattccttgtcagtgttctctctaaggcagattttaagacacaaaaggaagctgtgtgggccgtgaccaactataccagtggtggaacagttgaacagattgtgtaccttgttcactgtggcataatagaaccgttgatgaacctcttaactgcaaaagataccaagattattctggttatcctggatgccatttcaaatatctttcaggctgctgagaaactaggtgaaactgagaaacttagtataatgattgaagaatgtggaggcttagacaaaattgaagctctacaaaaccatgaaaatgagtctgtgtataaggcttcgttaagcttaattgagaagtatttctctgtagaggaagaggaagatcaaaacgttgtaccagaaactacctctgaaggctacactttccaagttcaggatggggctcctgggacctttaacttttagatcatgtagctgagacataaatttgttgtgtactacgtttggtattttgtcttattgtttctctactaagaactctttcttaaatgtggtttgttactgtagcactttttacactgaaactatacttgaacagttccaactgtacatacatactgtatgaagcttgtcctctgactaggtttctaatttctatgtggaatttcctatcttgcagcatcctgtaaataaacattcaagtccacccttaaaaaaaa

SEQ ID NO: 9 - protein, 529 aa, human importin alpha-1 protein [sequence omitted]

SEQ ID NO: 10 - DNA, 1717 bp, human importin alpha-3 cDNA
gcacgagccatggcggacaacgagaaactggacaaccaacggctcaagaatttcaagaacaaaggccgcgacttggagactatgagaagacaacgaaatgaagttgtagttgaattaaggaagaataaaagagatgaacatctcttaaagagaaggaatgtaccacatgaagatatctgtgaagactctgatatagatggtgattatagagtgcaaaatacctctctagaagctattgttcaaaatgcttcaagtgataaccaaggaattcaattaagtgcagttcaagctgctaggaagcttttgtccagtgatcgaaatccaccaattgatgacttaataaaatctggaatattgcccattttagtccattgtcttgaaagagatgacaatccttctttacagtttgaagctgcatgggctttgacaaacattgcatctggaacttctgaacaaactcaagcagtagttcagtccaatgctgtgccacttttcctgaggcttctccattcaccccatcagaatgtctgtgagcaagcagtgtgggcattgggaaatatcataggtgatgggccccagtgtagagattatgtcataagtcttggagttgtgaaacctttactttccttcataagtccatctattcctataacattcttaagaaatgttacttgggttatggtcaacttatgtcgccacaaagacccaccaccaccaatggaaaccattcaggagattcttccagccctttgtgttttaattcatcacacagatgtaaatatactggtagacacagtctgggccctctcttaccttactgatgctggcaatgaacaaatacagatggtaatagactctggaatagttcctcatttggttcctctgctcagccaccaggaagttaaagttcagactgctgcacttagagctgtgggcaacattgttactggaactgatgagcaaacacaagtagttttgaactgtgatgctctttcacacttcccagcactcctgacacatcccaaagagaaaattaataaagaagcagtgtggttcctctccaacatcactgcaggaaatcagcagcaggtacaggcagtaattgatgccaatcttgtaccaatgataatacaccttttggataagggggattttggcactcaaaaagaagctgcttgggccataagtaacttaacaattagtggaaggaaagatcaagtggcttaccttatccaacaaaatgttatcccacctttttgcaacttgctgactgtaaaagatgcacaagttgtgcaagtagtactcgatggactaagtaatatattaaaaatggctgaagatgaggcagaaaccataggcaatcttatagaagaatgtggagggctggagaaaattgaacaacttcaaaatcatgaaaatgaagacatctacaaattggcctatgagatcattgatcagttcttctcttcagatgatattgatgaagaccctagccttgttccagaggcaattcaaggcggaacatttggtttcaattcatctgccaatgtaccaacagaagggttccagttttagaaagatgttgtggaagttaggtacaatgcagcactgagatatatatatatatatgtgtgtgtgtatatatatatatatatacatatatataaaaaggtttgatccatcaaaaaaaaaaaaaaaaaaaaaaaaaaaaaaaaaa

SEQ ID NO: 11 - protein, 521 aa, human importin alpha-3 protein
MADNEKLDNQRLKNFKNKGRDLETMRRQRNEVVVELRKNKRDEHLLKRRNVPHEDICEDSDIDGDYRVQNTSLEAIVQNASSDNQGIQLSAVQAARKLLSSDRNPPIDDLIKSGILPILVHCLERDDNPSLQFEAAWALTNIASGTSEQTQAVVQSNAVPLFLRLLHSPHQNVCEQAVWALGNIIGDGPQCRDYVISLGVVKPLLSFISPSIPITFLRNVTWVMVNLCRHKDPPPPMETIQEILPALCVLIHHTDVNILVDTVWALSYLTDAGNEQIQMVIDSGIVPHLVPLLSHQEVKVQTAALRAVGNIVTGTDEQTQVVLNCDALSHFPALLTHPKEKINKEAVWFLSNITAGNQQQVQAVIDANLVPMIIHLLDKGDFGTQKEAAWAISNLTISGRKDQVAYLIQQNVIPPFCNLLTVKDAQVVQVVLDGLSNILKMAEDEAETIGNLIEECGGLEKIEQLQNHENEDIYKLAYEIIDQFFSSDDIDEDPSLVPEAIQGGTFGFNSSANVPTEGFQF

SEQ ID NO: 12 - DNA, 2940 bp, human importin alpha-5 cDNA
ctaacttcagcggtggcaccgggatcggttgccttgagcctgaaatatgaccaccccaggaaaagagaactttcgcctgaaaagttacaagaacaaatctctgaatcccgatgagatgcgcaggaggagggaggaagaaggactgcagttacgaaagcagaaaagagaagagcagttattcaagcggagaaatgttgctacagcagaagaagaaacagaagaagaagttatgtcagatggaggctttcatgaggctcagattagtaacatggagatggcaccaggtggtgtcatcacttctgacatgattgagatgatattttccaaaagcccagagcaacagctttcagcaacacagaaattcaggaagctgctttcaaaagaacctaaccctcctattgatgaagttatcagcacaccaggagtagtggccaggtttgtggagttcctcaaacgaaaagagaattgttcactgcagtttgaatcagcttgggtactgacaaatattgcttcaggaaattctcttcagacccgaattgtgattcaggcaagagctgtgcccatcttcatagagttgctcagctcagagtttgaagatgtccaggaacaggcagtctgggctcttggcaacattgctggagatagtaccatgtgcagggactatgtcttagactgcaatatccttccccctcttttgcagttattttcaaagcaaaaccgcctgaccatgacccggaatgcagtatgggctttgtctaatctctgtagagggaaaagtccacctccagaatttgcaaaggtttctccatgtctgaatgtgctttcctggttgctgtttgtcagtgacactgatgtactggctgatgcctgctgggccctctcatatctatcagatggacccaatgataaaattcaagcggtcatcgatgcgggagtatgtaggagacttgtggaactgctgatgcataatgattataaagtggtttctcctgctttgcgagctgtgggaaacattgtcacaggggatgatattcagacacaggtaattctgaattgctcagctctgcagagtttattgcatttgctgagtagcccaaaggaatctatcaaaaaggaagcatgttggacgatatctaatattacagctggaaatagggcacagatccagactgtgatagatgccaacattttcccagccctcattagtattttacaaactgctgaatttcggacaagaaaagaagcagcttgggccatcacaaatgcaacttctggaggatcagctgaacagatcaagtacctagtagaactgggttgtatcaagccgctctgtgatctcctcacggtcatggactctaagattgtacaggttgccctaaatggcttggaaaatatcctgaggcttggagaacaggaagccaaaaggaacggcactggcattaacccttactgtgctttgattgaagaagcttatggtctggataaaattgagttcttacagagtcatgaaaaccaggagatctaccaaaaggcctttgatcttattgagcattacttcgggaccgaagatgaagacagcagcattgcaccccaggttgaccttaaccagcagcagtacatcttccaacagtgtgaggctcctatggaaggtttccagctttgaagcaatactctgctttcacgtacctgtgctcagaccaggctacccagtcgagtcctcttgtggagcccacagtcctcatggagctaacttctcaaatgttttccataatactgtttgcgctcatttgcttgccttgcgcacctgctctcttacacacatctggaaaacctccggctctctgtggtgggatacccttctaataaaagggtaaccagaacggcccactctcttttacggaaaaatccctaggctttggagatccgcacttacattagagttatgggaatatacacatattaatgtggctccctttttcttgtgggggaataaaagaggactcctcctcattccctttaacatgggggaaaaaactgacattaaaagatgagactaaatctttatcttgaattttacacaactacttacgacaagggagatgtttagacctgttggtatacttcagagtacttttcatgagttcttccacagtgaacccttggattacctggtggctttttctagccagattgcattaatccttactgagattggatggttttctttcctctattggcgccattcttcagatattaaagttaaaccatccactccctcaccttcagccttcagtgaatgtgctttctagttgtcaggaatgctgaagaattaacactttgactcctaaatgtgatactggtgggtaagagcagggcacatttaatttgttcgcttttgcttctctttggtctgggcacatttaatttgttcgcttttgcttctctttggtcttttcgaatacttagtaatcgaaaaccatatcctgtaatttaataaaaaaaactaaggacgaaaaaacccctccaattttcccaaatgcaatcagtgtaactaggggctgtgtttctgcattaaaataaatgtttcaggctttgtggtcctgatcaaggtcctcattaaaaaattggagttcaccctaggcttttcccctctgtgactggcagataacacatacttttgaaagtaactttgggattttttttcttaggtgcagctcgattctaatcttttcatgctgcacacgattcctttaatcgatagcatccttatctgaaagaaataaccatcttctcaacatgacctgcttaacccaaataagaacagtgatcttataacctcattgtttcctaatctattttatttcatctcctgctagtactgtgccgcttccccctccccccacacaaaataaaaacagtatctcgcttctggctcatttt

SEQ ID NO: 13 - protein, 538 aa, importin alpha-5 protein
MTTPGKENFRLKSYKNKSLNPDEMRRRREEEGLQLRKQKREEQLFKRRNVATAEEETEEEVMSDGGFHEAQISNMEMAPGGVITSDMIEMIFSKSPEQQLSATQKFRKLLSKEPNPPIDEVISTPGVVARFVEFLKRKENCSLQFESAWVLTNIASGNSLQTRIVIQARAVPIFIELLSSEFEDVQEQAVWALGNIAGDSTMCRDYVLDCNILPPLLQLFSKQNRLTMTRNAVWALSNLCRGKSPPPEFAKVSPCLNVLSWLLFVSDTDVLADACWALSYLSDGPNDKIQAVIDAGVCRRLVELLMHNDYKVVSPALRAVGNIVTGDDIQTQVILNCSALQSLLHLLSSPKESIKKEACWTISNITAGNRAQIQTVIDANIFPALISILQTAEFRTRKEAAWAITNATSGGSAEQIKYLVELGCIKPLCDLLTVMDSKIVQVALNGLENILRLGEQEAKRNGTGINPYCALIEEAYGLDKIEFLQSHENQEIYQKAFDLIEHYFGTEDEDSSIAPQVDLNQQQYIFQQCEAPMEGFQL

SEQ ID NO: 14 - DNA, 3014 bp, human progesterone receptor cDNA
ctgaccagcgccgccctcccccgcccccgacccaggaggtggagatccctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtgg

SEQ ID NO: 15 - protein, 933 aa, human progesterone receptor protein
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 16 - DNA, 905 bp, human calcium and integrin binding protein (CIB1) cDNA
tctcccgaattcggcacgaggcggcgtctcgaggcgagttggcggagctgtgcgcgcggcggggcgatggggggctcgggcagtcgcctgtccaaggagctgctggccgagtaccaggacttgacgttcctgacgaagcaggagatcctcctagcccacaggcggttttgtgagctgcttccccaggagcagcggaccgtggagtcgtcacttcgggcacaagtgcccttcgagcagattctcagccttccagagctcaaggccaaccccttcaaggagcgaatctgcagggtcttctccacatccccagccaaagacagccttagctttgaggacttcctggatctcctcagtgtgttcagtgacacagccacgccagacatcaagtcccattatgccttccgcatctttgactttgatgatgacggaaccttgaacagagaagacctgagccggctggtgaactgcctcacgggagagggcgaggacacacggcttagtgcgtctgagatgaagcagctcatcgacaacatcctggaggagtctgacattgacagggatggaaccatcaacctctctgagttccagcacgtcatctcccgttctccagactttgccagctcctttaagattgtcctgtgacagcagccccagcgtgtgtcctggcaccctgtccaagaacctttctactgctgagctgtggccaaggtcaagcctgtgttgccagtgcgggccaagctggcccagcctggagctggcgctgtgcagcctcaccccgggcaggggcggccctcgttgtcagggcctctcctcactgctgttgtcattgctccgtttgtgtttgtactaatcagtaataaaggtttagaagtttgaccctaaaaaaaaaaaaaaaaaaaaaaaaa

SEQ ID NO: 17 - protein, 191 aa, human CIB1 protein
MGGSGSRLSKELLAEYQDLTFLTKQEILLAHRRFCELLPQEQRTVESSLRAQVPFEQILSLPELKANPFKERICRVFSTSPAKDSLSFEDFLDLLSVFSDTATPDIKSHYAFRIFDFDDDGTLNREDLSRLVNCLTGEGEDTRLSASEMKQLIDNILEESDIDRDGTINLSEFQHVISRSPDFASSFKIVL

SEQ ID NO: 18 - DNA, 2547 bp, human Chk2 transcript variant 1 cDNA
cttacaaggtacagtcctctgctcaggggggccaggagggtcttataggcatcattcaccagggtcgaatgcttctctgagaagtccttttcagtctgagacctctggctgaagaaatctgggtggacaagacgctgcagttgctggtacctgtgctggagcttcgctgtatcaactctgaaggaacggttgcagtccataaggctgaagtagtctcgagtggggtcaggtgcctgcagcgctcggcactgtgggcagaagaacctgtcctcccgcccggggccccatgggccgccgcagttccaacagcggggataattgcttcccgcctgcgacgcagcatcgcagcttagcggtctccttctgggaacccctgtcggccaaaacccccacacccggagcaaagccccggctctcccccgccacatctggccggcggcctatctagccgtggtcactcgtggggaaaagcaaagagagcgtctaaccagactaatgttgctgattggctggggagtcgagggggcgggatcacccgaggggaacccgggttctaagttccgctctcccttctaaactacaactcccaggaggcattgaggcggcgcctgacggccacatctgctgctcctcattggtccggcggcaggggagggggttttgattggctgagggtggagtttgtatctgcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctgg

SEQ ID NO: 19 - protein, 543 aa, human Chk2 transcript variant 1 protein
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 20 - DNA, 2460 bp, human Chk2 transcript variant 2 cDNA
cttacaaggtacagtcctctgctcaggggggccaggagggtcttataggcatcattcaccagggtcgaatgcttctctgagaagtccttttcagtctgagacctctggctgaagaaatctgggtggacaagacgctgcagttgctggtacctgtgctggagcttcgctgtatcaactctgaaggaacggttgcagtccataaggctgaagtagtctcgagtggggtcaggtgcctgcagcgctcggcactgtgggcagaagaacctgtcctcccgcccggggccccatgggccgccgcagttccaacagcggggataattgcttcccgcctgcgacgcagcatcgcagcttagcggtctccttctgggaacccctgtcggccaaaacccccacacccggagcaaagccccggctctcccccgccacatctggccggcggcctatctagccgtggtcactcgtggggaaaagcaaagagagcgtctaaccagactaatgttgctgattggctggggagtcgagggggcgggatcacccgaggggaacccgggttctaagttccgctctcccttctaaactacaactcccaggaggcattgaggcggcgcctgacggccacatctgctgctcctcattggtccggcggcaggggagggggttttgattggctgagggtggagtttgtatctgcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctgg

SEQ ID NO: 21 - protein, 514 aa, human Chk2 transcript variant 2 protein
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 22 - DNA, 10987 bp, human BRCA2 cDNA
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 23 - protein, 3418 aa, human BRCA2 protein
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETFYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 24 - DNA, 21 bp, 586F18b microsatellite primer 1
gctagggaaccaaactgccag

SEQ ID NO: 25 - DNA, 26 bp, 586F18b microsatellite primer 2
tgcaaaataacaatagctttgcttag

SEQ ID NO: 26 - DNA, 25 bp, primer EDD 407F
gctagtcaccaacttctgggtctaa

SEQ ID NO: 27 - DNA, 29 bp, primer EDD-490R
cagcaaaaagataaatcacagtgtaaatt

SEQ ID NO: 28 - DNA, 27 bp, primer EDD-433T
  misc_feature (1)..(1): modified by attachment of FAM dye
  misc_feature (27)..(27): modified by attachment of TAMRA dye
cccagccaaagatgacagcagaacaac

SEQ ID NO: 29 - DNA, 24 bp, EDD forward primer
ttaggcttttggtaaatggctgcg

SEQ ID NO: 30 - DNA, 24 bp, EDD reverse primer
tgagggcataggctggaatccttc

SEQ ID NO: 31 - DNA, 22 bp, Carbonic anhydrase II forward primer
ccacccctcctcttctggaatg

SEQ ID NO: 32 - DNA, 25 bp, Carbonic anhydrase II reverse primer
gctttgatttgcctgttcttcagtg

SEQ ID NO: 33 - DNA, 23 bp, p53 ribonucleotide reductase (p53R2) forward primer
gtgactttgcttgcctgatgttc

SEQ ID NO: 34 - DNA, 23 bp, p53 ribonucleotide reductase (p53R2) reverse primer
tctgtggtttctgccataactgc

SEQ ID NO: 35 - DNA, 20 bp, GAPDH forward primer
gacatcaagaaggtggtgaa

SEQ ID NO: 36 - DNA, 20 bp, GAPDH reverse primer
tgtcataccaggaaatgagc

SEQ ID NO: 37 - DNA, 25 bp, EDD forward primer
cattgctgaccctatccctgtgttg

SEQ ID NO: 38 - DNA, 24 bp, EDD reverse primer
tagcccgtgaaatcctcccatctc

SEQ ID NO: 39 - DNA, 24 bp, carbonic anhydrase II forward primer
acccgcctcatgcctcagccttac

SEQ ID NO: 40 - DNA, 24 bp, p53R2 forward primer
tgtcagccttgagtacctccaggg

SEQ ID NO: 41 - DNA, 18 bp, beclin forward primer
taggtttggggtgagtgg

SEQ ID NO: 42 - DNA, 18 bp, reverse primer
agtctgtgggcagcaagg

SEQ ID NO: 43 - protein, 20 aa, synthetic bipartite nuclear localization sequence (NLS) from amino acid residues 1222 to 1241 of EDD
KLKRTSPTAYCDCWEKCKCK

SEQ ID NO: 44 - protein, 16 aa, synthetic bipartite nuclear localization sequence (NLS) from amino acid residues 502 to 517 of EDD
RKKMLEKARAKNKKPK

SEQ ID NO: 45 - protein, 6 aa, synthetic monopartite nuclear localization sequence (NLS) from amino acid residues 630 to 635 of EDD
PYKRRR

SEQ ID NO: 46 - protein, 10 aa, FHA domain-binding Phosphopeptide
  MOD_RES (6)..(6): PHOSPHORYLATION
RWFDTYLIRR

SEQ ID NO: 47 - DNA, 21 bp, synthetic siRNA strand
gcaguguuccugccuucuutt

SEQ ID NO: 48 - DNA, 21 bp, synthetic siRNA strand
ttcgucacaaggacggaagaa

SEQ ID NO: 49 - DNA, 216 bp, MMTV LTR
gtttaaataagtttatggttacaaactgttcttaaaacgaggatgtgagacaagtggtttcctgacttggtttggtatcaaatgttctgatctgagctcttagtgttctattttcctatgttcttttggaatctatccaagtcttatgtaaatgcttatgtaaaccataatataaaagagtgctgattttttgagtaaacttgcaacagtcctaac

SEQ ID NO: 50 - DNA, 1032 bp, Cre recombinase
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 51 - DNA, 22 bp, knockout genotyping primer 1
ctagggaagtgcattaggtaag

SEQ ID NO: 52 - DNA, 20 bp, knockout genotyping primer 2
taagggcaggtgttcctctg

SEQ ID NO: 53 - DNA, 25 bp, knockout genotyping primer 3
gtactgtggtttccaaatgtgtcag